(19) United States
(12) Patent Application Publication
(10) Pub. No.: US 2021/0363582 A1
(43) Pub. Date: Nov. 25, 2021

(54) BIOMARKERS FOR PREDICTING RISK OF ACUTE ISCHEMIC STROKE AND METHODS OF USE THEREOF

(71) Applicant: The Research Foundation for State University of New York, Albany, NY (US)

(72) Inventors: Alison E. Baird, Brooklyn, NY (US); Mateusz Adamski, Kradow (PL); Steven A. Soper, Lawrence, KS (US)

(21) Appl. No.: 17/336,180

(22) Filed: Jun. 1, 2021

Related U.S. Application Data
(63) Continuation-in-part of application No. 15/028,732, filed on Apr. 12, 2016, now Pat. No. 11,021,750.
(60) Provisional application No. 61/890,274, filed on Oct. 13, 2013.

(51) Int. Cl. C12Q 1/6883; A61K 38/48

(52) U.S. Cl. CPC C12Q 1/6883; A61K 38/482; C12Q 2600/118; C12Q 2600/158; C12Y 304/21068

(57) ABSTRACT

Biomarkers useful for the diagnosis and treatment of acute ischemic stroke are disclosed. Also provided is a quantitative assay method for accurately identifying transcript number in a biological sample.

### CROSS-REFERENCE TO RELATED APPLICATIONS

This application is a Continuation-in-Part of U.S. patent application Ser. No. 15/028,732, filed Apr. 12, 2016, now U.S. Pat. No. 11,021,750, which is a § 371 of International Application No. PCT/US2014/06532, filed Oct. 14, 2014, which claims priority to U.S. Provisional application 61/890,274 filed Oct. 13, 2013, the entire contents of each being incorporated herein by reference as though set forth in full.

### GRANT SUPPORT STATEMENT

This invention was made with government support under Grant Numbers ROI EB010087 and P41EB020594 awarded by the National Institute of Health. The government has certain rights in the invention.

### INCORPORATION-BY-REFERENCE OF MATERIAL SUBMITTED IN ELECTRONIC FORM

Incorporated herein by reference in its entirety is the sequence listing submitted via EFS-Web as a text file named SEQLIST.txt, created May 27, 2021, and having a size of 16,487 bytes.

### FIELD OF THE INVENTION

This invention relates to the fields of cardiovascular disease, ischemic stroke and biomarker detection. More specifically, the invention discloses biomarkers that are present in 25 peripheral blood which are indicative of an increased risk for ischemic stroke and methods of use thereof in diagnostic and prognostic assays. Also disclosed are liquid biopsy assays to facilitate stroke diagnosis and screening assays utilizing the biomarkers of the invention to identify agents useful for the treatment and prevention of ischemic stroke.

### BACKGROUND OF THE INVENTION

Several publications and patent documents are cited throughout the specification in order to describe the state of the art to which this invention pertains. Each of these citations is incorporated herein by reference as though set forth in full.

Strokes result from focal reductions of blood flow to the brain and are second to coronary heart disease (CHD) in terms of vascular disease incidence, morbidity, and mortality. Currently, there are about 795,000 new and recurrent strokes per year in the U.S. compared with 1,350,000 new and recurrent coronary events. There is a substantial additional burden of asymptomatic cerebrovascular disease. About 83% of strokes are due to arterial vascular occlusion (ischemic stroke), and about 17% are due to vascular rupture (hemorrhagic stroke).

Many scientific advances have occurred in stroke diagnosis, treatment, and prevention over the past 10 to 20 years, such as advances in neurovascular imaging and intravenous tissue plasminogen activator therapy, but important questions remain unanswered. Modifiable risk factors account for only about 60% of the population-attributable risk (PAR) for stroke (9-10), as opposed to risk factors identified for CHD, which may account for more than 90% of the attributable risk. The mechanisms for over 30% of ischemic strokes are not known, even after extensive workup.

Stroke diagnosis is inaccurate in up to 30% of patients acutely, and it is not possible to reliably distinguish between ischemic and hemorrhagic stroke clinically. No blood-based diagnostic marker has yet been developed for stroke, unlike for acute coronary syndromes; this might be because of issues related to the blood-brain barrier. There is also no reliable way to predict which patients will develop hemorrhagic transformation in the brain after thrombolytic therapy.

Clearly, a need exists in the art for a panel of biomarkers associated with an increased risk of stroke for the management and treatment thereof.

### SUMMARY OF THE INVENTION

In accordance with the present invention, a method for identifying patients having an increased risk for the development of acute ischemic stroke is provided. An exemplary method entails obtaining a biological sample from a test subject and determining the expression levels of at least three gene markers from Table 4, wherein upregulation of said markers relative to predetermined control levels observed in non-afflicted controls, are indicative of an increased risk for the development of acute ischemic stroke. In one embodiment said three gene markers are PLBD1, PYGL, and BST1. The method may further comprise analysis of DUSP1, VCAN, FCGR1A and FOS or DUSP1, FOS, NPL, KIAA0146 and ENTPD1. In yet another embodiment the gene markers consist of PILRA, BCL6, FPR1, LY96, S100A9, S100A12 and MMP9. The method may further comprise determination of expression levels of any of the markers listed in Table 4. In yet another aspect, expression levels of each of BST1, S100A9, PLBD1, S100A12, SUSP1, ETS2, S100P, FOS, CYBB, PYGL, F5, CD93, ENTPD1, CKAP4, ADM and IQGAP1 are determined. In a particularly preferred embodiment of the invention, the markers are PLBD1, PYGL, BST1, DUSP1, VCAN, FCGR1A and FOS.

In certain aspects, the determining step comprises contacting said sample with an agent having affinity for said ischemic stroke associated markers, the agent forming a specific binding pair with said markers and further comprising a detectable label, measuring said detectable label, thereby determining expression level of said marker in said sample. In a particularly preferred embodiment, the expression levels are determined using the input quantity method described in Example II. The method may further comprises creating a report summarizing the data obtained by the determination of said ischemic stroke associated marker expression levels and may include recommendation for a treatment modality of said patient.

In yet another aspect of the invention, kits are provided for practicing the methods disclosed herein.

The invention also provides a method for identifying agents which useful for the treatment of acute ischemic stroke. An exemplary method comprises contacting a cell comprising one or more ischemic stroke associated markers from Table 4 with a test agent and assessing the effect of said agent on modulation of expression levels of said markers relative to untreated cells, agents which modulate expression of any of the markers in step a) having utility for the treatment of acute ischemic stroke.

In another embodiment, the invention provides a “test and treat” method for acute ischemic stroke. The patient is first assessed for the expression levels of the ischemic stroke associated markers described above, and if the marker profile is indicative of the presence or predisposition towards a stroke, the patient is administered treatment and placed on the appropriate therapeutic regimen.

A method for quantitative analysis of standard and high-throughput qPCR expression data based on input sample quantity is also disclosed. In one aspect the method comprises obtaining a biological sample for analysis of target gene expression from a test subject and a control subject and measuring input quantity of said samples and extracting RNA transcripts. The extracted RNA is then reverse transcribed into cDNA and the cDNA amplified via polymerase chain reaction. The amplification efficiency and correlation coefficients for each amplified transcript is then determined and relative fold changes between said test and control are calculated using

$$\frac{{T}_{c}}{{C}_{c}} = \frac{c c C}{c c T} \times {( 1 + E )}^{( nCq , C - nCq , T )}$$

wherein, when cells are used as the biological sample, Tc is the number of transcripts per cell in said test subject, Cc is the number of transcripts per cell in a control subject, ccT is the input cell count for said test subject, ccC is the cell input for said control subject, E is the efficiency of target cDNA amplification, and nCq is the cycle number at which amplification crosses the threshold. The method can also comprise determining the absolute value differences between target gene transcripts in test subject and control subject, wherein efficiency of target cDNA amplification is known and said method further comprises introduction of a standard reference sample containing a known quantity of said transcripts, said absolute value difference being calculated using

$${X}_{c} = \frac{{( 1 + E )}^{( nCq , cDNA - nCq , X )}}{c c X}$$

In yet another aspect, the method can also comprise determining the absolute value differences between target gene transcripts in test subject and control subject, wherein efficiency of target cDNA amplification is unknown and said method further comprises introduction of a standard reference sample containing a known quantity of said transcripts, said absolute value difference being calculated using

$${X}_{c} = \frac{{2}^{( nCq , cDNA - nCq , X )}}{c c X} .$$

The invention also provides a method for identifying patients having an increased risk for acute ischemic stroke, by obtaining a biological sample of extracellular vesicles (EVs) from said patient and determining the expression levels of a cluster of at least three genes from Table 4 and Table 11, wherein upregulation of said markers relative to predetermined control levels observed in non-afflicted controls, are indicative of an increased risk for the development of acute ischemic stroke. In one embodiment at least three gene markers are PLBD1, FOS, and VCAN. In other embodiment, five markers are assessed, e.g., PLBD1, FOS, VCAN, MMP9, and CA 4. The method may further comprise analysis of expression levels of one or more genes listed in Table 2.

In certain aspects, the determining step comprises contacting said sample with an agent having affinity for said ischemic stroke associated markers, the agent forming a specific binding pair with said markers and further comprising a detectable label, measuring said detectable label, thereby determining expression level of said marker in said sample. In another embodiment, the expression levels are determined using an input quality method.

In certain embodiments, the markers comprise nucleic acids or fragments thereof which encode proteins indicative of stroke. In this case, the agent is a complementary nucleic acid which hybridizes to said marker. The marker can then be detected using assays, which include, but are not limited to in situ hybridization assay, hybridization assay, gel electrophoresis, RT-PCR, real time PCR, and microarray analysis.

In certain embodiments, the method further comprises administering an agent useful for the amelioration of stroke symptoms to said patient. In certain embodiments, said agent is recombinant tissue plasminogen activator (rt-PA), Tenecteplase, or mechanical thrombectomy. In certain embodiments, the agent is administered within about 3 hours, about 4 hours, about 4.5 hours, about 6 hours, about 8 hours or about 9 hours after the onset of stroke symptoms. In certain forms of stroke the agent is administered within 24 hours.

In certain embodiments of the invention, detection of the cluster comprises reverse transcribing RNA transcripts extracted from said sample into cDNA and amplifying said cDNA using primer pairs and performance of quantitative polymerase reaction (qPCR) thereby detecting and quantifying nucleic acids encoding said genes in said sample.

In certain embodiments, the method further comprises isolating said EVs from a biological sample from said patient. In certain embodiments, the EVs are isolated using EV microfluidic affinity purification (EV-MAP). In certain embodiments, the method is completed within about 4.5 hours, 3.7 hours, about 2.75 hours or about 1 hour.

### BRIEF DESCRIPTION OF THE DRAWING

FIG. 1 shows stroke related transcripts fold changes with p<0.05. This bar graph shows the fold change levels for 16 up-regulated transcripts in ischemic stroke.

FIG. 2A Heatmap and Hierarchical Cluster Analysis This heatmap and hierarchical cluster analysis illustrates gene expression levels for the 41 studied genes in the control subjects (C) and stroke patients (S). Seven clusters (cl.1 to cl.7) are highlighted by seven squares of different color. Data are log-transformed. This demonstrates elevated expression of many transcripts in stroke patients relative to controls. FIG. 2B is a heat map showing that the majority of the biomarkers identified where upregulated in stroke patients when compared to control subjects.

FIG. 3 Characteristics of a 7 transcript classifier for ischemic stroke detection (FIG. 3A) Boxplots demonstrating the threshold values for defining elevated expression of each of the transcripts (PLBD1, PYGL, FOS, DUSP1, BST1, VCAN, FCGR1A). The threshold was set at above the third quartile value in the control group (dashed line on each boxplot). The threshold value was the normalized transcript copy number. (FIG. 3B) Bar graphs depicting the number of transcripts elevated in the stroke patients and the control subjects. In the stroke bar the value for the 7 transcript elevation represents the 5 stroke patients who had all 7 transcripts elevated, the value for 6 transcripts represents the 5 patients who had 6 transcripts elevated, the value for 5 transcripts elevated represents the one stroke patient who had 5 transcripts elevated, etc. In Cluster 1, 83% (15/18) of the stroke patients had 3 or more of the 7 transcripts elevated while 20% (3/15) of the control group showed elevation of 3 or more of the 7 transcripts. Hence the sensitivity was 83% and the specificity was 80%. (FIG. 3C) ROC Analysis for Cluster 1 for Stroke Classification revealed that the AUC was 0.854. Elevation of 3 or more transcripts gave the greatest sensitivity and specificity.

FIG. 4. Expression of FUT4, CD3E, FDFT1 and B2M in a standard dilution series of reference cDNA sample normalized to the volume of diluent using sample input quantity method.

FIG. 5A-5G. Microfluidic devices design and EV recovery. FIG. 5A Picture of CAD showing the 3-bed EV-MAP with circular micropillars. FIG. 5B Hot embossed device fabricated in COC thermoplastic. FIG. 5C Circular micropillars of the device surface. FIG. 5D Picture of 7-bed EV-MAP showing the distribution channels and the diamond-shaped micropillars of the device surface. FIG. 5E Network of 10-μm microchannels between micropillars to enable efficient EV recovery by reducing the distances required for EVs to diffuse and interact with the surface-confined mAbs coated on the micropillars of the 7-bed device. The EV transport dynamics were simulated via a custom Monte Carlo model that incorporated diffusive and convective EV transfer and mAb-EV binding dynamics. Shown are tracks of individual EVs (not to scale) diffusing through a microchannel, where color scales with the EV velocity (blue-low, red-high) and “X” indicating a successful EV-mAb binding event whereas “O” indicates the EV was not captured. Results are averaged until the predicted EV recovery converges. FIG. 5F Monte Carlo simulation results for the 3-bed EV-MAP. FIG. 5G Calculated sample processing time for 3-bed (5 μL/min) and 7-bed devices (20 μL/min). FIG. 5H Results of Monte Carlo simulation for the 7-bed EV-MAP.

FIG. 6A-6K. Affinity enrichment of EVs and release from the microfluidic device. FIG. 6A Schematic diagram representing the workflow for sample processing and release of enriched EVs from the EV-MAP device's surface. FIG. 6B Fluorescence images after staining the EVs captured on the EV-MAP device's surface with an APC-labeled secondary antibody, Left-negative control without anti CD8α mAb; FIG. 6C isotype (IgG2B) control; FIG. 6D florescence images of CD8+ EVs captured from cell media. TEM images of: FIG. 6E USER@ enzyme and buffer used for EV release from the EV-MAP device's surface with no EV infusion; FIG. 6F and FIG. 6G EVs captured and released from the MOLT-3 cell culture media. FIG. 6H NTA results (n=3) and TEM images showing the number of EVs released during first (FIG. 6I) and second (FIG. 6J) USER@ enzyme release. FIG. 6K Percentage of EVs released during first and second release with USER@ enzyme.

FIG. 7A-7C. Cells released from the sinusoidal cell isolation microfluidic device after staining with (FIG. 7A) DAPI, (FIG. 7B) anti-human CD45-FITC antibody, and (FIG. 7C) anti-human CD8α APC mAb.

FIG. 8A-8F. EV mRNA abundance analysis in cell line models. FIG. 8A Cell line viability when cultured with different LPS concentrations in culture medium. FIG. 8B Workflow for isolation of MOLT-3 cells and EVs from culture and gene expression analysis. mRNA gene expression profiles for CD8+ EVs cells (0.5 and 0.7 ng of RNA for stimulated and unstimulated, respectively, used in RT reactions) (FIG. 8C) and CD8+ MOLT-3 cells (0.7 ng RNA for stimulated and unstimulated was used in RT reactions) (FIG. 8D). cDNA was diluted 5× in water before use in ddPCR. Yellow-non-stimulated conditions; red-stimulated conditions. FIG. 8E Correlation between mRNA copies found in stimulated and unstimulated MOLT-3 cells and EVs. FIG. 8F Correlation between mRNA copies found in EVs and MOLT-3 cells in stimulated and unstimulated conditions. (*) indicates P values <0.05.

FIG. 9A-9I Affinity enrichment and gene expression of CD8(+) T-cells and CD8(+) EVs isolated from healthy donors. FIG. 9A Micrograph of cells isolated using a curvilinear cell isolation device (bright field). FIG. 9B TEM image of EVs isolated using the EV-MAP 3-bed device. FIG. 9C Electropherogram for the separation of RNA isolated from CD8+ T-cells and EVs and PEG precipitated EVs from healthy donor plasma. FIG. 9D Correlation plot of particle concentration (presumably EVs) with RNA mass isolated from affinity-selected CD8+ EVs. Boxplots comparing the gene expression of CD8+ T-cells and CD8+ EVs isolated from healthy donor plasma for FIG. 9E PLBD1, FIG. 9F vFOS, FIG. 9G MMP9, FIG. 9H CA4, and FIG. 9I VCAN, for cells (n=5) and EVs (n=6); 0.7 ng and 0.8 ng of RNA isolated from cells and EV, respectively, was used in RT (+/−) reactions.

FIG. 10A-10J. Affinity enrichment and mRNA transcripts analysis in CD8(+) EVs isolated from clinical samples. FIG. 10A NTA and FIG. 10B, 10C TEM images of EVs isolated from clinical sample #4 by PEG precipitation and affinity selected with anti-CD8α mAb using the 7-bed EV-MAP. FIG. 10D Gel electrophoresis of total RNA (TRNA) size distributions from EVs isolated via EV-MAP and PEG precipitation for sample #4. FIG. 10E Heat maps presenting the EV mRNA expression profiles for sample #4. FIG. 10F NTA results for selected samples 1, 4, 6, and 8. FIG. 10G mRNA expression profiling for selected genes in clinical samples. FIG. 10H Heat map analysis of clinical samples (marked with numbers) and healthy donors (identified with letters). FIG. 10I Principal component analysis for clinical samples (identified with numbers) and healthy donors (identified with letters). FIG. 10J Process flow chart showing the steps and time required for our EV mRNA expression profiling assay that uses the EV-MAP for EV enrichment and subsequent ddPCR quantification of five genes used for AIS diagnostics. The EV-MAP microfluidic for EV enrichment used the 7-bed device and accepted plasma samples with no pre-processing required (see FIG. 5D). Following enrichment, the EVs were released from the capture surface enzymatically (see FIG. 6), or directly lysed on the enrichment bed followed by solid-phase extraction (SPE) of the resulting total RNA (TRNA). mRNA was reverse transcribed and then subjected to ddPCR. Amounts of TRNA used in RT(+/−) are shown in Table 15. The total processing time of this assay is 220 min (3.7 h), including the time for sampling and pipetting.

### DETAILED DESCRIPTION OF THE INVENTION

Stroke is a leading cause of death and disability in the community and new diagnostics and therapeutics are greatly needed. Inflammation and immune response after stroke impacts significantly on tissue and clinical outcome[2,3]. Application of molecular and cellular approaches to study the immune system in stroke may offer new diagnostic and therapeutic approaches. Using microarrays that contained between 22,000 and 54,000 oligonucleotide probes, genomic profiling has been applied to the circulating leukocytes of human stroke patients[4-7]. Peripheral blood mononuclear cells (PBMCs) and whole blood samples[5,6] were used for these studies. In three independent analyses 22, 18 and 9 transcripts showed utility for stroke detection[4-6]. In these studies ribonucleic acid (RNA) was sampled between 3 and 72 hours after stroke onset. Different microarrays from two companies (Affymetrix and Illumina) were used and therefore signal intensity was assessed differently for each study. Despite these methodological and experimental differences there was some overlap among the transcripts identified and panels were able to be applied between the study cohorts[4-7].

These microarray studies raised the possibility of added diagnostic utility in stroke from genomic profiling of circulating leukocytes to clinical and neuroimaging information during the time window for thrombolytic therapy[8-11]. Expression changes were seen as early as 3 hours post stroke and persisted at 5 and 24 hours[5]. However, further translation and application of these microarray results has been hindered by data normalization issues, cost, high turnaround time and the limited availability of arrays. While providing unprecedented coverage of the transcriptome, microarray data are also limited by low sensitivity and low accuracy for transcripts expressed at low levels[12,13].

The majority of these stroke-related transcripts were not validated with standard quantitative polymerase chain reactions (qPCR)—the gold standard for measuring gene expression. qPCR-based approaches are more likely than microarrays to be applied and developed for rapid assays and automated point of care systems that would be needed for early stroke diagnosis[14,15]. Compared to microarrays qPCR approaches are characterized by shorter assay turnaround times and high sensitivity, with a theoretical limit of detection of a single copy of messenger ribonucleic acid (mRNA) target[16]. Until now standard reverse transcription (RT)-qPCR has been feasible for studying 6 genes at most from typical clinical samples.

Recently next generation microfluidic high throughput qPCR approaches have become available. These methods, known as high throughput RT-qPCR (HT RT-qPCR) or nanofluidic qPCR, permit the rapid quantification of multiple transcripts using small sample volumes[17,18] with very high sensitivity. Plates can contain up to 96 samples in which 96 transcripts can be simultaneously studied in 9,216 reactions. We have applied HT RT-qPCR to forty candidate markers identified in the three prior gene expression profiling studies to (1) quantitate individual transcript expression, (2) identify transcript clusters and (3) assess the clinical diagnostic utility of the clusters identified for ischemic stroke detection.

Using cluster analysis, we have discovered that groups of genes, ranging from 3 to 8 per cluster, containing individual transcripts from the 3 different panels give highly significant results in this small sample, with p values of the order of 10⁻¹¹ (see Table 4 below). The genes cluster similarly, whether or not the subjects are clustered by stroke and control status. One aspect of the invention entails multiplexing the 3-8 transcripts for use in spFRET approaches.

Rapid diagnosis of acute ischemic stroke (AIS) is crucial for effective thrombolytic treatment. Clot-busting thrombolytic treatment using recombinant tissue plasminogen activator (rt-PA) is the cornerstone of AIS therapy, but the timeframe for treatment is 4.5 hour after the onset of stroke symptoms. However, as rt-PA is absolutely contraindicated for hemorrhagic stroke and current tests for AIS cannot in most cases meet the 4.5 hour time constraint, treatment reaches only ˜5% of AIS patients.

An endovascular procedure called mechanical thrombectomy, is another option to remove a clot in eligible patients with a large vessel occlusion. The procedure involves the threading of a catheter through a blocked artery in the brain. The stent will open and grab the clot, which will allow removal of the stent with the trapped clot. This method can restore vascular patency (i.e., the degree to which blood vessels are not obstructed) of the vessels with a success rate between 41% and 54%. According to guidelines, mechanical thrombectomy should be performed within 6 h of AIS symptoms and can be done only after the patient receives rt-PA.

The current standard-of-care for stroke patients is to undergo computed tomography (CT) to rule out hemorrhagic stroke. CT clearly shows hyper-dense lesions due to hemorrhagic stroke but is much less sensitive for AIS and other medical conditions, such as stroke mimics and prior infarctions. As such, CT has only 26% clinical sensitivity for AIS. Magnetic resonance imaging (MRI) is more sensitive (83%) for both AIS and hemorrhagic stroke, but is more time consuming than CT and is not widely used in an emergency setting. In addition, many hospitals do not have the required instrumentation and trained personnel for MRI to provide real-time scanning and interpretation.

Using extracellular vesicles (EVs) as a source of mRNA for AIS testing reduces the time required for diagnosis and allows for effective treatment of AIS. We now report a microfluidic device for the rapid and efficient affinity-enrichment of CD8(+) EVs and subsequent EV's mRNA analysis using droplet digital PCR (ddPCR). Analysis of mRNA from CD8(+) EVs and their parental T-cells revealed correlation in the expression for AIS-specific genes in both cell lines and healthy donors. Using the techniques provided herein, 80% test positivity for AIS patients and controls was revealed with a total analysis time of 3.7 h.

Certain leukocyte subpopulations respond to AIS via dysregulation of genes. CD8(+) T-cells were, for example, shown to contribute to inflammatory responses with AIS-associated mRNA expression observed <3 h following stroke onset. Importantly, because mRNA transcription precedes protein translation, mRNA expression changes can be observed 6-12 h earlier than detection of proteins in the peripheral blood that may be responding to AIS. Hence, assays based on CD8(+) leukocyte mRNA expression take advantage of the reduced latency of these AIS-specific markers appearing in peripheral blood.

Recently, extracellular vesicles (EVs) have garnered attention as biomarkers for disease diagnostics. EVs isolated from peripheral blood provide information on changes in the brain, including mRNA expression changes responding to AIS. Recent studies have shown that the miRNA-17 family members sourced from EVs were highly expressed in ischemic stroke patients compared to those with stroke mimics, but the cause of higher expression was assigned to a chronic sequela of cerebrovascular small vessel disease rather than ischemic stroke.

### Definitions

A “biomarker” is any gene or protein whose level of expression in a tissue or cell is altered compared to that of a normal or healthy cell or tissue. Biomarkers of the invention are selective for underlying risk of progression to ischemic stroke. By “selectively overexpressed in peripheral blood” is intended that the biomarker of interest is overexpressed in peripheral blood in stroke patients relative to levels observed in control patients. Thus, detection of the biomarkers of the invention permits the differentiation of samples indicative of increased risk of ischemic stroke. Biomarker profiles for this purpose are also within the scope of the invention.

The biomarkers of the invention include genes and proteins, and variants and fragments thereof. Such biomarkers include DNA comprising the entire or partial sequence of the nucleic acid sequence encoding the biomarker, or the complement of such a sequence. The biomarker nucleic acids also include RNA comprising the entire or partial sequence of any of the nucleic acid sequences of interest. A biomarker protein is a protein encoded by or corresponding to a DNA biomarker of the invention. A biomarker protein comprises the entire or partial amino acid sequence of any of the biomarker proteins or polypeptides.

As used herein, the term “risk” refers to an aspect of personal behavior, or lifestyle, an environmental exposure, or an inborn or inherited characteristic which on the basis of epidemiological evidence is known to be associated with health related condition(s) considered important to ameliorate or prevent.

The phrase “genetic signature” refers to a plurality of nucleic acid molecules whose expression levels are indicative of a given metabolic or pathological state. The genetic signatures described herein can be employed to characterize at the molecular level the biomarker profile that is associated with an increased risk of ischemic stroke, thus providing a useful molecular tool for predicting outcomes, for identifying patients at risk, and for use in biomarker in assays for evaluating ischemic stroke preventive agents.

For purposes of the present invention, “a” or “an” entity refers to one or more of that entity; for example, “a cDNA” refers to one or more cDNA or at least one cDNA. The terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an isolated, or biologically pure molecule is a compound that has been removed from its natural milieu. As such, “isolated” and Abiologically pure@ do not necessarily reflect the extent to which the compound has been purified. An isolated compound of the present invention can be obtained from its natural source, can be produced using laboratory synthetic techniques or can be produced by any such chemical synthetic route.

The term “genetic alteration” as used herein refers to a change from the wild-type or reference sequence of one or more nucleic acid molecules. Genetic alterations include without limitation, base pair substitutions, additions and deletions of at least one nucleotide from a nucleic acid molecule of known sequence.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

“Sample” or “patient sample” or “biological sample” generally refers to a sample which may be tested for a particular molecule, preferably a genetic signature specific marker molecule, such as a marker shown in the tables provided below. Samples may include but are not limited to peripheral blood cells, CNS fluids, serum, plasma, buccal swabs, urine, saliva, tears, pleural fluid and the like.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the functional and novel characteristics of the sequence.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁻⁶-fold purification of the native message. Thus, purification of at least one order of magnitude, preferably two or three orders, and more preferably four or five orders of magnitude is expressly contemplated. Thus, the term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The phrase “input sample quantity” refers to accurate measurement of the amount of the starting material used for extraction of RNA. For example, for cell suspensions, cell counts are performed; for solid tissues, tissue volume is determined.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to any specific marker gene or nucleic acid, but does not hybridize to other human nucleotides. Also polynucleotide which “specifically hybridizes” may hybridize only to a specific marker, such a genetic signature-specific marker shown in the Tables below. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

Tₘ=81.5″C+16.6 Log[Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57″C. The Tₘ of a DNA duplex decreases by 1-1.5″C with every 1% decrease in homology. Thus, targets with greater than about 75% sequence identity would be observed using a hybridization temperature of 42″C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “oligonucleotide” or “oligo” as used herein means a short sequence of DNA or DNA derivatives typically 8 to 35 nucleotides in length, primers, or probes. An oligonucleotide can be derived synthetically, by cloning or by amplification. An oligo is defined as a nucleic acid molecule comprised of two or more ribo- or deoxyribonucleotides, preferably more than three. The exact size of the oligonucleotide will depend on various factors and on the particular application and use of the oligonucleotide. The term “derivative” is intended to include any of the above described variants when comprising an additional chemical moiety not normally a part of these molecules. These chemical moieties can have varying purposes including, improving solubility, absorption, biological half life, decreasing toxicity and eliminating or decreasing undesirable side effects.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

A “siRNA” refers to a molecule involved in the RNA interference process for a sequence-specific post-transcriptional gene silencing or gene knockdown by providing small interfering RNAs (siRNAs) that has homology with the sequence of the targeted gene. Small interfering RNAs (siRNAs) can be synthesized in vitro or generated by ribonuclease III cleavage from longer dsRNA and are the mediators of sequence-specific mRNA degradation. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Applied Biosystems (Foster City, Calif., USA), Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Specific siRNA constructs for inhibiting elevated mRNA levels associated with Ischemic Stroke may be between 15-35 nucleotides in length, and more typically about 21 nucleotides in length.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and Atransduction@ refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, peptide-tethering, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the Ischemic Stroke specific marker nucleic acid molecule(s) such that the latter is transcribed into mRNA. Host cell machinery then translates mRNA into a polypeptide.

Those skilled in the art will recognize that a nucleic acid vector can contain nucleic acid elements other than the promoter element and the Ischemic Stroke specific marker gene nucleic acid molecule(s). These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The term “isolated protein” or “isolated and purified protein” is sometimes used herein. This term refers primarily to a protein produced by expression of an isolated genetic signature nucleic acid or biomarker molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long. One or both members of the pair may optionally comprise a detectable label.

“Sample” or “patient sample” or “biological sample” generally refers to a sample which may be tested for a particular molecule or combination of molecules, preferably a combination of the biomarker or genetic signature marker molecules, such as a combination of the markers shown in the Tables below. Samples may include but are not limited to peripheral blood, cells, and other body fluids, serum, plasma, CNS fluid, urine, saliva, tears, buccal swabs and the like.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, small molecules, antibodies, peptides, peptide/DNA complexes, and any nucleic acid based molecule, for example an oligo, which exhibits the capacity to modulate the activity of the genetic signature nucleic acids described herein or their encoded proteins. Agents are evaluated for potential biological activity by inclusion in screening assays described herein below.

The term “modulate” as used herein refers increasing or decreasing. For example, the term modulate refers to the ability of a compound or test agent to either interfere with, or augment signaling or activity of a gene or protein of the present invention.

The phrase “Extracellular vesicles” or “EVs” refer to a collection of cell-derived membranous structures comprising exosomes, ectosomes, microvesicles, etc., all of which have different biogenesis and thus can contain differences in their molecular cargo. The molecular cargo of EVs (mRNA, miRNA, proteins, etc.) can represent the molecular composition of cells from which they originate. In the central nervous system (CNS), EVs maintain normal neuronal function but are also involved in neurodegenerative diseases. For example, EVs released from CD8(+) T-cells play key roles in CNS homeostasis, stroke pathology, and subsequent stroke recovery.

The phrase “recombinant tissue plasminogen activator” or “rt-PA” refers to a protein used in clot-busting thrombolytic treatment. rt-PA treatment is the cornerstone of AIS therapy, but the timeframe for treatment is 4.5 hour after the onset of stroke symptoms. In certain embodiments, the timeframe for treatment is about 9 hours.

The term “Tenecteplase” or “TNKASE” refers to a single-bolus thrombolytic, or clot-busting agent, approved by the U.S. Food and Drug Administration for use in mortality reduction associated with acute myocardial infarction (AMI). Tenecteplase has promising efficacy in treating stroke if administered within about 4.5-9 hours after onset of symptoms.

### Methods of Using the Biomarkers and Genetic Signatures of the Invention

Genetic signature or biomarker encoding nucleic acids, including but not limited to those listed in Tables 4 and 5 herein below may be used for a variety of purposes in accordance with the present invention. The genetic signature associated with an increased risk of ischemic stroke (e.g., the plurality of nucleic acids contained therein) containing DNA, RNA, or fragments thereof may be used as probes to detect the presence of and/or expression of these specific markers in a biological sample. Methods in which such marker nucleic acids may be utilized as probes for such assays include, but are not limited to: (1) in situ hybridization; (2) Southern hybridization (3) northern hybridization; and (4) assorted amplification reactions such as high throughput reverse transcription, quantitative polymerase chain reactions (HT RT qPCR) or conventional PCR.

Further, assays for detecting the genetic signature may be conducted on any type of biological sample, but is most preferably performed on peripheral blood. From the foregoing discussion, it can be seen that genetic signature containing nucleic acids, vectors expressing the same, genetic signature encoded proteins and anti-genetic signature encoded protein specific antibodies of the invention can be used to detect the signature in body tissue, cells, or fluid, and alter genetic signature containing marker protein expression for purposes of assessing the genetic and protein interactions involved in ischemic stroke.

In certain embodiments for screening for genetic signature containing nucleic acid(s), the sample will initially be amplified, e.g. using high throughput RT-qPCR, to increase the amount of the template as compared to other sequences present in the sample. This allows the target sequences to be detected with a high degree of sensitivity if they are present in the sample. This initial step may be avoided by using highly sensitive array techniques that are becoming increasingly important in the art.

Alternatively, additional detection technologies can be employed which detect the ischemic stroke biomarker proteins directly. Such methods include geLC/MS/MS proteomics analysis. This approach provides a full panel of the protein biomarkers present in the sample and allows the clinician to predict outcomes based on the panel of biomarkers present in a sample.

Thus, any of the aforementioned techniques may be used to detect or quantify genetic signature expression and or protein expression levels and accordingly, diagnose patient susceptibility for developing ischemic stroke.

### Kits and Articles of Manufacture

Any of the aforementioned products can be incorporated into a kit which may contain genetic signature polynucleotides or one or more such markers immobilized on a Gene Chip, an oligonucleotide, a polypeptide, a peptide, an antibody, a label, marker, or reporter, a pharmaceutically acceptable carrier, a physiologically acceptable carrier, instructions for use, a container, a vessel for administration, an assay substrate, reagents and vessels suitable for obtaining a peripheral blood sample, reagents suitable for HT RT-qPCR, conventional PCR or any combination thereof.

### Methods of Using the Genetic Signature or Biomarker Proteins for Development of Therapeutic Agents

Since the genetic signature identified herein and the proteins encoded thereby have been associated with the etiology of ischemic stroke, methods for identifying agents that modulate the activity of the genes and their encoded products should result in the generation of efficacious therapeutic agents for the treatment of neurological and cardiovascular disorders, particularly those associated with ischemic stroke.

The nucleic acids comprising the signature contain regions which provide suitable targets for the rational design of therapeutic agents which modulate their activity. Small peptide molecules corresponding to these regions may be used to advantage in the design of therapeutic agents which effectively modulate the activity of the encoded proteins. Molecular modeling should facilitate the identification of specific organic molecules with capacity to bind to the active site of the proteins encoded by the genetic signature nucleic acids based on conformation or key amino acid residues required for function. A combinatorial chemistry approach will be used to identify molecules with greatest activity and then iterations of these molecules will be developed for further cycles of screening. In certain embodiments, candidate agents can be screening from large libraries of synthetic or natural compounds. Such compound libraries are commercially available from a number of companies including but not limited to Maybridge Chemical Co., (Trevillet, Cornwall, UK), Comgenex (Princeton, N.J.), Microsour (New Milford, Conn.) Aldrich (Milwaukee, Wis.) Akos Consulting and Solutions GmbH (Basel, Switzerland), Ambinter (Paris, France), Asinex (Moscow, Russia) Aurora (Graz, Austria), BioFocus DPI (Switzerland), Bionet (Camelford, UK), Chembridge (San Diego, Calif.), Chem Div (San Diego, Calif.). The skilled person is aware of other sources and can readily purchase the same. Once therapeutically efficacious compounds are identified in the screening assays described herein, they can be formulated in to pharmaceutical compositions and utilized for the treatment of ischemic stroke patients.

The polypeptides or fragments employed in drug screening assays may either be free in solution, affixed to a solid support or within a cell. One method of drug screening utilizes eukaryotic or prokaryotic host cells which are stably transformed with recombinant polynucleotides expressing the biomarker polypeptide or fragment, preferably in competitive binding assays. Such cells, either in viable or fixed form, can be used for standard binding assays. One may determine, for example, formation of complexes between the polypeptide or fragment and the agent being tested, or examine the degree to which the formation of a complex between the polypeptide or fragment and a known substrate is interfered with by the agent being tested.

Another technique for drug screening provides high throughput screening for compounds having suitable binding affinity for the encoded polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with the target polypeptide and washed. Bound polypeptide is then detected by methods well known in the art.

A further technique for drug screening involves the use of host eukaryotic cell lines or cells (such as described above) which have a nonfunctional or altered ischemic stroke associated gene. These host cell lines or cells are defective at the polypeptide level. The host cell lines or cells are grown in the presence of drug compound. The effect on cell morphology and/or proliferation of the host cells is measured to determine if the compound is capable of regulating the same in the defective cells. Host cells contemplated for use in the present invention include but are not limited to bacterial cells, fungal cells, insect cells, mammalian cells, particularly neuronal, vascular, neutrophils, fibroblast, and CNS cells. The genetic signature encoding DNA molecules may be introduced singly into such host cells or in combination to assess the phenotype of cells conferred by such expression. Methods for introducing DNA molecules are also well known to those of ordinary skill in the art. Such methods are set forth in Ausubel et al. eds., Current Protocols in Molecular Biology, John Wiley & Sons, NY, N.Y. 1995, the disclosure of which is incorporated by reference herein.

Cells and cell lines suitable for studying the effects of genetic signature expression on cellular morphology and signaling methods of use thereof for drug discovery are provided. Such cells and cell lines will be transfected with one, two, three or all of the genetic signature encoding nucleic acids described herein and the effects on cell functions and cell signaling can be determined. Such cells and cell lines can also be contacted with the siRNA molecules provided herein to assess the effects thereof on similar functions. The siRNA molecules will be tested alone and in combination of 2, 3, 4, and 5 siRNAs to identify the most efficacious combination for down regulating target nucleic acids.

A wide variety of expression vectors are available that can be modified to express the novel DNA or RNA sequences of this invention. The specific vectors exemplified herein are merely illustrative, and are not intended to limit the scope of the invention. Expression methods are described by Sambrook et al. Molecular Cloning: A Laboratory Manual or Current Protocols in Molecular Biology 16.3-17.44 (1989). Expression methods in Saccharomyces are also described in Current Protocols in Molecular Biology (1989).

Suitable vectors for use in practicing the invention include prokaryotic vectors such as the pNH vectors (Stratagene Inc., 11099 N. Torrey Pines Rd., La Jolla, Calif. 92037), pET vectors (Novogen Inc., 565 Science Dr., Madison, Wis. 53711) and the pGEX vectors (Pharmacia LKB Biotechnology Inc., Piscataway, N.J. 08854). Examples of eukaryotic vectors useful in practicing the present invention include the vectors pRc/CMV, pRc/RSV, and pREP (Invitrogen, 11588 Sorrento Valley Rd., San Diego, Calif. 92121); pcDNA3.1N5&His (Invitrogen); baculovirus vectors such as pVL1392, pVL1393, or pAC360 (Invitrogen); and yeast vectors such as YRP17, YIP5, and YEP24 (New England Biolabs, Beverly, Mass.), as well as pRS403 and pRS413 Stratagene Inc.); Picchia vectors such as pHIL-D1 (Phillips Petroleum Co., Bartlesville, Okla. 74004); retroviral vectors such as PLNCX and pLPCX (Clontech); and adenoviral and adeno-associated viral vectors.

Promoters for use in expression vectors of this invention include promoters that are operable in prokaryotic or eukaryotic cells. Promoters that are operable in prokaryotic cells include lactose (lac) control elements, bacteriophage lambda (pL) control elements, arabinose control elements, tryptophan (trp) control elements, bacteriophage T7 control elements, and hybrids thereof. Promoters that are operable in eukaryotic cells include Epstein Barr virus promoters, adenovirus promoters, SV40 promoters, Rous Sarcoma Virus promoters, cytomegalovirus (CMV) promoters, baculovirus promoters such as AcMNPV polyhedrin promoter, Picchia promoters such as the alcohol oxidase promoter, and Saccharomyces promoters such as the gal4 inducible promoter and the PGK constitutive promoter, as well as neuronal-specific platelet-derived growth factor promoter (PDGF).

In addition, a vector of this invention may contain any one of a number of various markers facilitating the selection of a transformed host cell. Such markers include genes associated with temperature sensitivity, drug resistance, or enzymes associated with phenotypic characteristics of the host organisms.

Host cells expressing the genetic signature of the present invention or functional fragments thereof provide a system in which to screen potential compounds or agents for the ability to modulate the development of acute ischemic stroke

Another approach entails the use of phage display libraries engineered to express fragment of the polypeptides encoded by the genetic signature containing nucleic acids on the phage surface. Such libraries are then contacted with a combinatorial chemical library under conditions wherein binding affinity between the expressed peptide and the components of the chemical library may be detected. U.S. Pat. Nos. 6,057,098 and 5,965,456 provide methods and apparatus for performing such assays.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, discussed above, the three-dimensional structure of a protein of interest or, for example, of the protein-substrate complex, is solved by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacophore upon which subsequent drug design can be based.

One can bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacophore.

Thus, one may design drugs which have, e.g., improved polypeptide activity or stability or which act as inhibitors, agonists, antagonists, etc. of polypeptide activity. By virtue of the availability of the genetic signature containing nucleic acid sequences described herein, sufficient amounts of the encoded polypeptide may be made available to perform such analytical studies as x-ray crystallography. In addition, the knowledge of the protein sequence provided herein will guide those employing computer modeling techniques in place of, or in addition to x-ray crystallography.

In another embodiment, the availability of genetic signature containing nucleic acids enables the production of strains of laboratory mice carrying the signature(s) of the invention. Transgenic mice expressing the genetic signature of the invention provide a model system in which to examine the role of the protein(s) encoded by the signature containing nucleic acid in the development and progression towards ischemic stroke. Methods of introducing transgenes in laboratory mice are known to those of skill in the art. Three common methods include: (1) integration of retroviral vectors encoding the foreign gene of interest into an early embryo; (2) injection of DNA into the pronucleus of a newly fertilized egg; and (3) the incorporation of genetically manipulated embryonic stem cells into an early embryo. Production of the transgenic mice described above will facilitate the molecular elucidation of the role that a target protein plays in various cellular metabolic processes. Such mice provide an in vivo screening tool to study putative therapeutic drugs in a whole animal model and are encompassed by the present invention.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus.

The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extra-chromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring, in fact, possess some or all of that alteration or genetic information, then they, too, are transgenic animals.

The alteration of genetic information may be foreign to the species of animal to which the recipient belongs, or foreign only to the particular individual recipient, or may be genetic information already possessed by the recipient. In the last case, the altered or introduced gene may be expressed differently than the native gene. Such altered or foreign genetic information would encompass the introduction of genetic signature containing nucleotide sequences.

The DNA used for altering a target gene may be obtained by a wide variety of techniques that include, but are not limited to, isolation from genomic sources, preparation of cDNAs from isolated mRNA templates, direct synthesis, or a combination thereof.

A preferred type of target cell for transgene introduction is the embryonal stem cell (ES). ES cells may be obtained from pre-implantation embryos cultured in vitro (Evans et al., (1981) Nature 292:154-156; Bradley et al., (1984) Nature 309:255-258; Gossler et al., (1986) Proc. Natl. Acad. Sci. 83:9065-9069). Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

One approach to the problem of determining the contributions of individual genes and their expression products is to use genetic signature associated genes as insertional cassettes to selectively inactivate a wild-type gene in totipotent ES cells (such as those described above) and then generate transgenic mice. The use of gene-targeted ES cells in the generation of gene-targeted transgenic mice was described, and is reviewed elsewhere (Frohman et al., (1989) Cell 56:145-147; Bradley et al., (1992) Bio/Technology 10:534-539).

Techniques are available to inactivate or alter any genetic region to a mutation desired by using targeted homologous recombination to insert specific changes into chromosomal alleles.

However, in comparison with homologous extra-chromosomal recombination, which occurs at a frequency approaching 100%, homologous plasmid-chromosome recombination was originally reported to only be detected at frequencies between 10⁻⁶ and 10⁻³. Non-homologous plasmid-chromosome interactions are more frequent occurring at levels 10⁵-fold to 10² fold greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformant cells for homologous insertion, followed by screening of individual clones. Alternatively, a positive genetic selection approach has been developed in which a marker gene is constructed which will only be active if homologous insertion occurs, allowing these recombinants to be selected directly. One of the most powerful approaches developed for selecting homologous recombinants is the positive-negative selection (PNS) method developed for genes for which no direct selection of the alteration exists. The PNS method is more efficient for targeting genes which are not expressed at high levels because the marker gene has its own promoter. Non-homologous recombinants are selected against by using the Herpes Simplex virus thymidine kinase (HSV-TK) gene and selecting against its nonhomologous insertion with effective herpes drugs such as gancyclovir (GANC) or (1-(2-deoxy-2-fluoro-B-D arabinofluranosyl)-5-iodou-racil, (FIAU). By this counter selection, the number of homologous recombinants in the surviving transformants can be increased. Utilizing genetic signature containing nucleic acid as a targeted insertional cassette provides means to detect a successful insertion as visualized, for example, by acquisition of immunoreactivity to an antibody immunologically specific for the polypeptide encoded genetic signature nucleic acid(s) and, therefore, facilitates screening/selection of ES cells with the desired genotype.

As used herein, a knock-in animal is one in which the endogenous murine gene, for example, has been replaced with human genetic signature-associated gene(s) of the invention. Such knock-in animals provide an ideal model system for studying the development of acute ischemic stroke.

As used herein, the expression of a genetic signature containing nucleic acid, fragment thereof, or genetic signature fusion protein can be targeted in a “tissue specific manner” or “cell type specific manner” using a vector in which nucleic acid sequences encoding all or a portion of genetic signature-associated protein are operably linked to regulatory sequences (e.g., promoters and/or enhancers) that direct expression of the encoded protein in a particular tissue or cell type. Such regulatory elements may be used to advantage for both in vitro and in vivo applications. Promoters for directing tissue specific expression of proteins are well known in the art and described herein.

Methods of use for the transgenic mice of the invention are also provided herein. Transgenic mice into which a nucleic acid containing the genetic signature or its encoded protein(s) have been introduced are useful, for example, for use in screening methods to identify therapeutic agents modulate or ameliorate the symptoms of ischemic stroke.

### Pharmaceuticals and Peptide Therapies

The elucidation of the role played by the gene products described herein in ischemic stroke occurrence facilitates the development of pharmaceutical compositions useful for the diagnosis, management and treatment of ischemic stroke. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

As it is presently understood, RNA interference involves a multi-step process. Double stranded RNAs are cleaved by the endonuclease Dicer to generate nucleotide fragments (siRNA). The siRNA duplex is resolved into 2 single stranded RNAs, one strand being incorporated into a protein-containing complex where it functions as guide RNA to direct cleavage of the target RNA (Schwarz et al, Mol. Cell. 10:537 548 (2002), Zamore et al, Cell 101:25 33 (2000)), thus silencing a specific genetic message (see also Zeng et al, Proc. Natl. Acad. Sci. 100:9779 (2003)).

Pharmaceutical compositions that are useful in the methods of the invention may be administered systemically in parenteral, oral solid and liquid formulations, ophthalmic, suppository, aerosol, topical or other similar formulations. These pharmaceutical compositions may contain pharmaceutically-acceptable carriers and other ingredients known to enhance and facilitate drug administration. Thus such compositions may optionally contain other components, such as adjuvants, e.g., aqueous suspensions of aluminum and magnesium hydroxides, and/or other pharmaceutically acceptable carriers, such as saline. Other possible formulations, such as nanoparticles, liposomes, resealed erythrocytes, and immunologically based systems may also be used to administer the appropriate agent to a patient according to the methods of the invention. The use of nanoparticles to deliver agents, as well as cell membrane permeable peptide carriers that can be used are described in Crombez et al., Biochemical Society Transactions v35:p 44 (2007).

In order to treat an individual having an acute ischemic stroke, to alleviate a sign or symptom of the disease, the pharmaceutical agents of the invention should be administered in an effective dose. The total treatment dose can be administered to a subject as a single dose or can be administered using a fractionated treatment protocol, in which multiple doses are administered over a more prolonged period of time, for example, over the period of a day to allow administration of a daily dosage or over a longer period of time to administer a dose over a desired period of time. One skilled in the art would know that the amount of agent required to obtain an effective dose in a subject depends on many factors, including the age, weight and general health of the subject, as well as the route of administration and the number of treatments to be administered. In view of these factors, the skilled artisan would adjust the particular dose so as to obtain an effective dose for treating an individual having, or at risk for an acute ischemic stroke.

In an individual suffering from an ischemic stroke, administration of the agent can be particularly useful when administered in combination, for example, with a conventional agent for treating ischemic stroke. The skilled artisan would administer the agent alone or in combination and would monitor the effectiveness of such treatment using routine methods such as sonogram, radiologic, immunologic or, where indicated, histopathologic methods. Other conventional agents for the treatment of ischemic stroke include agents, such as TPA and other anticoagulants. Administration of the pharmaceutical preparation is preferably in an “effective amount” this being sufficient to show benefit to the individual. This amount prevents, alleviates, abates, or otherwise reduces the severity of ischemic stroke symptoms in a patient.

The pharmaceutical preparation is formulated in dosage unit form for ease of administration and uniformity of dosage. Dosage unit form, as used herein, refers to a physically discrete unit of the pharmaceutical preparation appropriate for the patient undergoing treatment.

Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

The following methods are provided in detail to facilitate the practice of the present invention.

### Study Subjects

Peripheral blood samples were obtained from 18 ischemic stroke patients admitted to the University Hospital of Brooklyn at SUNY Downstate Medical Center and at Long Island College Hospital and 15 gender and race matched control subjects recruited from the local community. The median time of blood draw was 36 hours post stroke onset. Stroke was diagnosed according to World Health Organization stroke criteria. The Institutional Review Board at the State University of New York (SUNY) approved the study and all study participants or their authorized representatives gave full and signed informed consent.

The study inclusion criteria were: over 18 years of age and acute ischemic stroke. The exclusion criteria were: current immunological diseases, taking steroid or immunosuppressive therapies, severe allergies, acute infection and severe anemia. The following clinical data were recorded: age, gender, race, self-reported risk factors, National Institutes of Health Stroke Scale (NIHSS) score in the stroke subjects and complete blood counts (CBC), including total white blood cell count and white cell differential counts. Hypertension was defined as a prior (at any time in the past) diagnosis of hypertension by the subject's physician or currently receiving treatment for hypertension. Diabetes was defined as a past medical history of known diabetes mellitus. Coronary artery disease was defined as a physician-diagnosed past history of ischemic heart disease or angina. Hyperlipidemia was defined as a past history of documented elevation in total cholesterol (>200 mg/dl). Smoking was defined as current or prior smoking. Atrial fibrillation was defined as a past or current history of physician-diagnosed atrial fibrillation.

TABLE 1
Clinical and laboratory characteristics of patients and controls

| | All | Stroke | Control | |
|---|---|---|---|---|
| | (n = 33) | (n = 18) | (n = 15) | p |
| Factor | | | | |
| Age | 65.4 ± 14.3 | 71.6 ± 13.0 | 58.1 ± 12.3 | 0.004 |
| Gender-male | 14 (42) | 7 (39) | 7 (47) | 0.9 |
| Race-black | 30 (91) | 17 (94) | 13 (87) | 0.9 |
| Risk factors | | | | |
| Hypertension | 28 (85) | 17 (94) | 11 (73) | 0.2 |
| Diabetes | 15 (45) | 8 (39) | 7 (53) | 0.6 |
| Coronary artery disease | 8 (24) | 5 (28) | 3 (20) | 0.9 |
| Smoking history | 7 (21) | 5 (28) | 2 (13) | 0.6 |
| Atrial fibrillation | 4 (12) | 4 (22) | 0 (0) | 0.2 |
| Hyperlipidemia | 16 (48) | 8 (44) | 8 (53) | 0.9 |
| Medications | | | | |
| Diuretics | 9 (27) | 6 (33) | 3 (15) | 0.6 |
| ACEIs/ARBs | 9 (27) | 7 (39) | 2 (13) | 0.2 |
| Beta blockers | 21 (64) | 14 (78) | 7 (47) | 0.1 |
| Calcium channel blockers | 8 (24) | 5 (28) | 3 (20) | 0.9 |
| Anti-thrombotics | 18 (54) | 10 (55) | 8 (53) | 1.0 |
| Statins | 14 (42) | 7 (39) | 7 (47) | 0.9 |
| WBC count (10⁹ cells/liter) | 6.9 ± 2.4 | 7.45 ± 2.2 | 6.18 ± 2.6 | 0.2 |
| Stroke-Related | | | | |
| Time of blood draw (hours) | N/A | 36.0 (23.0, 48.0) | N/A | N/A |
| Infarct volume (mm³) | N/A | 5404.0 | N/A | N/A |
| | | (1,207.0, 22,870.0)) | | |
| NIHSS score | N/A | 7.5 (4.2, 10.0) | N/A | N/A |

### Primer Selection and Development

40 transcripts identified in 3 previously published studies[4-6] were selected for analysis (Table 2). The 3 studies had identified 9, 18 and 22 genes within panels with some overlap among the studies. Hox 1.11, transcript identified in Tang's et al. study[5], was not studied because it is a non-coding RNA sequence. Hypothetical protein FLJ22662 Laminin A motif from the Moore list[4] is now termed phospholipase B domain containing 1 (PLBD1) according to current nomenclature. Two variants of CD14 were studied to give a total of 41 transcripts that were tested. The complete primer characteristics were published earlier[18]. The RT-qPCR primers were self-designed, commercially synthesized by Invitrogen and wet tested using regular RT-qPCR (StepOnePlus Real-Time PCR Systems; Applied Biosystems).

TABLE 2
Comparison of 41 transcripts between stroke and control subjects

| | Cellular source | Fold | | Adjusted p | Adjusted p |
|---|---|---|---|---|---|
| Transcript | (reference) | change | p value | value* | value** |
| CD163 | PBMC⁴ | 2.22 | 0.069 | 0.14 | 1.0 |
| PLBD1 | PBMC⁴ | 3.18 | 0.0034 | 0.03 | 0.14 |
| ADM | PBMC⁴ | 1.85 | 0.0066 | 0.03 | 0.27 |
| KIAA0146 | PBMC⁴ | 1.21 | 0.43 | 0.52 | 1.0 |
| APLP2 | PBMC⁴ | 1.08 | 0.56 | 0.62 | 1.0 |
| NPL | PBMC⁴, WB⁵ | 1.67 | 0.094 | 0.16 | 1.0 |
| FOS | PBMC⁴ | 2.64 | 0.043 | 0.10 | 1.0 |
| TLR2 | PBMC⁴ | 1.37 | 0.57 | 0.62 | 1.0 |
| NAIP | PBMC⁴ | 1.71 | 0.24 | 0.34 | 1.0 |
| CD36 | PBMC⁴ | 2.11 | 0.29 | 0.10 | 1.0 |
| DUSP1 | PBMC⁴ | 2.89 | 0.033 | 0.10 | 1.0 |
| ENTPD1 | PBMC⁴ | 2.03 | 0.039 | 0.10 | 1.0 |
| VCAN | PBMC⁴, WB⁶ | 2.36 | 0.058 | 0.13 | 1.0 |
| CYBB | PBMC⁴ | 2.61 | 0.0083 | 0.04 | 0.34 |
| IL13RA1 | PBMC⁴ | 1.58 | 0.10 | 0.16 | 1.0 |
| LTA4H | PBMC⁴ | 1.61 | 0.20 | 0.30 | 1.0 |
| ETS2 | PBMC⁴, WB⁵ | 2.86 | 0.017 | 0.07 | 0.70 |
| CD14-1 | PBMC⁴ | 1.93 | 0.065 | 0.14 | 1.0 |
| CD14-2 | PBMC⁴ | 1.39 | 0.74 | 0.78 | 1.0 |
| BST1 | PBMC⁴ | 6.42 | 0.0035 | 0.03 | 0.14 |
| CD93 | PBMC⁴ | 2.11 | 0.00086 | 0.02 | 0.03 |
| PILRA | PBMC⁴ | 1.29 | 0.56 | 0.62 | 1.0 |
| FCGR1A | PBMC⁴ | 3.28 | 0.076 | 0.14 | 1.0 |
| CKAP4 | WB⁵ | 1.93 | 0.0040 | 0.03 | 0.14 |
| S100A9 | WB⁵ | 3.84 | 0.0014 | 0.02 | 0.06 |
| MMP9 | WB^{5,6} | 2.21 | 0.10 | 0.16 | 1.0 |
| S100P | WB⁵ | 2.67 | 0.0399 | 0.10 | 1.0 |
| F5-1 | WB⁵ | 2.14 | 0.034 | 0.10 | 1.0 |
| FPR1 | WB⁵ | 1.79 | 0.07 | 0.14 | 1.0 |
| S100A12 | WB^{5,6} | 2.93 | 0.000593 | 0.02 | 0.02 |
| RNASE2 | WB⁵ | 1.06 | 0.84 | 0.86 | 1.0 |
| ARG1 | WB^{5,6} | 1.34 | 0.34 | 0.42 | 1.0 |
| CA4 | WB^{5,6} | 1.74 | 0.17 | 0.27 | 1.0 |
| LY96 | WB^{5,6} | 1.41 | 0.27 | 0.36 | 1.0 |
| SLC16A6 | WB⁵ | 1.64 | 0.23 | 0.34 | 1.0 |
| HIST2H2AA3 | WB⁵ | 1.48 | 0.25 | 0.34 | 1.0 |
| BCL6 | WB⁵ | 0.97 | 0.58 | 0.62 | 1.0 |
| PYGL | WB⁵ | 2.55 | 0.0059 | 0.03 | 0.24 |
| CCR7 | WB⁶ | 0.995 | 0.96 | 0.96 | 1.0 |
| IQGAP1 | WB⁶ | 1.67 | 0.04 | 0.10 | 1.0 |
| ORM1 | WB⁶ | 1.28 | 0.31 | 0.40 | 1.0 |

*FDR method,
**Bonferroni method
Wilcoxon rank sum tests and t tests used for analyses

TABLE 3
Primers used for amplification of markers

| | | | gene | | | |
|---|---|---|---|---|---|---|
| | | | ID | | | |
| Name | Name | RefSeq | NCBI | Variants | Forward primer | Reverse primer |
| F5 | F5; | NM_000130.4 | 2153 | | AAATCCCATGA | CAGACCCCTAA |
| | coagulation | | | | GTTTCACGCC | CTGGTGCTGTT |
| | factor V | | | | (SEQ ID NO: 1) | (SEQ ID NO: 41) |
| | (proaccelerin, | | | | | |
| | labile factor) | | | | | |
| S100A9 | S100A9; S100 | NM_002965.3 | 6280 | | CTCGGCTTTGAC | TCCCCGAGGCC |
| | calcium | | | | AGAGTGCAAGAC | TGGCTTATGG |
| | binding | | | | (SEQ ID NO: 2) | (SEQ ID NO: 42) |
| | protein A9 | | | | | |
| CD163 | CD163 | NM_004244.4 | 9332 | two variants | GCCACAACAGG | GGCTCAGAATGGC |
| | molecule | | | (2 variants) | TCGCTCATCCC | CTCCTTTTCCA |
| | | | | | (SEQ ID NO: 3) | (SEQ ID NO: 43) |
| TLR2 | toll-like | NM_003264.3 | 7097 | | GCTGCTCGGCGT | TGTCCAGTGCTT |
| | receptor 2 | | | | TCTCTCAGG | CAACCCACAACT |
| | | | | | (SEQ ID NO: 4) | (SEQ ID NO: 44) |
| ENTPD1 | ectonucleoside | NM_001098175.1 | 953 | seven | GCATGCGGTTGC | GGCTCCCCCAA |
| | triphosphate | | | variants (for | TCAGGATGGAAA | GGTCCAAAGC |
| | diphospho- | | | 1, 2, 3, 4, 5: | (SEQ ID NO: 5) | (SEQ ID NO: 45) |
| | hydrolase 1 | | | 254n) | | |
| | (ENTPD1) | | | | | |
| VCAN | versican | NM_001126336.2 | 1462 | four | AAACGACCTGATC | GGCCGCAAGCG |
| | (VCAN), | | | variants | GCTGCAAAATGA | ACTGTTCCTT |
| | | | | (2nd | (SEQ ID NO: 6) | (SEQ ID NO: 46) |
| | | | | variant) | | |
| CD14 | CD14 | NM_001040021.2 | 929 | four | CCCCTTGGTGCC | CGGCTGCCTCTT |
| | molecule | | | variants | AACAGATGAGG | ATATCCCAGAGA |
| | (CD14), | | | (2nd | (SEQ ID NO: 7) | (SEQ ID NO: 47) |
| | | | | variant) | | |
| CD14 | CD14 | NM_001174104.1 | 929 | four | GGTGCCAACAGA | AGCCAGCCCCC |
| | molecule | | | variants | TGAGGTTCACA | TTCCTTTCCTTA |
| | (CD14), | | | (3rd variant) | (SEQ ID NO: 8) | (SEQ ID NO: 48) |
| ADM | adrenomedullin | NM_001124.1 | 133 | | CTTAGCAGGGT | CGAGCGGTGTC |
| | (ADM), | | | | CTGCGCTTCGC | AGCGCCTAG |
| | | | | | (SEQ ID NO: 9) | (SEQ ID NO: 49) |
| DUSP1 | phosphatase 1 | NM_004417.3 | 1843 | | TACGATCAGGG | AGGTGCCTCGG |
| | (DUSP1 | | | | TGGCCCGGTG | TCGAGCACA |
| | | | | | (SEQ ID NO: 10) | (SEQ ID NO: 50) |
| CYBB | cytochrome b- | NM_000397.3 | 1536 | | TCCAGTGCGTGC | TCTGCGGTCTG |
| | 245, beta | | | | TGCTCAACAA | CCCACGTAC |
| | polypeptide | | | | (SEQ ID NO: 11) | (SEQ ID NO: 51) |
| | (CYBB), | | | | | |
| LTA4H | leukotriene | NM_000895.1 | 4048 | | GGGCACCTCTTC | GCAGAGCCGCA |
| | A4 hydrolase | | | | CATTGGGGC | GCCATCTGAA |
| | (LTA4H) | | | | (SEQ ID NO: 12) | (SEQ ID NO: 52) |
| CD36 | CD36 | NM_001001548.2 | 948 | five variants | TCAGCAAATGCAA | GAGGATGACAGG |
| | molecule | | | (1st variant) | AGAAGGGAGACC | AATGCAGGGCC |
| | (thrombo- | | | | (SEQ ID NO: 13) | (SEQ ID NO: 53) |
| | spondin | | | | | |
| | receptor) | | | | | |
| | (CD36 | | | | | |
| NAIP | NLR family, | v1: | 4671 | two variants | ACTGGCCCCGG | TCACCCTGTGC |
| | apoptosis | NM_004536.2; | | (two | GAATCAGCT | CATTTCTGGCA |
| | inhibitory | v2: | | variants) | (SEQ ID NO: 14) | (SEQ ID NO: 54) |
| | protein | NM_022892.1; | | and two | | |
| | (NAIP) | and two | | extra | | |
| | | other products | | products | | |
| APLP2 | amyloid beta | v1: | 334 | four | GGCCGGCTACA | CCGTGTGCCAG |
| | (A4) | NM_001642.2; | | variants (for | TCGAGGCTCT | TGCTGGTGA |
| | precursor-like | v2: | | 1st, 2nd and | (SEQ ID NO: 15) | (SEQ ID NO: 55) |
| | protein 2 | NM_001142276.1; | | 3rd variant) | | |
| | (APLP2) | v3: | | | | |
| | | NM_001142277.1; | | | | |
| FOS | Homo sapiens | NM_005252.3 | 2353 | | CCCCTCCGCTGG | GGTCTGTCTCC |
| | FBJ murine | | | | GGCTTACT | GCTTGGAGTGT |
| | osteosarcoma | | | | (SEQ ID NO: 16) | (SEQ ID NO: 56) |
| | viral | | | | | |
| | oncogene | | | | | |
| | homolog | | | | | |
| | (FOS) | | | | | |
| IL13RA1 | IL13RA1 | NM_001560.2 | 3597 | | GCGCCTACGGAA | TGGTGCTACAC |
| | interleukin 13 | | | | ACTCAGCCACC | TGGGACCCCAC |
| | receptor, | | | | (SEQ ID NO: 17) | (SEQ ID NO: 57) |
| | alpha 1 | | | | | |
| BST1 | BST1 bone | NM_004334.2 | 683 | | TGAGTCCCGAG | GCTGCCTTCCC |
| | marrow | | | | CAGCGGAACAA | CGCAGGATT |
| | stromal cell | | | | (SEQ ID NO: 18) | (SEQ ID NO: 58) |
| | antigen 1 | | | | | |
| CD93 | CD93 CD93 | NM_012072.3 | 22918 | | TGGCAGGCTGG | TCCCCATGGCC |
| | molecule | | | | GTCCCTCTC | CTGGCTTGT |
| | | | | | (SEQ ID NO: 19) | (SEQ ID NO: 59) |
| PILRA | PILRA paired | NM_178272.1 | 29992 | three | AAACTCTCCATC | AGCTGGAGAGG |
| | immunoglobin- | | | variants | ACCCAGGGTCAG | GCAAGGGAAGC |
| | like type 2 | | | (2nd | (SEQ ID NO: 20) | (SEQ ID NO: 60) |
| | receptor alpha | | | variant) | | |
| FCGR1A | FCGR1A Fc | NM_000566.3 | 2209 | | GCAAGTGGACAC | ACCAGGCCTCT |
| | fragment of | | | | CACAAAGGCAG | GCAAGAGCAAC |
| | IgG, high | | | | (SEQ ID NO: 21) | (SEQ ID NO: 61) |
| | affinity Ia, | | | | | |
| | receptor | | | | | |
| | (CD64) | | | | | |
| ETS2 | ETS2 v-ets | NM_005239.4 | 2114 | | CTGGCCGGCTTC | TGGTCCCGGCG |
| | erythro- | | | | ACAGGAAGT | ACCTCAGTC |
| | blastosis virus | | | | (SEQ ID NO: 22) | (SEQ ID NO: 62) |
| | E26 oncogene | | | | | |
| | homolog 2 | | | | | |
| | (avian) | | | | | |
| KIAA0146 | KIAA0146 | NM_001080394.1 | 23514 | | CCGCGCTCGGG | GGTGGTTTCTT |
| | KIAA0146 | | | | GCTCTAAGAG | GCTTGGGTCTA |
| | | | | | (SEQ ID NO: 23) | GGG |
| | | | | | | (SEQ ID NO: 63) |
| PLBD1 | PLBD1 | NM_024829.5 | 79887 | | GGGTTACCTCAC | GCGGAGCAATG |
| | phospholipase | | | | TGCCCCACACAT | TCCCATGTCCC |
| | B domain | | | | (SEQ ID NO: 24) | (SEQ ID NO: 64) |
| | containing 1 | | | | | |
| CKAP4 | cytoskeleton- | NM_006825.3 | 10970 | | CGAGCAGAAGGTG | AGCCGCTCCTC |
| | associated | | | | CAGTCTTTGCAA | CACCGTGTT |
| | protein 4 | | | | (SEQ ID NO: 25) | (SEQ ID NO: 65) |
| MMP9 | matrix | NM_004994.2 | 4318 | | GGGCCGCTCCT | ACCGTCGAGTC |
| | metallo- | | | | ACTCTGCCT | AGCTCGGGT |
| | peptidase 9 | | | | (SEQ ID NO: 26) | (SEQ ID NO: 66) |
| | (gelatinase B, | | | | | |
| | 92 kDa | | | | | |
| | gelatinase, | | | | | |
| | 92 kDa type | | | | | |
| | IV | | | | | |
| | collagenase) | | | | | |
| S100P | S100 calcium | NM_005980.2 | 6286 | | CGATATTCGGG | CTTTTCCACTCT |
| | binding | | | | CAGCGAGGGC | GCAGGAAGCCTG |
| | protein P | | | | (SEQ ID NO: 27) | (SEQ ID NO: 67) |
| FPR1 | formyl | v1: | 2357 | two variants | CCTGAACCTGG | CGGTGCGGTGG |
| | peptide | NM_001193306.1; | | (for two | CCGTGGCTG | TTCTGGGTC |
| | receptor 1 | v2: | | variants) | (SEQ ID NO: 28) | (SEQ ID NO: 68) |
| | | NM_002029.3 | | | | |
| S100A12 | S100 calcium | NM_005621.1 | 6283 | | TTGAGGGGTTAAC | GCAGCCTTCAG |
| | binding | | | | ATTAGGCTGGGA | CGCAATGGC |
| | protein A12 | | | | (SEQ ID NO: 29) | (SEQ ID NO: 69) |
| RNASE2 | ribonuclease, | NM_002934.2 | 6036 | | GCCCCTGAACCC | ACCATGTTTCCC |
| | RNase A | | | | CAGAACAACCA | AGTCTCCGCGC |
| | family, 2 | | | | (SEQ ID NO: 30) | (SEQ ID NO: 70) |
| | (liver, | | | | | |
| | eosinophil- | | | | | |
| | derived | | | | | |
| | neurotoxin) | | | | | |
| ARG1 | arginase, liver | NM_000045.2 | 383 | | GGCGGAGACCAC | GACCTCCCACG |
| | | | | | AGTTTGGCAAT | ACTGGTGTGC |
| | | | | | (SEQ ID NO: 31) | (SEQ ID NO: 71) |
| CA4 | carbonic | NM_000717.3 | 762 | | TCGGCCAGTGC | GGGGGACTGGC |
| | anhydrase IV | | | | AGAGTCACAC | GGTCCTTCT |
| | | | | | (SEQ ID NO: 32) | (SEQ ID NO: 72) |
| LY96 | lymphocyte | v1: | 23643 | two variants | GAGCTCTGAAGG | AAGAGCATTTCT |
| | antigen 96 | NM_015364.4; | | (for all) | GAGAGACTGTGA | TCTGGGCTCCCA |
| | | v2: | | | (SEQ ID NO: 33) | (SEQ ID NO: 73) |
| | | NM_001195797.1; | | | | |
| SLC16A6 | solute carrier | v1: | 9120 | two variants | GGACCGCCCCTT | CACCAGGGCGA |
| | family 16, | NM_001174166.1; | | (for all) | GCAGGTTT | GGCACACAG |
| | member 6 | v2: | | | (SEQ ID NO: 34) | (SEQ ID NO: 74) |
| | | NM_004694.4; | | | | |
| HIST2H2AA3 | histone cluster | AA3 gene: | AA3: | two genes | TTCCCGATCGCC | TTGCCTTTGCG |
| and | 2, H2aa3 and | NM_003516.2; | 8337; | (for two) | AGGCAGGA | CAGCAAGCG |
| HIST2H2AA4 | histone cluster | AA4 gene | AA4: | | (SEQ ID NO: 35) | (SEQ ID NO: 75) |
| | 2, H2aa4 | NM_001040874.1; | 723790 | | | |
| BCL6 | B-cell | NM_001134738.1 | 604 | three | GAAGTGCACGT | GCAACGATAGGG |
| | CLL/lymphoma | | | variants | CCTGCGGCT | TTTCTCACCACA |
| | 6 | | | (3rd variant) | (SEQ ID NO: 36) | (SEQ ID NO: 76) |
| PYGL | phosphorylase, | NM_001163940.1 | 5836 | two variants | CTACGACAAGT | GCTGGATGGCC |
| | glycogen, | | | (2nd | GCCCCAAGCTT | ACCTGATCCG |
| | liver | | | variant) | (SEQ ID NO: 37) | (SEQ ID NO: 77) |
| CCR7 | chemokine | NM_001838.3 | 1236 | | TCCCCAGACAG | CATTGGTTTCCCC |
| | (C-C motif) | | | | GGGTAGTGCG | AGGTCCATGACG |
| | receptor 7 | | | | (SEQ ID NO: 38) | (SEQ ID NO: 78) |
| IQGAP1 | IQ motif | NM_003870.3 | 8826 | | CGCGCCTCCAA | TCCAGGACAGAG |
| | containing | | | | GGTTTCACG | CCATAGTGCGG |
| | GTPase | | | | (SEQ ID NO: 39) | (SEQ ID NO: 79) |
| | activating | | | | | |
| | protein 1 | | | | | |
| ORM1 | orosomucoid | NM_000607.2 | 5004 | | CCAGATACGTGG | GCCCCCAGTTCTT |
| | 1 | | | | GAGGCCAAGAG | CTCATCGTTCA |
| | | | | | (SEQ ID NO: 40) | (SEQ ID NO: 80) |

### Sample Processing

Where applicable the conduct and reporting of the study are in accordance with the Minimum Information for Publication of Quantitative Real-Time PCR Experiments criteria[19]. RNA was extracted using column separation (All-in-One Kit; Norgen Biotek, Thorold, Ontario, Canada) from 100 μl of whole blood collected on ethylenediaminetetraacetic acid tubes (ETDA). Cell count (millions of cells per μl) was based on white blood cell (WBC) count from laboratory CBC obtained for each study subject. cDNA was synthetized using the High Capacity cDNA Reverse Transcription Kit (Life Technologies, Carlsbad, Calif.), based on random hexamers, according to the manufacturer's protocol. In addition to study samples two commercial cDNA samples (Universal cDNA Reverse Transcribed by Random Hexamer: Human Normal Tissues; Biochain, Newark, Calif.) were run on each plate to perform normalization. HT RT-qPCR was run on the BioMark HD System, using 96×96 Fluidigm Dynamic Arrays (Fluidigm, South San Francisco, Calif.). Three plates were used for this study. The percent present calls were over 90%.

### Gene Expression Data Analyses and Development of the Gene Classifier

Gene expression for each sample was measured using the input sample quantity method [20] after adjusting for the input cell count and normalizing to a standard volume of a standard cDNA sample (Universal cDNA Reverse Transcribed by Random Hexamer: Human Normal Tissues; Biochain, Newark, Calif.). The normalized copy number for each sample was obtained according to the equation:

$${X}_{c} = \frac{{( 1 + E )}^{( nCq , cDNA - nCq , X )}}{c c}$$

where X_{c} is the transcript number per cell, E is the efficiency of target cDNA amplification, nCq,cDNA and nCq, X are the cycle number at which amplification crosses the threshold respectively for standard cDNA sample and for sample X, cc is the number of cells used for RNA extraction based on CBC result. The results for the stroke patients and control subjects were then compared. We have identified several predictive clusters, including a 7 gene classifier was identified from a hierarchical cluster analysis. The upper level of normal for the expression of each transcript was defined as a value above the third quartile in the control subjects. We have previously presented graphical results, based only on Cq values normalized to cell count, of four stroke related transcripts in a cohort of hemorrhagic and ischemic stroke patients.

### Statistical Analyses

The data were analyzed using R version 2.15.1. Shapiro's tests were used to assess for normality of the data. For grouped and categorical data t tests, Mann Whiney U, Wilcoxon rank sum and Student's t tests were used to compare groups. Chi-square tests were used to compare categorical values. Spearman correlation coefficients were used to test the association of transcript expression with age and time of blood draw. Corrections for multiple comparisons used the Benjamini and Hochberg (false discovery rate, [FDR]) and Bonferroni algorithms. The hierarchical cluster analysis—a non-supervised technique to detect hidden associations in the data—used Ward's method and log-transformed data. The Ward algorithm employs an Euclidian distance measure. A cutoff (“height”) level at “9” was used to give the 7 Clusters. Receiver operating curve analysis and sensitivity and specificity analyses were used to test diagnostic value of the 7 transcript cluster. p-values <0.05 were considered statistically significant.

The following examples are provided to illustrate certain embodiments of the invention. They are not intended to limit the invention in any way.

### Example 1

### Biomarkers Associated with Acute Ischemic Stroke and Next Generation QPCR and Validation Thereof

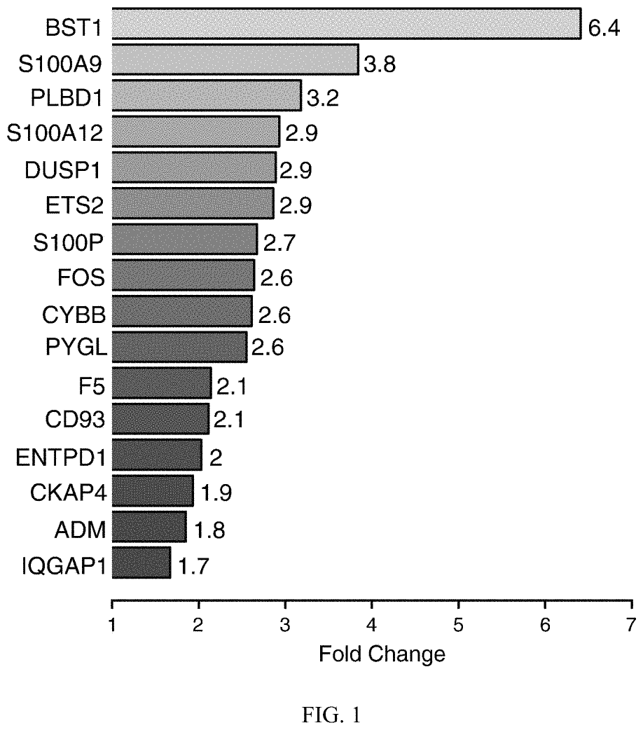

We studied forty candidate markers identified in three gene expression profiles to (1) quantitate individual transcript expression, (2) identify transcript clusters and (3) assess the clinical diagnostic utility of the clusters identified for ischemic stroke detection. Using high throughput next generation qPCR 16 of the 40 transcripts were significantly up-regulated in stroke patients relative to control subjects (p<0.05). Several clusters of between 3 and 8 transcripts discriminated between stroke and control (p values between 1.01×10- and 0.03; Table 4). A 7 transcript cluster containing PLBD1, PYGL, BST1, DUSP1, FOS, VCAN and FCGR1A showed high accuracy for stroke classification (AUC=0.854). A second 7 transcript cluster also proved effective at discriminating between stroke and control patients. This cluster included PILRA, BCL6, FPR1, LYS96, S100A9, S100A12 and MMP9. Moreover, using a panel including the 16 upregulated genes of FIG. 1 achieved p values of 101. The invention also entails analysis of all of the genes listed in Table 4. The invention described herein provides a plurality of validated and improved biomarker panels for diagnosis of acute ischemic stroke, streamlining the diagnostic process and reducing time between presentation and therapeutic intervention at the clinic.

### Results

The patients and controls were matched on gender, race and stroke risk factors (Table 1). The mean age of the stroke patients was 71 years and of the controls was 58 years (p=0.004).

### Whole Blood Expression of Stroke-Related Transcripts.

16 genes were significantly up-regulated in the stroke patients relative to the control subjects (p<0.05, Wilcoxon rank sum test, Table 2). The fold change differences for the 16 transcripts ranged from 6.4 for BST1 to 1.7 for IQGAP1 (FIG. 1). Nine genes were altered at the p<0.01 level: these were CD93, S100A9, CYBB, S100A12, BST1, PLBD1, PYGL, ADM and CKAP4. All of these 9 genes remained significant after corrections for multiple comparisons using the FDR method and two (S100A12 and CD93) using the Bonferroni method. 41% (9/22) of genes from the PBMC list were significantly altered. 38% (8/21) from the whole blood gene lists were significantly altered, 7 genes were on Tang et al. list and 2 were on Barr et al. list. One transcript was common to both the whole blood and PBMC lists (this was ETS2) and one transcript was common to both WB lists (S100A12). Although, modest correlations with age for two transcripts were identified—FOS (rho=0.42, p=0.02) and PYGL (rho=0.43, p=0.02), after corrections for multiple comparisons these correlations were no longer significant. There was no correlation of transcript copy number with gender or with the time of blood draw.

### Clusters of Genes in Whole Blood

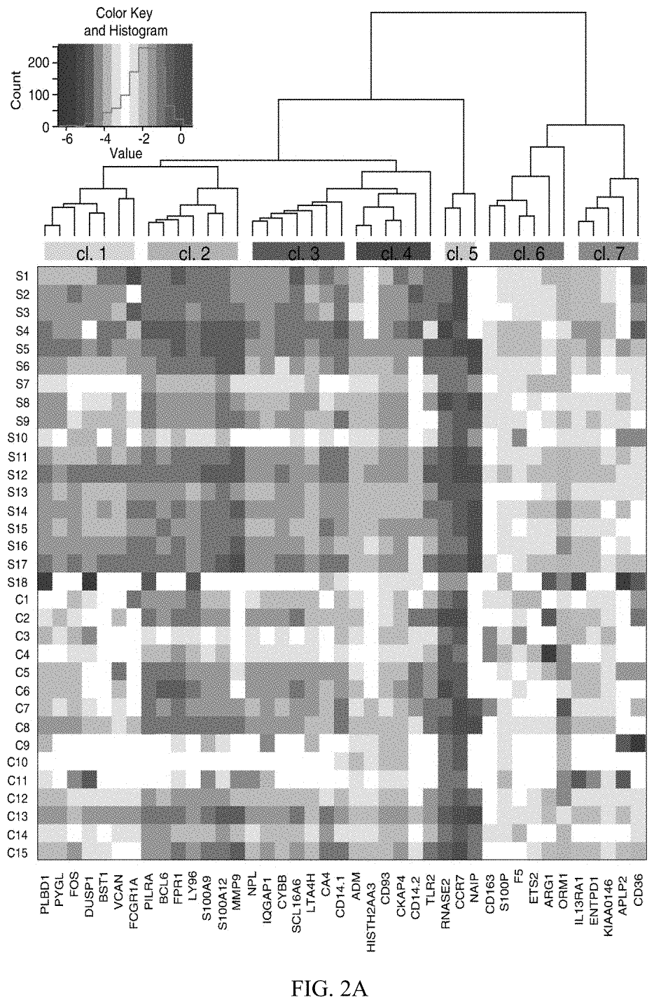

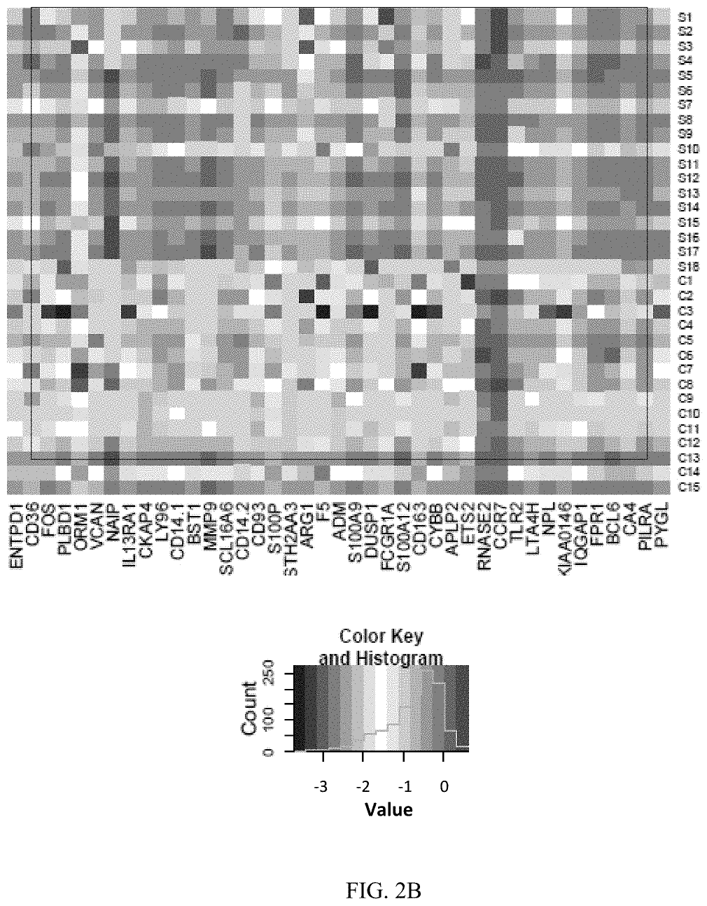

Several clusters of between 3 and 7 transcripts were identified in a hierarchical cluster analysis. (FIG. 2A). Six of these showed significant discrimination between stroke and control with p values for the six clusters ranging between 1.10×1-⁻⁹ for Cluster 1 and 0.037 for Cluster 7 (Table 4). After correction for multiple comparisons using the Bonferroni method all but one remained significant. The clusters consisted of transcripts from both whole blood and PBMC studies. Based on the demonstration of the most significant discrimination between stroke and control Cluster 1 was selected for further study.

TABLE 4
Transcript clusters identified in hierarchical cluster analysis

| | Cellular | | | |
|---|---|---|---|---|
| | sources | P value, of | | |
| | (number of | cluster, stroke | Adjusted | Adjusted p |
| Transcripts | genes) | versus control | p value* | value** |
| Cluster 1 | PBMC panel | 1.01e−9 | 7.04e−9 | 7.04e−9 |
| 7 genes | (5), both | | | |
| PLBD1, PYGL, FOS, | PBMC and | | | |
| DUSP1, BST1, | WB panels (1), | | | |
| VCAN, FCGR1A | WB panel (1) | | | |
| Cluster 2 | WB panels (6), | 1.50e−6 | 5.26e−6 | 1.05e−5 |
| 7 genes | PBMC panel | | | |
| PILRA, BCL6, FPR1, | (1) | | | |
| LY96, S100A9, | | | | |
| S100A12, MMP9 | | | | |
| Cluster 3 | PBMC panel | 1.52e−5 | 3.55e−5 | 1.06e−4 |
| 7 genes | (3), PBMC and | | | |
| NPL, IQGAP1, | WB panels (1), | | | |
| CYBB, SLC16A6, | WB panels (3) | | | |
| LTA4H, CA4, CD14-1 | | | | |
| Cluster 4 | PBMC panel | 0.0016 | 0.0022 | 0.011 |
| 6 genes | (4), WB panel | | | |
| ADM, HIST2H2AA3, | (2) | | | |
| CD93, CKAP4, | | | | |
| CD14-2, TLR2 | | | | |
| Cluster 5 | WB panels (2), | 0.40 | 0.40 | 1.0 |
| 3 genes | PBMC panel | | | |
| NAIP, RNASE2, | (1) | | | |
| CCR7 | | | | |
| Cluster 6 | WB panels (4), | 0.00025 | 4.0e−4 | 1.61e−3 |
| 6 genes | PBMC panel | | | |
| CD163, S100P, F5, | (1), WB and | | | |
| ETS2, ARG1, ORM1 | PBMC panels | | | |
| | (1) | | | |
| Cluster 7 | PBMC panel | 0.037 | 0.042 | 0.26 |
| 5 genes | (5) | | | |
| APLP2, IL13RA1, | | | | |
| ENTPD1, KIAA0146, | | | | |
| CD36 | | | | |
| Cluster 8 | PBMC Panel | 4.7 × 10⁻¹¹ | | |
| 8 genes | and WB Panel | | | |
| PLBD1, PYGL, | | | | |
| BST1, DUSP1, FOS, | | | | |
| NPL, KIAA0146, | | | | |
| ENTPD1 | | | | |
| Cluster 9 | PBMC Panel | 8.2 × 10⁻⁸ | | |
| 6 genes | and WB Panel | | | |
| IL1RA1, ADM, | | | | |
| HIST2HAA, CD93, | | | | |
| CA4, CYBB | | | | |
| Cluster 10 | PBMC panel | .002 | | |
| 3 genes | | | | |
| CD36, VCAN, | | | | |
| FCGR1A | | | | |
| Cluster 11 | WB Panel | 5 × 10⁻⁷ | | |
| 6 genes | | | | |
| IQGAP1, LTA4H, | | | | |
| SLC16AC, CD14-2, | | | | |
| CD14-1, CKAP4 | | | | |
| Cluster 12 | WB Panels | 5 × 10⁻⁷ | | |
| 7 genes | | | | |
| F5, S100P, CD163, | | | | |
| ETS2, ORM1, ARG, | | | | |
| APLP2 | | | | |

Wilcoxon rank sum tests used for analyses,
*FDR.
**Bonferroni

### Performance of a 7 Gene Cluster for Stroke Classification

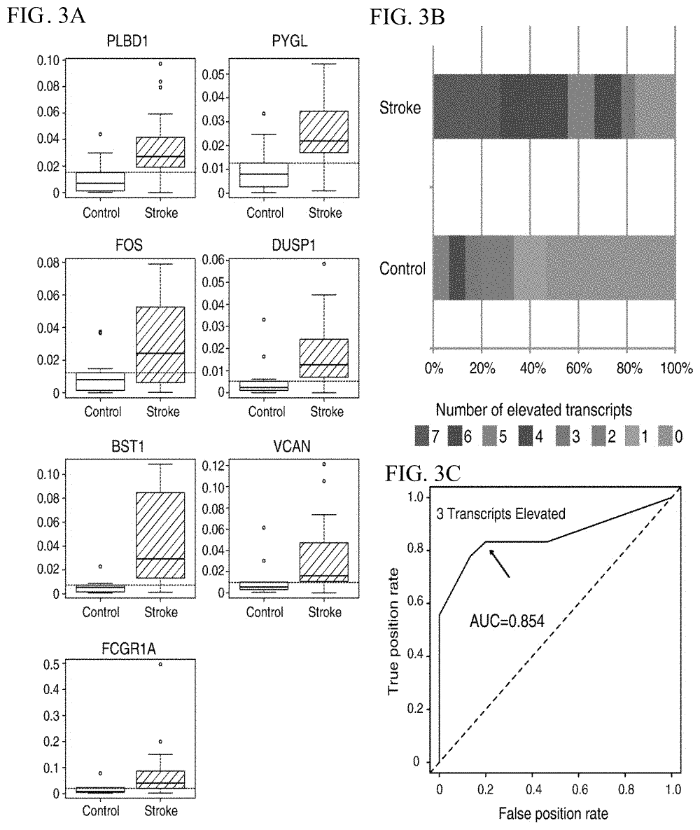

The 7 transcript Cluster 1 consisted of PLBD1, PYGL, BST1, DUSP1, FOS, VCAN and FCGR1A (Table 4 and Table 5). Five of these transcripts had differed significantly between stroke and control. The upper threshold levels for each of the transcripts were based on the third quartile in the control subjects (Table 4 and FIG. 3A). Absent calls were noted for BST1 and FCGR1A in a number of the control subjects (this could reflect low or absent transcript expression). The number of subjects with elevated expression of each transcript is shown in

TABLE 5
The proportion of patients with elevated expression of between 0-7 transcripts is shown
in Figure 3B.

| | | | Control | |
|---|---|---|---|---|
| | | | Number of | |
| | | Stroke | subjects with | |
| | | Number of subjects | elevated | |
| | | with elevated | transcript copy | |
| | | transcript copy | number | |
| Transcript | Threshold | number (%) | (%) | p |
| PLBD1 | >0.0144 | 15/18 (83%) | 3/14 (21%) | 0.0017 |
| PYGL | >0.0115 | 13/17 (76%) | 3/12 (25%) | 0.02 |
| FOS | >0.0122 | 10/17 (59%) | 3/13 (23%) | 0.11 |
| DUSP1 | >0.0052 | 13/17 (76%) | 2/11 (18%) | 0.008 |
| BST1 | >0.0073 | 14/16 (88%) | 2/7 (28%) | 0.02 |
| VCAN | >0.0101 | 12/17 (70%) | 3/9 (33%) | 0.16 |
| FCGR1A | >0.0202 | 10/14 (71%) | 2/6 (33%) | 0.27 |
| 7 transcripts in | 3 or more | 15/18 (83%) | 3/15 (20%) | 0.001 |
| Cluster 1 | transcripts elevated | | | |

Elevated whole blood expression of at least 3 transcripts in this 7 gene cluster classified stroke with a sensitivity of 83% and a specificity of 80% (FIG. 3B). The overall accuracy of the 7 gene classifier was high (AUC=0.854, FIG. 3C).

Performance of Three Previously Reported Transcript Panels The Moore et al. transcripts list[4], identified in PBMCs, showed a highly significant discrimination between stroke and control (p=1.01e-9). The p values for the Tang et al. list[5] and the Barr et al. list[6], identified in whole blood, were 1.05e-5 and 0.02 respectively.

### Discussion

The diagnostic utility of gene expression changes in acute ischemic stroke has been studied in a number of prior microarray studies[4-7,21,22]. However these microarray results were never validated with qPCR—the gold standard for measuring gene expression. The current study was based on three studies where gene panels had been identified using the Prediction Analysis for Microarrays[4-6] algorithm. The Grond-Ginsbach et al. study[7] was not included as only one transcript was identified and pooled samples were used. Results from Oh et al.[21] were published after this study commenced. In several other microarray studies the utility of gene expression was investigated: for the evaluation of the risk of hemorrhagic transformation[23], defining stroke etiology[24-26] and in studying gender related gene expression changes in stroke patients[27,28].

Using HT RT-qPCR, for the first time—a new qPCR based platform that has the advantages of high accuracy and sensitivity—we have found that 40% of the transcripts were up-regulated in stroke. It is arguable as to whether corrections for multiple comparisons were needed as these transcripts were apriori specified. Nevertheless even after correction for multiple comparisons expression of a small number of transcripts were still significantly different between stroke and control. Although, the hierarchical cluster analysis was not used previously it proved to be very successful in detecting association of studied transcripts with stroke. This analysis grouped genes into 7 clusters. These clusters were highly significantly different between the stroke patients and the control subjects, with 5 remaining highly significant after stringent correction for multiple comparisons (p values as low as 7.04e-9). The cluster of 7 genes—PLBD1, PYGL, BST1, DUSP1, FOS, VCAN and FCGR1A—classified stroke with high sensitivity and specificity, respectively 80% and 83%. The similar expression of genes within a cluster in the stroke patients and control subjects, with comparable differences between two groups permitted the analysis of the expression of all genes within a cluster together. Furthermore, the quantitative information on copy number permitted threshold levels of normal and abnormal expression to be established in the control subjects.

Of interest is that while whole blood samples were used in this study, the 16 significantly altered transcripts had been identified in whole blood and in PBMCs, and that transcripts within each of the 7 identified clusters came from both whole blood and PBMC gene lists. These two cell populations overlap substantially because whole blood is composed of PBMC and polymorphonuclear leukocytes (granulocytes). Neutrophils are the main cell population within polymorphonuclear leukocytes and represent the most numerous nucleated cell fraction in whole blood, however their RNA content is almost three times lower than in PBMC[18]. The overlap between panels and detection of PBMC gene alterations in whole blood samples supports the validity of the microarray results.

Accurate and rapid stroke diagnosis is crucial for timely and effective treatment in the acute phase. Diagnosis is also necessary in subacute and delayed phase to evaluate future risks and for optimal prevention strategies. Timely diagnosis is absolutely essential for treating patients with tissue plasminogen activator—the only FDA approved treatment of ischemic stroke.

However, this treatment improves the chances of recovering from stroke only if administered within 3 to 4.5 hours. Stroke diagnosis may not be conclusive in the acute phase of stroke, especially in stroke mimics, while in subacute or chronic stroke, silent stroke, inconclusive brain imaging or atypical stroke presentation may confound stroke diagnosis. Hence, additional tests, such as molecular tests, that could confirm a diagnosis of stroke, or add complementary information, are much needed.

Molecular diagnostic tests based on gene expression patterns are now available in number of diseases, including breast, colon, lung, prostate and thyroid cancers. These tests have been based on microarray identified panels and detection of overall signal intensities rather than measurements of the expression of individual genes[14,29,30]. For stroke diagnosis, methods that are highly complex, labor intensive and require expensive equipment are difficult to be applied to clinical practice and need to be available rapidly. HT RT-qPCR is very promising and can address this problem[18]. HT RT-qPCR permits absolute quantification and measuring gene expression adjusted to the input cell count, is independent of control genes. An HT RT-qPCR identified classifier may be used to develop a point-of-care system for stroke diagnosis. Until now stroke gene expression panels established in microarray studies consisted of 18 to 79 genes[4,5,22]. Clusters of 5-7 genes established using HT RT-qPCR are more feasibly applied in clinical setting, where short turnaround times and low detection limits are crucial. We have recently discussed the requirements of this system and provide a highly sensitive gene expression profiling method in Example 2 that can be measured almost in real time[15].

In summary, a proportion of previously reported genes in microarray studies in stroke were replicable using HT RT-qPCR and all except 3 were grouped together to form gene clusters highly significant for ischemic stroke detection. Grouping genes in clusters allowed the identification of gene expression classifiers that could be used in a point-of-care system. These results show the promise and potential for continuing studies of gene expression profiling in stroke and for further assessment of the sensitivity and specificity of transcript clusters for ischemic stroke detection and diagnosis. Further studies will examine the gene expression changes in terms of cellular source, time course and relation to clinical outcome.

### REFERENCES FOR EXAMPLE 1

[1] V. L. Feigin, M. H. Forouzanfar, R. Krishnamurthi, et al., Global and regional burden of stroke during 1990-2010: findings from the Global Burden of Disease Study 2010, Lancet. 6736 (2013) 1-11.
[2] Á. Chamorro, A. Meisel, A. M. Planas, et al., The immunology of acute stroke, Nat. Rev. Neurol. 8 (2012) 401-410.
[3] C. Iadecola, J. Anrather, The immunology of stroke: from mechanisms to translation, Nat.

Med. 17 (2011) 796-808.
[4] D. F. Moore, H. Li, N. Jeffries, et al., Using peripheral blood mononuclear cells to determine a gene expression profile of acute ischemic stroke: a pilot investigation, Circulation. 111 (2005) 212-221.
[5] Y. Tang, H. Xu, X. Du, et al., Gene expression in blood changes rapidly in neutrophils and monocytes after ischemic stroke in humans: a microarray study, J. Cereb. Blood Flow Metab. 26 (2006) 1089-1102.
[6] T. L. Barr, Y. Conley, J. Ding, et al., Genomic biomarkers and cellular pathways of ischemic stroke by RNA gene expression profiling, Neurology. 75 (2010) 1009-1014.
[7] C. Grond-Ginsbach, M. Hummel, T. Wiest, et al., Gene expression in human peripheral blood mononuclear cells upon acute ischemic stroke, J. Neurol. 255 (2008) 723-731.
[8] R. L. VanGilder, J. D. Huber, C. L. Rosen, T. L. Barr, The transcriptome of cerebral ischemia, Brain Res. Bull. 88 (2012) 313-319.
[9] F. R. Sharp, G. C. Jickling, Whole genome expression of cellular response to stroke, Stroke. 44 (2013) S23-S25.
[10] A. E. Baird, Blood genomics in human stroke, Stroke. 38 (2007) 694-8.
[11] F. R. Sharp, G. C. Jickling, B. Stamova, et al., RNA expression profiles from blood for the diagnosis of stroke and its causes, J. Child Neurol. 26 (2011) 1131-1136.
[12] P. T. Nelson, D. A. Baldwin, L. M. Scearce, et al., Microarray-based, high-throughput gene expression profiling of microRNAs, Nat. Methods. 1 (2004) 155-161.
[13] E. Wang, L. D. Miller, G. A. Ohnmacht, et al., High-fidelity mRNA amplification for gene profiling, Nat. Biotechnol. 18 (2000) 457-459.
[14] S. Singhal, D. Miller, S. Ramalingam, S.-Y. Sun, Gene expression profiling of non-small cell lung cancer, Lung Cancer. 60 (2008) 313-324.
[15] Z. Peng, B. Young, A. E. Baird, S. A. Soper, Single-pair fluorescence resonance energy transfer analysis of mRNA transcripts for highly sensitive gene expression profiling in near real time, Anal. Chem. 85 (2013) 7851-7858.
[16] S. Palmer, A. P. Wiegand, F. Maldarelli, et al., New real-time reverse transcriptase-initiated PCR assay with single-copy sensitivity for human immunodeficiency virus type 1 RNA in plasma, J. Clin. Microbiol. 41 (2003) 4531-4536.
[17] A. S. Devonshire, R. Sanders, T. M. Wilkes, et al., Application of next generation qPCR and sequencing platforms to mRNA biomarker analysis, Methods. 59 (2013) 89-100.
[18] M. G. Adamski, Y. Li, E. Wagner, et al., Next-generation qPCR for the high-throughput measurement of gene expression in multiple leukocyte subsets, J. Biomol. Screen. 18 (2013) 1008-1017.
[19] S. A. Bustin, V. Benes, J. A. Garson, et al., The MIQE guidelines: minimum information for publication of quantitative real-time PCR experiments, Clin. Chem. 55 (2009) 611-622.
[20] M. G. Adamski, P. Gumann, A. E. Baird, A Method for Quantitative Analysis of Standard and High-Throughput qPCR Expression Data Based on Input Sample Quantity, PLoS One. 9 (2014) e103917.
[21] S.-H. Oh, O.-J. Kim, D.-A. Shin, et al., Alteration of immunologic responses on peripheral blood in the acute phase of ischemic stroke: blood genomic profiling study, J. Neuroimmunol. 249 (2012) 60-65.
[22] B. Stamova, H. Xu, G. Jickling, et al., Gene expression profiling of blood for the prediction of ischemic stroke, Stroke. 41 (2010) 2171-2177.
[23] G. C. Jickling, B. P. Ander, B. Stamova, et al., RNA in blood is altered prior to hemorrhagic transformation in ischemic stroke, Ann. Neurol. (2013). DOI: 10.1002/ana.23883
[24] G. C. Jickling, B. Stamova, B. P. Ander, et al., Profiles of lacunar and nonlacunar stroke, Ann. Neurol. 70 (2011) 477-485.
[25] G. C. Jickling, H. Xu, B. Stamova, et al., Signatures of cardioembolic and large-vessel ischemic stroke, Ann. Neurol. 68 (2010) 681-692.
[26] H. Xu, Y. Tang, D.-Z. Liu, et al., Gene expression in peripheral blood differs after cardioembolic compared with large-vessel atherosclerotic stroke: biomarkers for the etiology of ischemic stroke, J. Cereb. Blood Flow Metab. 28 (2008) 1320-1328.
[27] B. Stamova, Y. Tian, G. Jickling, et al., The X-chromosome has a different pattern of gene expression in women compared with men with ischemic stroke, Stroke. 43 (2012) 326-334.
[28] Y. Tian, B. Stamova, G. C. Jickling, et al., Effects of gender on gene expression in the blood of ischemic stroke patients, J. Cereb. Blood Flow Metab. 32 (2012) 780-791.
[29] E. K. Alexander, G. C. Kennedy, Z. W. Baloch, et al., Preoperative diagnosis of benign thyroid nodules with indeterminate cytology, N. Engl. J. Med. 367 (2012) 705-715.
[30] C. Sotiriou, M. J. Piccart, Taking gene-expression profiling to the clinic: when will molecular signatures become relevant to patient care?, Nat. Rev. Cancer. 7 (2007) 545-553.

### Example 2

### A Method for Quantitative Analysis of Standard and High-Throughput qPCR Expression Data Based on Input Sample Quantity

Over the past decade a rapid increase has occurred in the understanding of RNA expression and its regulation. Quantitative polymerase chain reaction(s) (qPCR) have become the gold standard for measuring gene expression. Accurate analysis of qPCR data is crucial for optimal results and a number of well-defined methods are in use to calculate gene expression. These include the comparative C_{T} method [1], the efficiency corrected method [2] and sigmoidal curve fitting methods [3], all of which provide relative quantitative information. A standard curve of serial dilutions of a known sample is additionally required to measure the absolute number of transcript copies in a sample.

For most scientific purposes, relative quantification, expressed as fold change, is sufficient to provide the required information. Hence, the comparative C_{T} and efficiency corrected methods, as well as the sigmoidal curve fitting methods are widely employed, but each method has strengths and weaknesses. The comparative CT method by Livak et al. [1] has the advantage of ease of use but is based on the assumption that transcript amplification efficiencies are 100%. In the efficiency corrected method by Pfaffl [2] the relative expression ratio is calculated only from the real-time PCR efficiencies and the crossing point deviation of an unknown sample versus a control. This model needs no calibration curve and gives improved quantification but is complex to use and requires determination of the amplification efficiency.

Furthermore, all of these methods require the use of reference (control or housekeeping) genes to correct for unequal amounts of biological material that may exist between the tested samples. The commonly used housekeeping genes were initially selected on the basis of their abundance and expression in a wide variety of tissues. An absolute requirement and widely held assumption of housekeeping genes has been that their expression is constant under all conditions and is unaffected by the experimental conditions [4]. However, the expression of commonly used housekeeping genes has since been found to vary considerably in many conditions [5-12]. In the case of in vitro or ex-vivo experiments it is usually possible to perform additional experiments to identify and validate appropriate control genes. In the case of clinical studies, however, where sample volumes are usually limited, it is rarely possible to test gene expression before and after the experiment (i.e., before and after the disease occurs).

The advent of next generation high throughput qPCR, based on reaction volumes scaled to the nanoliter range and with a consequent dramatic reduction in the volume of reagents and samples, has been a major advance for the analysis of clinical samples [13]. The Fluidigm Biomark system, one of the new high-throughput reverse transcription PCR (HT RT-qPCR) systems, permits up to 96 transcripts in 96 samples to be studied simultaneously during a single run, in a total of 9216 reactions. This allows many more transcripts to be studied from routine clinical samples, representing a 40 to 50 fold improvement in efficiency over standard qPCR [14,15]. However, HT RT-qPCR has also raised new issues; for example, transcript amplification efficiency may be affected by potential interactions (i.e., primer dimer, competition) between multiple primers during the preamplification and amplification steps.

In the present example, a method for the measurement of the absolute gene expression for standard and high throughput qPCR experiments based on the input sample quantity is described. Based on this method three equations were developed: (1) for the measurement of fold change differences between target and control samples; (2) for the comparison of results from different experiments and different machines after normalization to a reference cDNA sample; (3) for analyses of samples of unknown efficiency. Gene expression results calculated using the input quantity method were then validated in a serial dilution series of commercial cDNA and using different starting cell concentrations. In clinical samples, fold change values calculated with the input quantity method were compared to values obtained using other commonly used algorithms. The input quantity method has the advantages of avoiding the use of control genes, of being efficiency corrected, and providing both fold change and absolute results. This method can also be applied in the verification and quantification of qualitative results from microarray studies for multiple genes.

### Requirements for the Input Quantity Method

The input quantity method has several requirements. First, the amount of material used for RNA extraction has to be measured: for example, cell count is required for cell suspensions (e.g., peripheral blood mononuclear cells (PBMCs), lymphocytes and cell lines), white blood cell (WBC) counts are needed for whole blood studies and tissue volumes are needed for solid tissues. Secondly, for reverse transcription of RNA to cDNA the same reagents, volumes and protocols for a given experiment need to be used. Thirdly, the amplification efficiency and correlation coefficients (R²) should be assessed for each gene assay based on a standard dilution series. Finally, full application of this method requires the use of a standard sample (i.e., commercial cDNA—reverse transcribed cDNA from RNA extracted from all human tissues) for each measurement.

Mathematical Model for qPCR Amplification

As per Livak et al.[1], in the qPCR target cDNA sequence is amplified in an exponential fashion:

Xₙ=X₀×(1+E)ⁿ [1]

where Xₙ is the number of target cDNA molecules after n cycles, X₀ is the number of cDNA molecules before amplification, E is the efficiency of target cDNA amplification and n is the number of amplification cycles. In the case of perfect efficiency (E=100/o) the number of target cDNA molecules doubles every cycle.

In qPCR, the number of target cDNA molecules for a given sample is reflected by the threshold cycle—or according to the MIQE guidelines[4], quantification cycle (Cq)—because Cq is the intersection between an amplification curve and threshold. The threshold is the level of fluorescence above background fluorescence—set at the same level for all samples in the experiment. Each sample that crosses the threshold (regardless of the amplification cycle number) has the same fluorescence intensity hence the same target cDNA copy number.

X_{nCq}=X₀×(1+E)^{nCq}=K [2]

where XnCq is the number of target cDNA molecules at the Cq, nCq is the cycle number at which amplification crosses the threshold and K is a constant value for all samples in a given experiment.

### Analysis Normalized to Input Sample Quantity

In order to adjust the results of gene expression to unequal amounts of starting material the number of cells used for RNA extraction has to be incorporated into Equation 2.

X₀=X_{c}×cc [3]

where Xc is the transcript number per cell and cc is the number of cells used for RNA extraction (e.g., complete blood count for whole blood analysis, or hemocytometer cell count for cell subset analysis). Hence,

K=(X_{c}×cc)×(1+E)^{nCq} [4]

Therefore to compare gene expression between target (T) and control (C) samples where E and K are the same for T and C, ccT is the input cell count for target sample and ccC is the cell input for the control sample. For the target samples the following formula is obtained:

K=(T_{C}×ccT)×(1+E)^{nCq,T} [5]

where T_{C} is the number of transcripts per cell in the target samples.
For the reference or control samples the following formula is obtained

K=(C_{c}×ccC)×(1+E)^{nCq,C} [6]

where C_{C} is the number of transcripts per cell in the reference samples.
As K is constant, Equations 4 and 5 equal each other:

(T_{C}×ccT)×(1+E)^{nCq,T}=(C_{C}×ccC)×(1+E)^{nCq,C} [7]

To obtain the comparison between target and control samples:

$$\frac{{T}_{c}}{{C}_{c}} = \frac{c c C}{c c T} \times {( 1 + E )}^{( nCq , C - nCq , T )} \qquad [ 8 ]$$

This way we can obtain the measure of gene expression expressed as a fold change difference between the test and control samples.
Analysis Normalized to Input Quantity and Normalized to Standard cDNA

When a standard reference sample is introduced, for example a sample that contains a high concentration of studied transcripts, the following modifications are made, starting with Equation 2, K for sample X with a starting quantity of cc is:

K=(X_{C}×cc)×(1+E)^{nCq,X} [9]

K for a standard cDNA of uniform quantity is:

K=cDNA₀×(1+E)^{nCq,cDNA} [10]

Normalizing to cDNA:

$$\begin{matrix}( {X}_{c} \times ccX ) \times {( 1 + E )}^{nCq , X} = c D N {A}_{0} \times {( 1 + E )}^{nCq , cDNA} & [ 11 ] \\ {X}_{c} = c {DNA}_{0} \times \frac{{( 1 + E )}^{( nCq , cDNA - nCq , X )}}{c c X} & [ 12 ]\end{matrix}$$

Since the number of transcripts before amplification in standard cDNA (cDNA₀) is constant we may assume it is equal to 1 then:

$${X}_{c} = \frac{{( 1 + E )}^{( nCq , cDNA - nCq , X )}}{c c X} \qquad [ 13 ]$$

To obtain the comparison between test and control samples, the respective T_{c} and C_{c} are calculated using Equation 13. Then Tc is divided by Ce to obtain the measure of gene expression, expressed as a fold change.

Analysis Normalized to Input Quantity and/or Normalized to Standard cDNA without Known Efficiency

If E for the working primers is not assessed in the experiment, one may make an assumption that the E equals 100%-then Equation 8 is:

$$\frac{{T}_{0}}{{C}_{0}} = \frac{c c , C}{c c , T} \times {2}^{( nCq , C - nCq , T )} \qquad [ 14 ]$$

Whereas, adjusting to the standard cDNA sample, for sample X Equation 12 is:

$${X}_{c} = \frac{{2}^{( nCq , cDNA - nCq , X )}}{c c X} \qquad [ 15 ]$$

The following materials and methods are provided to facilitate the practice of the invention described below.

To assess the reliability of the input quantity method, the stability of expression values calculated across serial dilutions of a standard cDNA sample and of different starting numbers of two samples of peripheral blood mononuclear cells (PBMCs) were determined. The validity of the input quantity method was assessed by comparison to fold changes obtained using the Livak [1] and Pfaffl [2] methods for three transcripts in a cohort of stroke patients and control subjects.

The Institutional Review Board at the State University of New York (SUNY) Downstate Medical Center approved the study. All study participants and/or authorized representatives gave full and signed informed consent. Where applicable, the conduct and reporting of the study are in accordance with the MIQE criteria[4]. The detailed laboratory protocols but not the data analysis described in this manuscript have been previously published [14].

### RNA Extraction and Reverse Transcription

Whole blood was obtained from 38 ischemic stroke patients between 7 and 90 days post stroke and from 17 sex- and race-matched control subjects. RNA was extracted using column separation (All-in-One Kit; Norgen Biotek, Thorold, Ontario, Canada) from 100 μl of whole blood and from a median of 2.0 million CD4⁺ cells. Peripheral blood mononuclear cells (PBMCs) from two control subjects were used for the cell dilution experiment, with RNA isolated from triplicate samples of 2 million, 1 million, 0.5 million and 0.25 million cells. Cellular counts (millions of cells per μl) were measured using a hemocytometer for CD4⁺ and for PBMCs; for whole blood, the total white blood cell count was obtained from the laboratory-measured complete blood count (CBC) in each study subject.

Density gradient centrifugation with Histopaque 1077 and 1119 (Sigma-Aldrich, St. Louis, Mo.) was used to separate the PBMC fraction from the whole blood. Positive magnetic bead separation (Miltenyi Biotec, Bergisch Gladbach, Germany) was used to separate CD4⁺ from PBMCs—the cellular purity was over 97%. The extracted RNA was resuspended in 50 μl of elution solution (All-in-One Kit protocol). cDNA was synthetized using the High Capacity cDNA Reverse Transcription Kit (Life Technologies, Carlsbad, Calif.), based on random hexamers, according to the manufacturer's protocol. Following the protocol, the proportion of RNA solution to 2× RT master mix was 1:1.

Primer Development, RT qPCR and HT-RT qPCR

The primers for qPCR were self-designed, commercially synthesized by Invitrogen and wet tested using standard RT qPCR (StepOnePlus Real-Time PCR Systems; Applied Biosystems).

Standard RT qPCR (StepOnePlus Real-Time PCR Systems; Applied Biosystems) was used to measure the expression of FDFT1 in the cell dilution experiment. Each sample and no template control were measured in triplicate. Based on a standard dilution series the efficiency for FDFT1 in this experiment was 94%.

HT RT-qPCR was run on the BioMark HD System, using 96×96 Fluidigm Dynamic Arrays (Fluidigm, South San Francisco, Calif.). HT-RT qPCR was used first, to measure the expression of FUT4, CD3E, FDFT1 and B2M in serial dilutions of commercial cDNA (Universal cDNA Reverse Transcribed by Random Hexamer: Human Normal Tissues; Biochain, Newark, Calif.) and second, to compare the expression of FDFT1, CD3E and B2M between control subjects and stroke patients in whole blood and CD4⁺ T lymphocytes. Two 5 point, four-fold serial dilution series of commercial cDNA were run in triplicate on two different plates. The volumes of commercial cDNA (diluent) in each dilution were: 100 μl (1:1), 25 μl (1:4), 6.25 μl (1:16), 1.5625 μl (1:64) and 0.39 μl (1:256). According to the manufacturer's protocol, the assay for each HT RT-qPCR experiment contained 10 μl of cDNA. The efficiencies for the genes, assessed with HT RT-qPCR, were: B2M—87%, FDFT1—86%, FUT4—79% and CD3E—79%. Five separate gene expression plates were used in this experiment. To normalize the gene expression results for stroke and control samples from different plates, a sample of commercial cDNA (containing high concentrations of all of the transcripts studied) of standard concentration and volume was run in duplicate on each plate. Each raw gene expression result (expressed as Cq) was normalized to the average Cq value for the same gene in the commercial cDNA samples that were run on the same plate (sample Cq value for gene X was subtracted from the average commercial cDNA Cq for gene X).

### Calculation of Fold Changes

Fold change differences between stroke patients and control subjects for B2M and CD3E were calculated using the input sample quantity method according to Equation 13. The relative gene expression for B2M and CD3E were measured using the comparative CT method of Livak et al.[1] and the efficiency corrected method of Pfaffl[2]. For these calculations FDFT1 was used as control gene as its expression was not different in stroke patients compared to control subjects, based on the input quantity method (p>0.05).

Statistical analyses The statistical analyses were performed using “R”, version 2.15.2. For the cDNA dilution analysis, linear regression modeling was used. For the cell dilution series, the data were analyzed using one way ANOVA, Welch's correction for inhomogeneity of variances and post hoc t.tests with false discovery rate correction. For the analysis of the stroke versus control data, the 95% CI for the fold change values were calculated using the R package “mratios” and Dunnetts method; Wilcoxon rank sum tests were used for between group comparisons.

Gene Expression Measurements Across Different Input Volumes of a Standard cDNA Sample

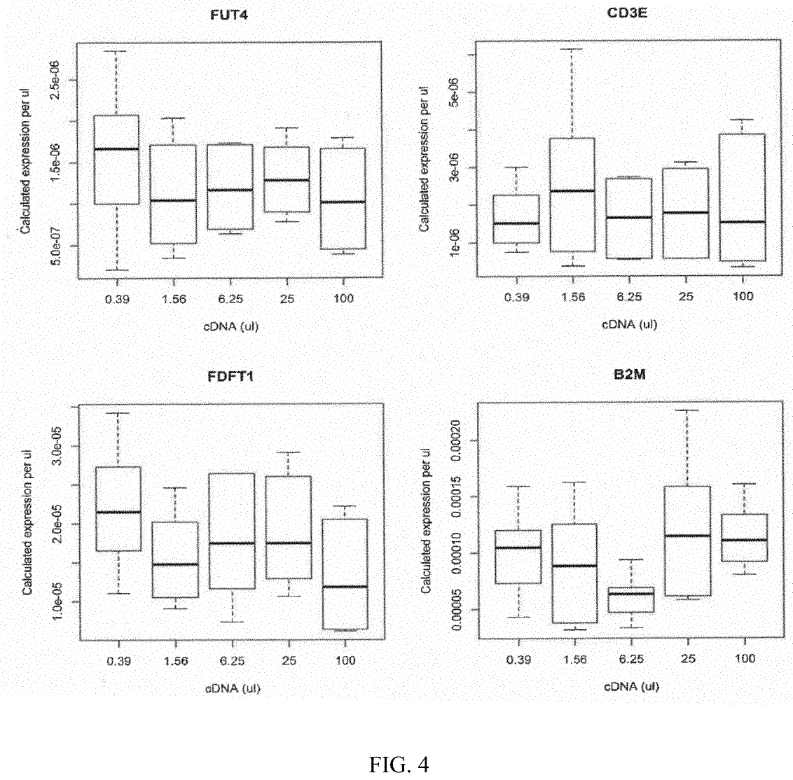

To confirm the reliability of the sample input quantity method, the expression of 4 transcripts (FUT4, CD3E, FDFT1 and B2M) was measured in 5 point and 4-fold two serial dilutions of a standard cDNA sample. To measure the concentrations of each of the four transcripts in the standard cDNA sample, the results were normalized to the volume of diluent 100 μl (1:1), 25 μl (1:4), 6.25 μl (1:16), 1.5625 μl (1:64) and 0.39 μl (1:256). Using this normalization procedure the same expression values were expected across the range of dilutions of the standard cDNA sample. The samples were run in triplicate on two separate plates giving 6 readings per input volume. The expression of all four genes calculated with the input quantity method was stable (Table 6, FIG. 4).

TABLE 6
Expression of FUT4, CD3E, FDFT1 and B2M across serial volumes
of a standard cDNA sample.

| | FUT4 | CD3E | FDFT1 | B2M |
|---|---|---|---|---|
| Coefficient | −4.3e−8 | −6.43e−8 | −3.8e−7 | −3.3e−6 |
| P value | 0.49 | 0.65 | 0.61 | 0.48 |
| R² | −0.018 | −0.028 | −0.026 | −0.016 |

Dilution coefficient, p and R² values were obtained from linear regression analysis for each transcript

### Reliability of Gene Expression Measurements Across Different Starting Numbers of Cells

In order to determine the influence of variables present prior to the RT qPCR step (cell counting, RNA isolation and RT PCR) the expression of FDFT1 in different starting numbers of PBMCs from two control subjects was measured. The raw data were normalized to the starting number of cells for each subject. The starting numbers of cells (2 million, 1 million, 0.5 million and 0.25 million) were within the range of the manufacturer's recommendations for RNA extraction (All-in-One Kit, Norgen Biotec).

Based on the input quantity method the expression of FDFT1 was significantly different across the input cell counts for both subjects (p=1.4e-7, Subject 1 and p=5.5e-5, Subject 2) (Table 7). Post hoc tests revealed that the expression of FDFT1 in the 0.25 million input cell count in both subjects differed significantly from the other input cell concentrations: in Subject 1 (versus 2 million, p=2.7e-6, versus 1 million, p=0.00016 and versus 0.5 million, p=7.6e-5) and in Subject 2 (versus 2 million, p=5.9e-5, versus 1 million, p=1.3e-6 and versus 0.5 million, p=1.7e-6). Comparisons between the 2 million, 1 million and 0.5 million input cell numbers were not statistically significant for both subjects (p<0.05).

TABLE 7
Expression of FDFT1 in cell dilution series

| | | | 0.5 million | 0.25 million | |
|---|---|---|---|---|---|
| | 2 million cells | 1 million cells | cells | cells | p |
| Subject 1 | 0.26 ± 0.01 | 0.23 ± 0.02 | 0.24 ± 0.06 | 0.15 ± 0.02** | <<0.01 |
| Subject 2 | 0.049 ± 0.003 | 0.041 ± 0.005 | 0.043 ± 0.015 | 0.072 ± 0.013** | <<0.01 |

p values were calculated using a one-way ANOVA.
**Post hoc tests revealed that expression of FDFT1 in the 0.25 million input cell count differed significantly from the other input cell concentrations in both subjects.

### Expression of CD3E and B2M in the Late Phase of Stroke and in Control Subjects Calculated Using Three Methods

To assess the validity of the input quantity method using clinical samples, the expression of CD3E and B2M in whole blood and in CD4⁺ T lymphocytes was compared between patients in the delayed phase of stroke and control subjects. Fold change differences in gene expression were measured using the input quantity method (normalized to cell count), and the Livak and Pfaffl methods.

By all methods B2M expression was significantly increased in whole blood in the delayed phase of stroke and CD3E was significantly increased in CD4 cells (Table 8). No alterations in the expression of CD3E were found in whole blood. A borderline increased in B2M expression in CD34 cells was found using the input quantity method.

TABLE 8
Fold change difference in the expression of B2M and CD3E in late phase
stroke versus control subjects.

| | B2M | | | CD3E | | |
|---|---|---|---|---|---|---|
| | Input | | | Input | | |
| | Quantity | B2M | B2M | Quantity | CD3E | CD3E |
| | Method | Livak | Pfaffl | Method | Livak | Pfaffl |
| Whole | | | | | | |
| blood | | | | | | |
| Fold | 2.51 | 2.19 | 2.28 | 1.27 | 1.12 | 1.22 |
| Change | | | | | | |
| 95% CI | 1.26, 15.89 | 1.26, 5.94 | 1.32, 6.20 | 0.67, 3.34 | 0.70, 2.01 | 0.79, 2.07 |
| p | 0.017 | 0.006 | 0.003 | 0.19 | 0.48 | 0.42 |
| CD4 | | | | | | |
| Fold | 1.35 | 0.57 | 0.70 | 3.13 | 1.78 | 2.10 |
| Change | | | | | | |
| 95% CI | 0.94, 2.17 | 0.26, 1.15 | 0.41, 1.23 | 1.61, 25.8 | 1.16, 3.42 | 1.35, 4.25 |
| p | 0.02 | 0.4999 | 0.26 | 2.10e−05 | 0.0084 | 7.50e−05 |

### Discussion

Several gene expression analysis methods are in common use, but the input quantity approach presented here offers two major advantages. Firstly, this method is independent of control genes. Secondly, with the assumptions of 1) uniform efficiency of RNA extraction and RT qPCR and 2) a constant concentration and volume of a standard sample, this method permits absolute quantification, expressed as the fraction of transcripts in the standard sample, across different experiments. The proposed algorithm is efficiency corrected, although analysis of results without known efficiency is also possible. With the use of a standard sample, the input quantity method also permits the comparison and analysis of results from different batches and results acquired on different qPCR machines. Furthermore, with the advent of HT RT-qPCR, this analytical method is also very useful for clinical research, where sample volumes are limited.

Our analyses show that the sample input quantity method permits gene expression to be measured across a wide range of commercial cDNA. Although the performance of both RNA extraction and RT qPCR may differ significantly across different cell concentrations and kits[15], our results show that, using the same protocol and reagents within the input quantities we tested, these variables can be successfully controlled. Furthermore, the expression of B2M and CD3E in study subjects calculated using three methods was highly concordant.

The rationale for the use of housekeeping (or control or reference genes) is to correct gene expression results, reflected as differences in Cq values between target and control samples, that could result from two main factors: different amounts of starting material or different levels of expression. Traditionally, housekeeping genes have been chosen on the basis of their abundance, ubiquitous expression across tissues and the assumption that their expression is stable under physiological and experimental conditions. However, the expression of conventionally used housekeeping genes varies considerably in many conditions. Therefore, reference gene selection requires additional experiments to validate gene expression stability under different experimental conditions[6-12,14]. In many conditions, especially in the clinical setting, it is not possible to measure the effect of the disease/condition on reference gene expression.

The algorithm used for our sample input quantity method employs normalization to the sample input quantity (cell count, tissue volume etc.), which in result permits an absolute gene expression analysis. This method varies from the relative analysis approach, where results are normalized to reference gene expression. Due to normalization to the input quantity (measured in absolute scale) the measure of gene expression remains absolute, as in our method. In contrast, the gene expression from the relative analysis approach is based on the normalization to reference gene expression. Thus the ratio of the target gene expression to the reference gene expression represents a relative measure. By introducing a standard sample (of a stable transcript concentration), our method allows us to compare gene expression between different experiments. Instead of directly measuring transcript copy number—as it is commonly done in absolute measurements of gene expression—in our method, the measured gene expression is presented as a fraction of transcripts present in the standard sample. This fraction can be converted to the transcript copy number by measuring concentration of the target gene in the standard sample.

The input quantity approach presented here can be applied to clinical studies, to verify and quantitate microarray results, and to large scale studies of gene or microRNA expression. Having knowledge of the input cell count for all samples and the use of a uniform standard, first, allows normalization to the amount of starting material, and second, the use of the same standard allows normalization of results between different laboratories and different equipment.

### REFERENCES FOR EXAMPLE 2

1. Livak K J, Schmittgen T D (2001) Analysis of relative gene expression data using real-time quantitative PCR and the 2(−Delta Delta C(T)) Method. Methods 25: 402-408.
2. Pfaffl M W (2001) A new mathematical model for relative quantification in real-time RT-PCR. Nucleic Acids Res 29: e45.
3. Liu M, Udhe-Stone C, Goudar C T (2011) Progress curve analysis of qRT-PCR reactions using the logistic growth equation. Biotechnol Prog 27: 1407-1414.
4. Bustin S a, Benes V, Garson J a, Hellemans J, Huggett J, et al. (2009) The MIQE guidelines: minimum information for publication of quantitative real-time PCR experiments. Clin Chem 55: 611-622.
5. Dheda K, Huggett J F, Bustin S a, Johnson M a, Rook G, et al. (2004) Validation of housekeeping genes for normalizing RNA expression in real-time PCR. Biotechniques 37: 112-119.
6. Vandesompele J, De Preter K, Pattyn F, Poppe B, Van Roy N, et al. (2002) Accurate normalization of real-time quantitative RT-PCR data by geometric averaging of multiple internal control genes. Genome Biol 3: RESEARCH0034.
7. Chang T J, Juan C C, Yin P H, Chi C W, Tsay H J (1998) Up-regulation of beta-actin, cyclophilin and GAPDH in NiS1 rat hepatoma. Oncol Rep 5: 469-471.
8. Feroze-Merzoug F, Berquin I M, Dey J, Chen Y Q (2002) Peptidylprolyl isomerase A (PPIA) as a preferred internal control over GAPDH and beta-actin in quantitative RNA analyses. Biotechniques 32: 776-782.
9. Nishimura M, Nikawa T, Kawano Y, Nakayama M, Ikeda M (2008) Effects of dimethyl sulfoxide and dexamethasone on mRNA expression of housekeeping genes in cultures of C2C12 myotubes. Biochem Biophys Res Commun 367: 603-608.
10. Lin J, Redies C (2012) Histological evidence: housekeeping genes beta-actin and GAPDH are of limited value for normalization of gene expression. Dev Genes Evol 222: 369-376.
11. Sikand K, Singh J, Ebron J S, Shukla G C (2012) Housekeeping gene selection advisory: glyceraldehyde-3-phosphate dehydrogenase (GAPDH) and β-actin are targets of miR-644a. PLoS One 7: e47510.
12. Li R, Shen Y (2013) An old method facing a new challenge: re-visiting housekeeping proteins as internal reference control for neuroscience research. Life Sci 92: 747-751.
13. Devonshire A S, Sanders R, Wilkes T M, Taylor M S, Foy C a, et al. (2013) Application of next generation qPCR and sequencing platforms to mRNA biomarker analysis. Methods 59: 89-100.
14. Adamski M G, Li Y, Wagner E, Yu H, Seales-Bailey C, et al. (2013) Next-generation qPCR for the high-throughput measurement of gene expression in multiple leukocyte subsets. J Biomol Screen 18: 1008-1017.
15. Spurgeon S L, Jones R C, Ramakrishnan R (2008) High throughput gene expression measurement with real time PCR in a microfluidic dynamic array. PLoS One 3: e1662.

### Example 3

### Rapid Diagnostic Tests for Acute Ischemic Stroke Using Extracellular Vesicles as a Source for mRNA

We hypothesized that EVs released by CD8(+) T-cells contain gene profiles similar to their host cells after an inflammatory response, and thus could be used to detect AIS. We isolated EVs derived from activated CD8(+) T-cells via affinity enrichment using a microfluidic device, named an EV micro-affinity purification (EV-MAP) device. While EVs are commonly isolated using differential centrifugation¹⁵ with a recovery as low as 10%¹⁶, it requires 5-12 h to complete. Precipitation techniques with polyethylene glycol (PEG) decrease processing time.¹⁷ However, all EVs emanating from different biological cells are isolated and not just the disease-associated ones, which can mask mRNA expression differences associated with AIS onset.¹⁸

### Materials and Methods

Materials and reagents. Materials and reagents included COC cover plates (6013S-04) and substrates (5013L-10) (TOPAS Advanced Polymers), reagent-grade IPA (isopropyl alcohol), 1-ethyl-3-[3-dimethylamino-propyl] carbodimide hydro-chloride (EDC), N-hydroxysuccinimide (NHS), 2-(4-morpholino)-ethane sulfonic acid (MES), BSA, Triton X-100, polyvinylpyrrolidone, 40 kDa (PVP-40), Histo-paque 1077, and PEG secured from Sigma-Aldrich. Phosphate buffered saline (PBS, pH=7.4), RPMI-1640 medium, and FBS were purchased from Gibco laboratories. Sodium dodecyl sulfate (SDS), Micro-90, LIVE/DEAD Kit, and Toluidine Blue O were obtained from Fisher Scientific. TBS, Tween20, and EVA Green supermix were obtained from Bio-Rad. Other reagents included uranyl acetate (Polysciences), TapeStation supplies (Agilent), LPS from Escherichia coli 0111:B4 (LPS) (InvivoGen), Zymo RNA kit (Zymo Research), BCA Protein Assay Kit (Pierce), ProtoScript II First Strand cDNA Synthesis Kit (New England Bio-Labs). Antibodies used in these studies included anti-human CD8α mAbs (Clone #37006), APC conjugated mouse anti-human CD8α mAb (Clone #37006), FITC-labeled anti-CD45 mAb, and APC conjugated mouse IgG2B anti hCD8α Ab obtained from R&D systems/Biotechne.

Cell line and growth conditions. MOLT-3 T-cells (ATCC CRL-1552) were grown in RPMI-1640 medium supplemented with 10% FBS. The cells were grown at 37° C. in 5% CO2. To remove the EVs from FBS, the serum was ultracentrifuged (LM8 Beckman Coulter Ultracentrifuge) at 100,000×g for 18 h. EV-depleted FBS was used in the cell culture media. Cell viability was monitored using a LIVE/DEAD kit.

T-cell stimulus with LPS. To model stroke conditions in cells, we stimulated MOLT-3 T-cells with LPS. The stock solution of LPS was prepared in sterile PBS. Then, the cells (culture started with approximately one million cells) were cultured with LPS and monitored for morphology changes and viability at time points up to 75 h. Control experiments were carried out without stimulating the cells with LPS. At each time point, cell viability was evaluated using the LIVE/DEAD viability/cytotoxicity kit for mammalian cells.

Determination of protein concentration. Protein concentration was evaluated with the BCA Protein Assay Kit (Pierce) according to the manufacturer's protocol. Calibration curves were constructed between 0.5 and 100 μg/mL BSA concentration (y=0.0745x+0.0936, R²=0.998). Protein samples were collected in the following way: RIPA Lysis buffer (1×) was infused into the EV-MAP at 10 μL/min and ˜150 μL of lysate was collected. The solutions were immediately sampled by the BCA assay. Usually 5 μL of the sample was mixed with 95 μL of water and 100 μL of kit components (solution A, solution B, and solution C at a volumetric ratio of 25:24:1, respectively). The samples were incubated for 60 min at 60° C. Following reaction, the absorption spectra for standards and samples were collected using a UV-Vis spectrophotometer (Shimadzu) using absorption at a peak maximum of 560 nm.

EV precipitation with PEG. PEG precipitation of EVs was carried out as previously reported.²⁹ In brief, the procedure was as follows: plasma sample was mixed in 0.5× volume of PBS and mixed with 2 mg/mL proteinase K. Proteins were digested for 20 min at 37° C. An equal volume of PEG was added to the mixture of plasma, PBS, and proteinase K. After adding PEG, the tube was inverted and placed in 4° C. overnight before centrifuging the solution at 4000×g for 1 h at 4° C. The pellet was lysed (vortexed thoroughly to dissolve the pellet completely) and RNA was isolated using the Zymo RNA kit according to the manufacturer's protocol.

Microfluidic fabrication. Three-bed EV-MAP devices were produced via hot embossing into COC from a molding master fabricated in brass via high-precision micromilling (HPMM). Seven-bed EV-MAP devices used in this study were fabricated in COP via injection molding (Stratec, Austria) from a mold insert made via UV-LiGA⁴⁰.

Diffusion dynamics in the Monte Carlo simulations of the EV-MAP device. The dynamics of EV affinity-selection can be split into two separate events: (1) Delivery of EVs from solution to the device's surface where the capture antibodies are located; and (2) binding of the surface-bound Ab to the EV. The efficiencies of both processes dictate device recovery. We developed a Monte Carlo fluid dynamics simulation incorporating chemical physics and fluid dynamic principles to guide the design of micropillar-based devices. Previously, we outlined renditions of these chemical physics for CTC affinity-selection and diffusion models for the affinity-selection of labeled membrane proteins, although the diffusion model presented herein is a significant advancement compared to our previous reports.

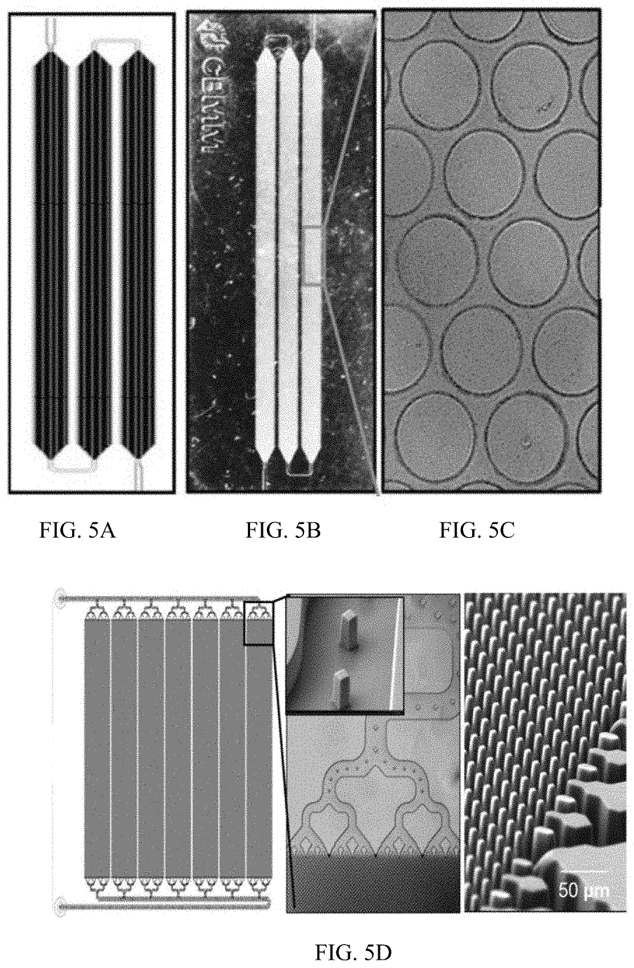

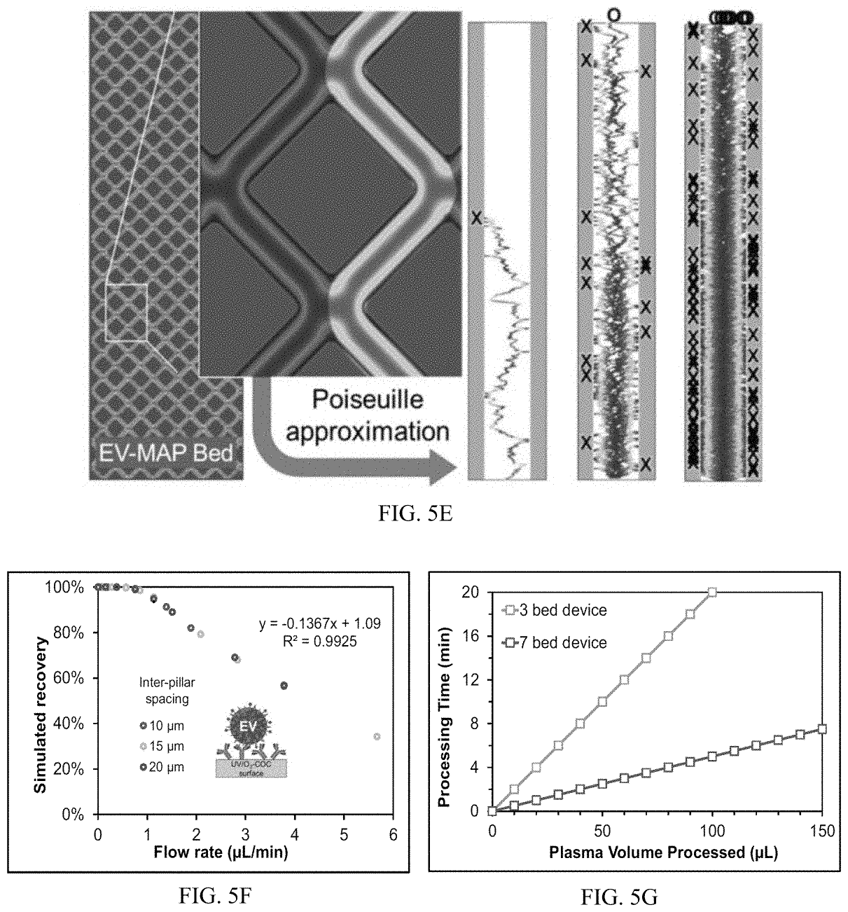

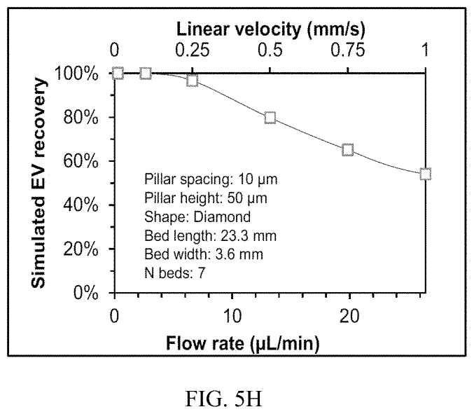

As can be seen in FIG. 5F, the inter-pillar space was modeled as a linear fluidic channel with a constant width and depth determined by the pillar spacing and pillar height, respectively. For diamond-shaped pillars, this spacing is constant and as such, the linear velocity when operated under a constant volume flow rate is the same irrespective of location in the pillared bed as noted in FIG. 5H.

The delivery of EVs to the antibody-coated surface is limited by diffusion through the plasma matrix. As an EV is hydrodynamically transported through the device, it diffuses laterally and longitudinally according to Fick's Second Law of diffusion. Over a small time increment, Δt, the probability that an EV will diffuse a distance x_{D} from its initial position is given by a Gaussian distribution, P(x):

$$P ( x ) = \frac{1}{σ \sqrt{2 π}} {e}^{- \frac{{x}_{D}^{2}}{2 {σ}^{2}}} \qquad ( Eq . S1 )$$

This Gaussian distribution has a standard deviation given by σ=\sqrt{2DΔt}, where D is the EV's diffusion coefficient. Thus, smaller EVs with higher D are more likely to diffuse further in the time interval Δt.

In addition to diffusive transfer, the EVs experience Poiseuille flow. In a high aspect ratio microchannel with a width of W, the EV's forward velocity at position x from the channel's midline can be approximated by:

$$V ( x ) = 1.5 {V}_{a v e} ( 1 - {( \frac{x}{W / 2} )}^{2} \qquad ( Eq . S2 )$$

In Eq. S2, Vₐᵥₑ is simply calculated by dividing the volumetric flow rate by the channel's cross-sectional area. The consequences of the parabolic flow profile in Eq. S2 are complex. As the EV diffuses closer to the surfaces, the EV forward motion slows, and more time is given for diffusion to occur. Consequently, the residence time of two EVs within the same device will not be the same if they take different random paths through the device.

We used a Monte Carlo approach to simulate the flow path of individual EVs through our pillared devices. This process was repeated until the average recovery converged to a constant value. This model allowed us to test various device bed lengths, inter-pillar spacings (W), and average flow velocities (Vₐᵥₑ) to design architectures with high recovery, high throughput, and high surface areas.

For each EV, the Monte Carlo simulation propagates an EV's axial position (x dimension) and longitudinal position (y dimension) over finite time steps (Δt):

x(t)=x(t−Δt)+rand(P(σ(D,Δt)) (Eq. S3a)

y(t)=y(t−Δt)+V(x(t−Δt))+rand(P(σ(D,Δt)) (Eq. S3b)

In Eq. S3a, the EV's lateral x position changes with axial diffusion over Δt using rand(P(σ(D, Δt)), which is given by a pseudo-random number generator that moves the EV laterally according to a Gaussian P(x) distribution with standard deviation σ. Longitudinal diffusion was considered in the same manner by Eq. S3b, but the EV had an additional term due to Poiseuille flow, namely the V(x(t−Δt)) term described in Eq. S2.

mAb-binding dynamics in the Monte Carlo simulations of EV-MAP recovery. As an EV diffuses to and interacts with the device's surfaces, successful binding between the surface-bound mAb and the transient EV is not guaranteed in a single encounter. In general, multiple encounters are necessary for successful EV/mAb binding. Herein, we adopted the Chang-Hammer model to describe this process.

The Chang-Hammer model describes the binding process between surface-confined mAbs and transient antigens, such as those present on the membrane of an EV. This model considers mAb-antigen binding kinetics, the transient motion of the antigen and its associated residence time in proximity to the surface-confined Ab, and the distance over which the EV rolls along the surface.

Previously, we reduced the Chang-Hammer model to a few key equations, and herein, we adopted these dynamics.

First, as the EV rolls along the device's surface, the forward rate constant kₒ for an encounter of antigens with a surface-confined mAb is:

kₒ=2aᵢV_{eff} (Eq. S4)

In Eq. S4, aᵢ is the mAb-antigen interaction radius (2 nm), and V_{eff} is the velocity of the antigen relative to the surface, which is roughly half (0.47) the rolling EV's velocity due to the opposing rotational motion produced by the EV's surface. Furthermore, as the antigen encounters the mAb, the probability that they complex (P) is a function of both the mAb's binding kinetics, kᵢₙ, and the encounter duration;

$$\begin{matrix}P = \frac{{k}_{i n}}{{k}_{i n} + 1 / τ} & ( Eq . S5 ) \\ τ = 8 {a}_{i} / 3 π {V}_{eff} & \end{matrix}$$

As the EV's linear velocity increases, τ decreases, yielding less time available for the mAb and antigen to complex, and P decreases as well. Both the encounter rate, kₒ, and the binding probability, P, are weighted against one another to yield an effective forward rate constant, k_{f}:

k_{f}=kₒP (Eq. S6)

Lastly, the overall rate of EV adhesion k_{ad} combines k_{f} with the EV's antigen surface density, C_{∞};

k_{ad}=k_{f}C_{∞} (Eq. S7)

To review, k_{ad} considers the EV's antigen expression and the velocity of the EV's antigens, both in terms of how often the antigens encounter mAbs and how probable a binding event will occur given the balance of antigen-mAb interaction time and the mAb's binding kinetics. To relate k_{ad} to experimental parameters, consider an EV rolling along a mAb-coated surface at a linear velocity (V) for only a limited distance (L). The percent of EVs that will bind is:

$${\%}_{b o u n d} = 1 - 1 / {e}^{\frac{{k}_{a d} L}{V}} \qquad ( Eq . S8 )$$

Two aspects of Eq. S8 that improve EV recovery are immediately apparent: (i) Decrease the linear velocity; and (ii) maximize the interaction length between the EV and the surface. Unlike CTC dynamics, EVs have a relatively higher diffusion coefficient and the dynamics of an EV rolling along the micropillar surface are associated with a Peclet number <1. Very little can be done to counter lateral diffusion and control or manipulate the length of any given EV-micropillar interaction. Further, changing the bulk flow rate will do little to effect Eq. S8, which only describes the velocity at the surface, because surface flow velocities are limited to approximately zero by the no-slip condition. The velocity in Eq. S8 is more likely to be affected by EV diffusion rather than fluid velocity. Thus, the probability of mAb-binding is dictated by the binding dynamics of the affinity-agent and external manipulation of the device's processing parameters (decreasing fluid velocity, decreasing inter-pillar spacing), which largely affect the diffusion-based delivery of EVs to the surface.

Implementation of Physical Dynamics into Monte Carlo Model and Model Validation.

The flow profile through the device bed with length L_{bed} experienced by an EV was approximated as a straight microfluidic channel with a width W equal to the interpillar spacing and length L=L_{bed}C, where C is a correction factor linked to elongation of the flow path due to the pillar's geometry. For diamond micropillars, C=\sqrt{2}≈1.41, and for circular micropillars, C=π/2≈1.57.

EVs were initiated at 11 positions along the pseudo-channel's midline, and the EV's position through the channel was propagated by using Eqs. S3a and S3b. If the EV encounters the channel's surface (x=±W/2), the EV is propagated by multiplying V(x) (Eq. S2) by the simulation's time step Δt, and the probability of binding was calculated via Eqs. S4-S8. The binding probability was turned into an actionable decision (i.e., binding or not) by using a pseudo-random number generator uniformly distributed between 0 and 1. If the random number was less than Eq. S8's binding probability, the EV was recovered. If not, the EV's position was propagated further via Eqs. S3a and S3b. This series of events continued until either the EV was recovered or the EV was lost (y=L).

Each EV's track is a binary event, recovered or lost, and thousands of EVs were tracked until the simulated recovery converges, defined herein as a <0.01% change in average recovery when additional EVs were tracked. An additional convergence criterion stipulated a <10% standard deviation for five repetitive simulations. Lastly, given various Vₐᵥₑ were tested, the program's discretization of time into Δt time steps was added as a final convergence criterion; after halving the Δt increment, the averaged solution from five simulations must differ by <1% else the simulations would be repeated after halving Δt again.

The accuracy of the Monte Carlo program was first tested by removing all recovery effects and letting EVs freely diffuse; the analytical model of Fick's Second Law (Eq. S1) then becomes fundamentally valid. The results from the Monte Carlo simulation agreed well with a Gaussian function produced via Eq. S1 (FIG. 5F). After enabling device recovery but without Chang-Hammer dynamics, where any surface interaction was considered successful, EVs accumulated along the channel for a total recovery of 64%. The Chang-Hammer dynamics governing the probability of mAb-EV binding in Eqs. S4-S8 were then activated (axial distribution not shown) and the device recovery dropped substantially to 16% for this set of simulation parameters; inter-pillar spacing of 10 μm but short bed length of 2.5 mm and average velocity of 1 mm/s, which reduced the overall time available for axial diffusion.

Lastly, we compared the Monte Carlo model to our previous, less precise model for the set of experimental data in Battle, et al.⁴² Our previous simulation, which did not take into account Chang-Hammer dynamics and did not couple diffusion with fluid flow, generated 68% recovery for membrane proteins, while the Monte Carlo method predicted 75% recovery, which better approached the experimental values of 90±2%. Further improvements to the model, namely improving the Poiseuille approximation (Eq. S2) to better approximate the flow profile around a pillar would include reduced flow velocities between pillar rows and increase residence time available for diffusion and overall recovery in the model.

Immobilization of mAbs. Modification of COC and COP devices for affinity selection of cells and EVs employed a single-stranded oligonucleotide bifunctional cleavable linker containing a uracil residue that could be cleaved using a USER@²⁴. Concentrations of anti-CD8α mAb were 1 mg/mL and 2 mg/mL for the 3-bed and 7-bed EV-MAP devices, respectively. Detailed procedures for mAb immobilization were reported previously²⁴.

T-cells and EV affinity purification. CD8(+) T-cells or CD8(+) MOLT-3 cells were isolated from healthy donor whole blood and cell media, respectively, using curvilinear channel devices modified with anti-human CD8α antibody²². The blood samples were collected into EDTA tubes to prevent the coagulation of blood and were analyzed on the same day the blood was collected. Two milliliters of MOLT-3 cells (˜1×106 cells/mL) was centrifuged for 10 min at 300× g to pellet the cells. The cell pellet was resuspended in 5 mL of PBS, and 1 mL of the suspension was infused onto a cell isolation chip. Both CD8(+) T-cells or CD8(+) MOLT-3 cells were isolated at 25 μL/min through the curvilinear channel device. Following cell affinity capture, the microfluidic device was washed with 1 mL of 0.5% BSA/PBS at 55 μL/min to remove unbound cells. For the enumeration, the cells were released from the device using USER@ enzyme and collected in a glass bottom well of a 96-well plate. The cells were immunostained for identification and enumeration.

EVs were isolated from plasma or cell media using either the 3- or 7-bed EV-MAP devices. To obtain plasma and medium appropriate for EV isolation, the blood components and cell suspensions were centrifuged at 300×g for 5 min followed by 1000× g for 10 min before the plasma or medium was infused into the microfluidic chip. All samples were hydrodynamically driven through the chip using a syringe pump (New Era Pump Systems, Inc., Farmingdale, N.Y., USA) and syringe fitted with a capillary connector. To minimize non-specific adsorption, mAb-modified EV-MAP surfaces were blocked with 1% polyvinylpyrrolidone (PVP) and 0.5% BSA in PBS (200 μL, 10 μL/min), then washed with 1% Tween20 in TBS after enrichment to remove non-specifically bound material. The cell media or plasma samples were infused into the 3-bed EV-MAP at 5 μL/min. Post-isolation rinse was performed at 10 μL/min with TBS/Tween20 (Bio-Rad, Hercules, Calif.). All the buffer solutions used for rinsing were filtered using a 0.45-μm polypropylene housing, surfactant-free cellulose acetate membrane filter (Thermo Scientific) prior to use.

Fluorescence visualization of EVs membrane antigens. Following EV isolation, devices were incubated with APC conjugated mouse anti-human CD8α mAb (R&D systems/BioTechne) for 40 min. As controls, the same procedure was carried out for an UV/03-modified device without anti-CD8α mAb, but following EV enrichment. Isotype control experiments were performed by incubating a micro-fluidic device with APC-conjugated mouse anti-CD8α mAb. The devices were washed with TBS/Tween20 to remove any excess dye-labeled mAb and washed with PBS prior to fluorescence imaging. The devices were visualized using a 200M inverted microscope (Zeiss) with a 20× objective (0.3 NA, Plan NeoFluar), XBO 75 Xe arc lamp, single band Cy5 filter set (Omega Optical), Cascade: 1K EM-CCD camera (Photometric), and MAC 5000 stage (Ludl Electronic Products), all of which were computer-controlled via Micro-Manager. The final images were background subtracted and analyzed using Image-J software.

Transmission electron microscopy. Enriched and subsequently released EVs in ˜150 μL of PBS/USER@ were vortexed thoroughly and 5 μL of the EV samples were placed onto a grid carbon (Carbon Type-B, 300 mesh, Copper, TED PELLA, Inc., Redding, Calif.) film side for 20 min. Then, the grid was washed with deionized water. Next, the grid was placed for 10 s in 2% (w/v) uranyl acetate stain filtered with a 0.22-μm filter (Thermo Scientific, IL, USA), and blot dried. The grids were dried for at least 15 min before viewing through the microscope (FEI TECNAI F20 XT field emission transmission electron Microscope, 200 kV electron source—Schottky field emitter).

Nanoparticle tracking analysis. EVs enriched and subsequently released in ˜150 μL of PBS/USER@ from the microfluidic device were analyzed via NTA (Nanosight NT 2.3). The samples were diluted 100×, and just before analysis they were vortexed thoroughly. The instrument parameters used for the analysis were: Camera shutter 1206, camera gain 366, capture duration 90 s. Five videos were taken for each sample at a temperature of 25° C. The flow cell was washed five times with PBS in between sample analysis. During the final wash with PBS, the video was monitored to check if there were any particles left in the flow cell. If particles were detected in the video, washing was continued until no particles were seen. NTA was also used to evaluate the EV release efficiency by performing two consecutive rounds of USER@ enzyme release and quantification of particles following each round.

Sample processing in stroke model experiments. To assess the changes in mRNA transcript abundance following an inflammation event, the experiments were performed with LPS naïve and LPS-stimulated cells grown in FBS depleted of EVs. Cell cultures with ˜1×106 cells/mL were stimulated with LPS. After 24 h, the cell suspension was centrifuged for 10 min at 300× g to pellet the cells. The cell pellet was resuspended in 5 mL of PBS, and 1 mL of the suspension was infused at 25 μL/min through the curvilinear channel device. Following cell affinity capture, the microfluidic device was washed with 1 mL of 0.5% BSA/PBS at 55 μL/min to remove unbound cells. EVs processing was as follows: after centrifugation of the cell media, the supernatant was used as source of EVs. The same isolation protocol was used as in “T-cell and EV affinity purification” section.

Following affinity selection, total RNA (TRNA) was isolated from cells and EVs, reverse transcribed, and subjected to mRNA expression analysis. ddPCR provided absolute quantification of the target cDNA (i.e., mRNA).

TRNA extraction from cells and EVs followed the same protocol; lysis was performed with lysis buffer provided in Zymo RNA kit. The lysate was introduced into a purification column. Further steps were completed according to the manufacturer's protocol. Purified TRNA was eluted in ˜8 μL of water. The profiles of extracted TRNA were analyzed and quantified using gel electrophoresis—an Agilent 2200 TapeStation using 2 μL of eluent.

cDNA synthesis from purified RNA. Purified RNA isolated from cells or EVs was eluted from the purification column in ˜8 μL of water. Two microliters of the eluent was taken for RNA quantification, and the remaining solution was used for the complementary DNA (cDNA) synthesis (2 μL for each RT(+) and RT(−) reactions in 20 μL total volume). cDNA was synthesized via reverse transcription (RT) reaction with poly-dT primer using ProtoScript II First Strand cDNA Synthesis Kit according to the manufacturer's instructions. Negative RT control reactions were performed in the absence of the enzyme.

Droplet digital PCR. Synthesized cDNA was used in ddPCR for gene expression analysis. The procedure for the PCR reaction preparation with 2 μL of the cDNA in 20 μL total reaction volume and 0.125 μM concentration of the primers followed manufacturer's suggestions. EVA Green Supermix (BioRad) was used for the PCR mix preparation. The primers for the genes vFOS (FBJ murine osteosarcoma viral oncogene), VCAN (Versican), PLBD1 (phospholipase B domain containing 1), MMP9 (metallopeptidase 9), and CA4 (carbonic anhydrase 4) were purchased from Integrated DNA Technologies. The primer sequences are given in Table 13. Droplet formation of the PCR mix in oil was performed with QX200 Droplet Generator according to manufacturer's protocol. PCR reactions were carried out in a C1000 touch thermal cycler (BioRad) with the following steps: 95° C. for 5 min; 40 cycles of denaturation at 95° C. for 30 s; annealing at 52° C. for 30 s; and extension at 72° C. for 1 min. A final cooling step was carried out at 4° C. To read the droplets, a BioRad QX-200 ddPCR system was used, with the data analyzed using the QuantaSoft™ software. ddPCR results were normalized to ng of TRNA.

Clinical samples. Blood samples from healthy donors and plasma samples for the AIS patients were obtained from the Biorepository at the University of Kansas Medical Center, Kansas City, Kans., and SUNY Down State Medical Center, New York, N.Y., respectively. Dr. Alison Baird lab personnel at SUNY Downstate Medical Center collected the clinical plasma samples according to this institution's IRB protocol. Informed consent was obtained from all patients. Plasma samples were stored at −80° C. until analysis.

Analysis of clinical samples. Ten single-blinded clinical samples, out of which five were AIS patient samples and five were healthy controls, were analyzed. The devices were pretreated in the same way as the 3-bed device. Following EV enrichment and wash, the selected EVs were either lysed on chip and TRNA extracted or released for NTA and TEM analysis (see protocols listed above). The yields of the TRNA isolated from clinical samples are presented in Table 15.

Statistics and reproducibility. Statistical analysis using R Studio software was performed to identify patient samples. Heat maps were generated and PCA was one for the ten clinical samples and additional datasets from six healthy donor plasma.

### Results

### EV Microfluidic Affinity Purification.

Two types of devices containing microfluidic beds populated with pillars were used: (i) A 3-bed chip (FIG. 5A-5C); and (ii) a device with a z-type configuration that addressed 7 beds in parallel (FIG. 5D, 5E). Both devices were made from a thermoplastic to allow for high-rate production at low cost to accommodate diagnostic applications.

The mold master for the 3-bed EV-MAP was fabricated in brass using high-precision micromilling, and devices were replicated in cyclic olefin copolymer (COC) via hot embossing²¹. The device contained ˜15,000 circular pillars (100 μm diameter spaced ˜15 μm apart, surface area of 6.8 cm²) with a theoretical EV load capacity of 3.5×10¹⁰ particles calculated based on the surface area of the device and a monolayer hexagonal packing of EVs with a diameter of 150 nm (Table 9). With its smaller surface area, the amount of mAb required for surface loading was reduced compared to the z-chip resulting in lower assay cost and making it attractive for initial assay characterization. This chip was made from COC.

TABLE 9
Comparison of device parameters of 3-bed and 7-bed EV-MAP.

| Metric | 3-bed EV-MAP | 7-bed EV-MAP |
|---|---|---|
| Bed dimensions (l × w × d) (mm × mm × μm) | 122 × 1.7 × 90 | 23 × 3.6 × 50 |
| Number of pillars | 15,202 | 1,475,712 |
| Pillar geometry and dimensions (μm) | Circular 100 | Square 10 × 10 |
| Inter-pillar spacing (μm) | 15 | 10 |
| Internal surface area (cm²) | 6.8 | 38.5 |
| Internal volume (μL) | 6.5 | 22.4 |
| Mass of antibody (μg) immobilized on the surface | 3.9 ± 1.3 | 23.1 ± 3.4 |
| Antibody coverage (pmole/surface area) | 26.7 ± 8.7 (−3.9 pmole/cm²) | 154.0 ± 22.7 (−4.0 pmole/cm²) |
| Bed capacity for EV, d = 150 nm | 3.5 × 10¹⁰ particles | 2.2 × 10¹¹ particles |

Each z-type device contained 7 beds connected in parallel with perpendicular inlet and outlet channels arranged in a z-configuration. The device contained ˜1.5 million diamond-shaped pillars (10 μm×10 μm, 10 μm spacing) providing a ˜38.6 cm² surface area with a maximum theoretical particle load of 2.2×10¹¹ (Table 9). This chip was made from cyclic olefin polymer (COP).

Measurements of polymer surface hydrophilicity/hydrophobicity via water contact angle and carboxylic acid group densities via a TBO assay following activation²² showed no significant differences between the two thermoplastics providing similar surface densities of mAbs. Covalently attached anti-CD8α antibody was removed from the chip and its concentration was evaluated using a BCA assay, which concluded that for both COC and COP the mAb surface density was ˜4 pmole/cm² (Table 10).

TABLE 10
Empirical data for the protein content evaluated following affinity isolation of EVs using the 3-bed EV-MAP.
Volumetric flow rate at which 100 μL of healthy plasma processed

| | 0.5 μL/min | 1.0 μL/min | 2.0 μL/min | 5.0 μL/min | 10.0 μL/min |
|---|---|---|---|---|---|
| Protein mass (μg) | 12.7 ± 3.4 | 24.8 ± 4.0 | 13.1 ± 4.1 | 8.5 ± 4.3 | 7.5 ± 4.06 |

Volume of healthy plasma processed on a 3-bed EVMAP at 10 μL/min

| | 0 μL | 100 μL | 300 μL | 500 μL | 1000 μL |
|---|---|---|---|---|---|
| Protein mass (μg)-BSA modified chip | 3.9 | 5.6 ± 2.1 | 13.7 ± 3.1 | 13.2 ± 2.1 | 12.8 ± 2.9 |
| Protein mass (μg)-anti-CD8 Ab modified chip | 3.3 | 7.5 ± 4.1 | 22.7 ± 3.0 | 96.1 ± 6.1 | 106.2 ± 7.1 |

The architecture of the EV-MAP was designed to maximize EV recovery while providing high throughput processing to keep the processing time short. In the design, sample infused into the device moves around solid pillars with EVs diffusing laterally for potential association with mAbs immobilized onto the micro-pillar surfaces. Small inter-pillar spacing and long bed lengths decreased the diffusional distances and provided sufficient residence time, respectively, for increasing EV binding probability to the mAbs. To guide the design, we developed a Monte Carlo simulation that incorporated hydrodynamic Poiseuille flow, lateral and longitudinal EV diffusion, and EV-mAb binding kinetics per the Chang-Hammer model²³. In the model, EVs were tracked until they were either bound to the surface or lost and the results were used to determine the recovery (FIG. 5E). The 3-bed EV-MAP has an inter-pillar spacing of ˜15 μm and a bed length of 122 mm with large micropillars (d=100 μm) that resulted in a recovery of 41% at 5 μL/min (FIG. 5F). The 7-bed EV-MAP pillar size and inter-pillar spacing was 10 μm and yielded a 97% EV recovery at 5 μL/min (FIG. 5F). The 7-bed EV-MAP provided high sample processing capabilities in comparison with the 3-bed device. Assuming the same recovery, the 7-bed device can operate at an 8-fold higher throughput (FIG. 5G). In the pilot study presented herein, clinical samples collected from AIS patients were processed using the 7-bed EV-MAP to ensure performing ddPCR with a sufficient dynamic range and above the limit-of-detection for mRNA quantification while, at the same time, keeping the sample processing time short. The 3-bed EV-MAP was used for initial assay characterization prior to clinical testing.

Modeling experiments were verified with empirical data evaluating EV protein concentration from enriched CD8α(+) EVs from healthy donor plasma as a function of the volumetric flow rate (Table 10). A similar trend was observed in both the COMSOL simulation and experimental data with higher recoveries (based on protein content) achieved at lower volumetric flow rates. The recovery of EVs determined using a “self-referencing” method²⁴ was 48% for sample processed at 5 μL/min, which agreed favorably with the simulation predicted recovery of 41% at this same volumetric flow rate. The saturation of the 3-bed EV-MAP was observed for ˜500 μL of healthy donor plasma processed on chip for the affinity selection of CD8α(+) EVs. The amount of protein extracted was 96 μg (Table 10). To determine the amount of protein non-specific adsorption, we modified the surface of the EV-MAP devices with bovine serum albumin (BSA) instead of anti-CD8 mAb and processed the same volume of healthy donor plasma (Table 9). The amount of protein non-specifically bound to the bed was ˜10× lower, ˜9 μg for 500 μL of plasma sample. Four micrograms of protein was attributed to BSA, while the remaining 5 μg of protein mass could either be from EVs or plasma proteins.

### Affinity Enrichment of EVs Using EV-MAP.

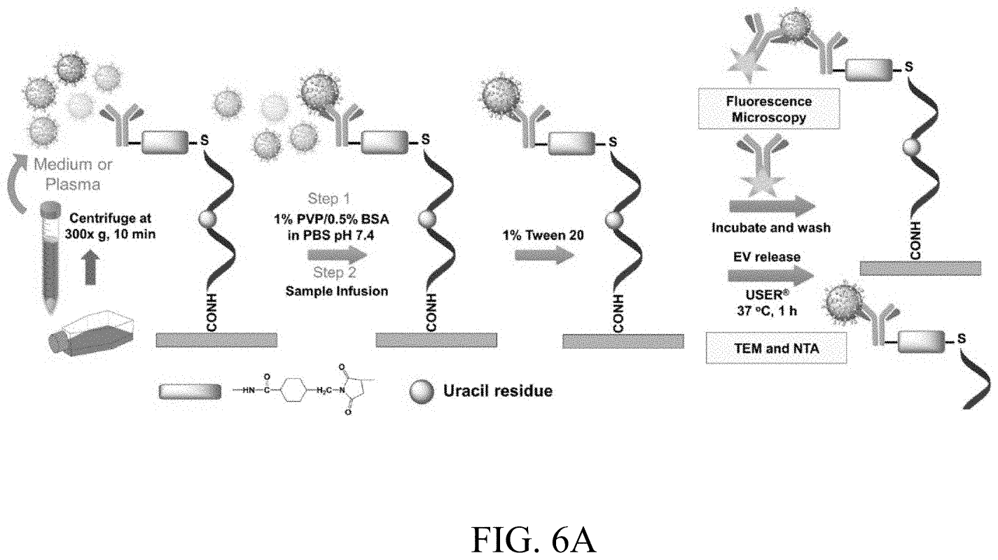

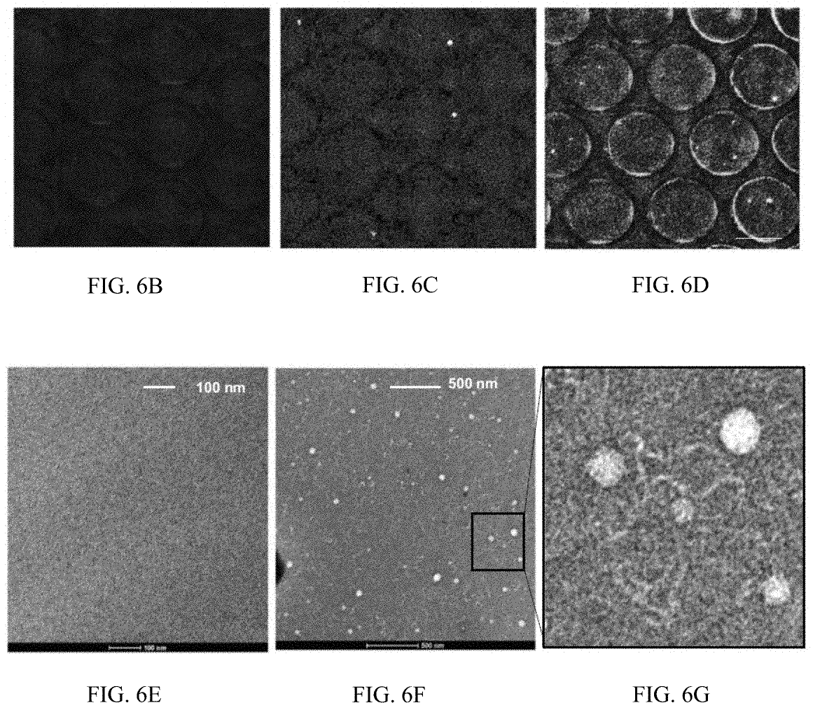

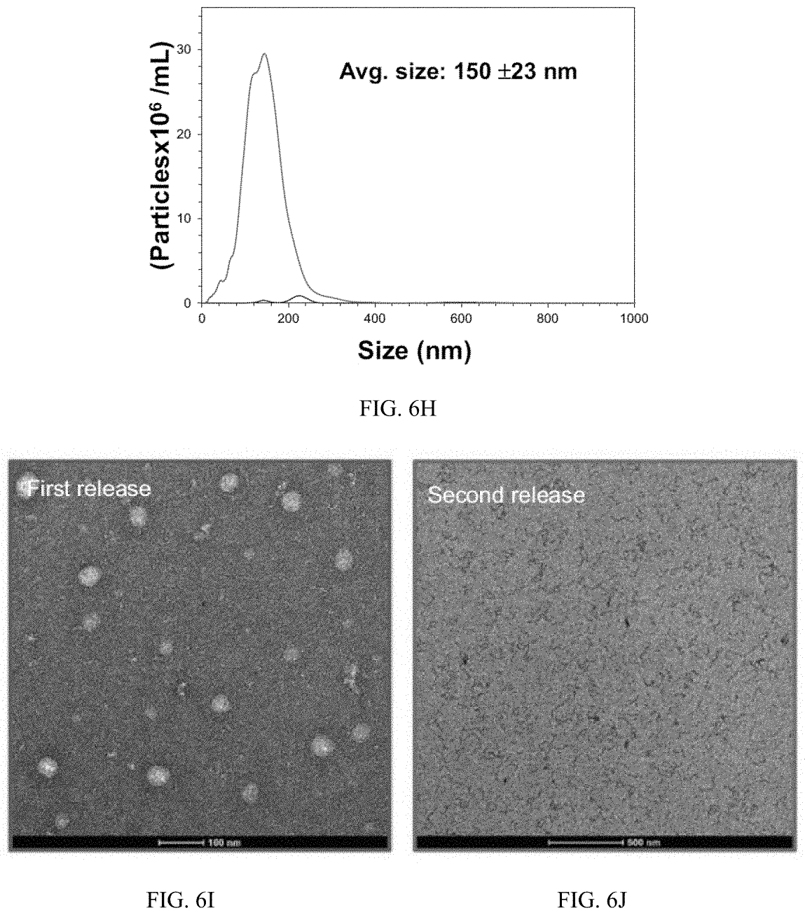

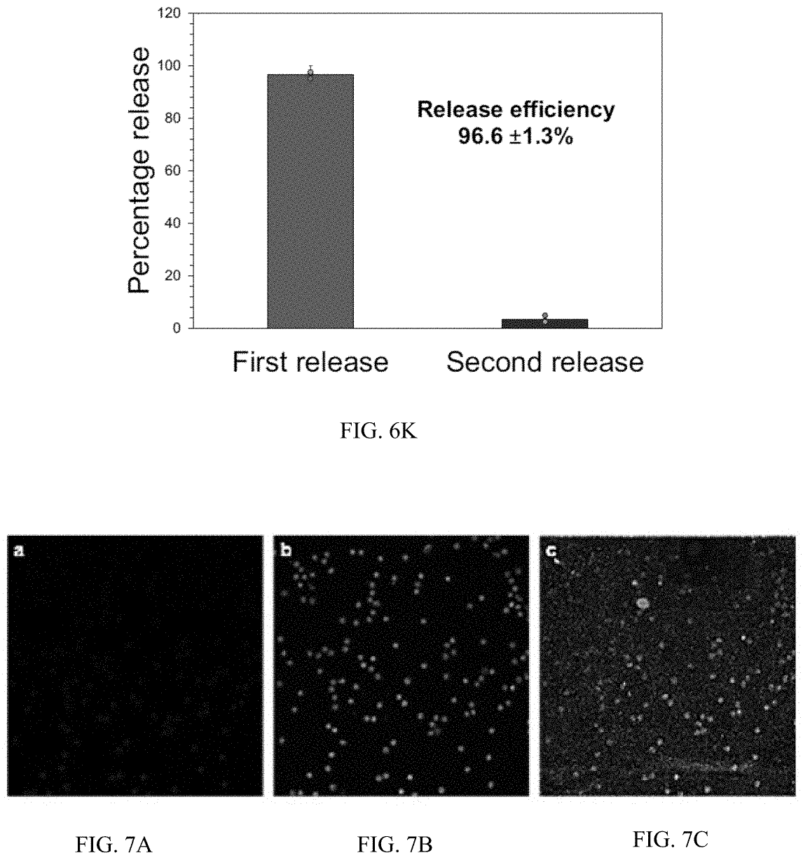

Anti-CD8α mAbs were covalently attached to the surfaces of the EV-MAP with an oligonucleotide bifunctional linker (FIG. 6A)²⁴, which contained a uracil residue that can be cleaved with USER@ (Uracil Specific Excision Reagent) hence allowing for release of EVs after affinity enrichment, similar to what we have shown for affinity-enriched circulating tumor cells²⁴.

We evaluated the EV-MAP performance metrics by isolating and releasing EVs originating from the MOLT-3 cell line (T-cell model) cultured in EV-depleted fetal bovine serum (FBS). MOLT-3 cells showed CD8 expression at an average of 2500 CD8α receptors/cell (FIG. 7) in 13% of the population, similar to that seen for this same cell line in previous reports²⁵.

Following enrichment of EVs, we used an immunoassay with APC-labeled anti-CD8α mAbs to target surface EV-CD8α antigens. Imaging showed higher fluorescence intensity when devices were modified with anti-CD8α mAbs compared to control devices, which contained no mAbs or an isotype (FIG. 6B-6D).

The mAb linker cleaved with USER@ released enriched EVs from the EV-MAP as confirmed by TEM and nanoparticle tracking analysis (NTA). TEM indicated the presence of EVs (FIG. 6E-6G) in the device's eluent following release with NTA indicating an average particle size of 150±23 nm and a concentration of 1.6±0.7×10⁸ particles/100 μL media (n=3), suggesting we are operating below the theoretical EV load capacity of the 3-bed chip. Incubation with USER@ yielded a 96.6±1.3% EV release efficiency (FIG. 6H-6K).

EV mRNA Expression in an Inflammation Model.

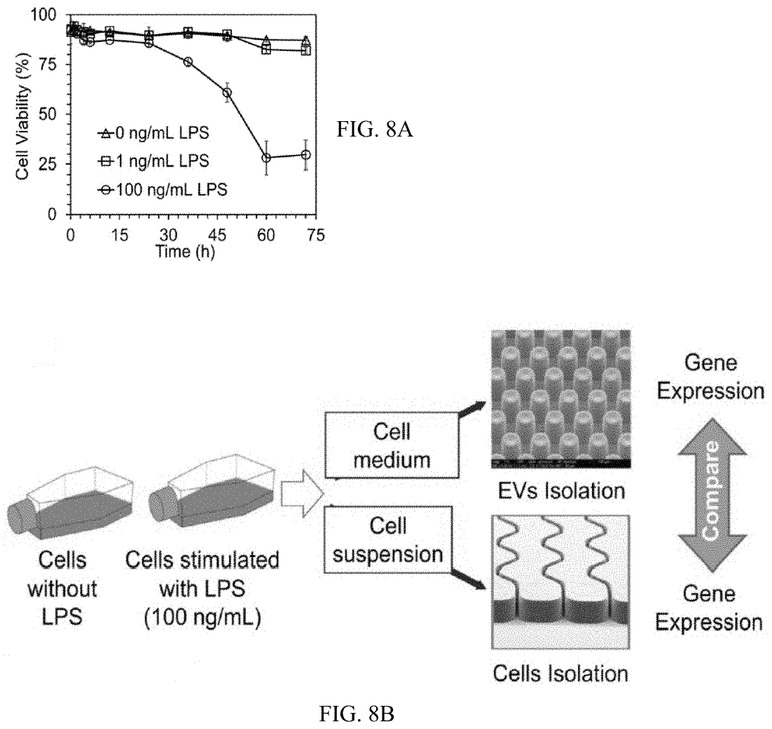

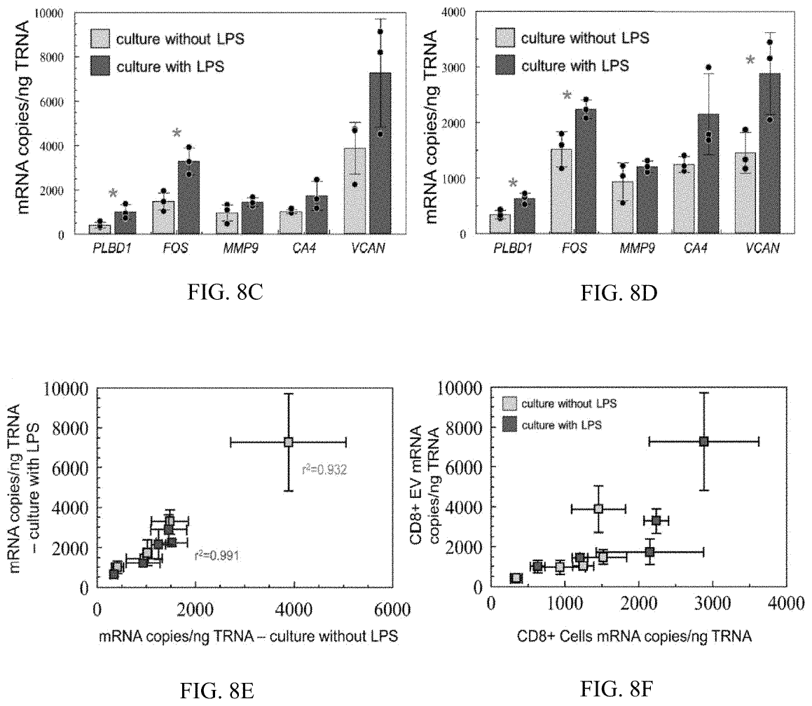

By evaluating the response of MOLT-3 cells and the EVs they generate when exposed to lipopolysaccharide (LPS), we mimicked the inflammation process during an AIS event²⁶. LPS is a component of the outer membrane of Gram-negative bacteria that induces cell inflammation in macrophages and T-cells by releasing cytokines²⁷. LPS stimulation conditions in cell culture (FIG. 8A) were optimized to eliminate potential DNA or RNA damage by reactive oxygen species generated in response to LPS²⁸. The MOLT-3 cell line was used as the model because of its expression of CD8 antigens. CD8 expressing T-cells have been found to possess mRNA expression profiles indicative of AIS⁷.

MOLT-3 cell viability was evaluated over a 72-h period cultured with 0, 1, and 100 ng/mL of LPS. Cells showed 80±2% viability and no obvious changes in cell morphology after 24 h of stimulation with 100 ng/mL LPS (FIG. 8A). Therefore, these conditions were applied to the cell culture. Cells were processed through an anti-CD8 mAb modified sinusoidal microfluidic device (FIG. 8B)²². EVs in the conditioned media were affinity-enriched with the 3-bed EV-MAP also modified with anti-CD8α mAbs (FIG. 8B).

We selected a gene cluster comprising a gene set from Example 1, which consisted of five genes: FOS, VCAN, PLBD1, MMP9, and CA4. These genes were selected because they have already shown statistical significance in mRNA expression between AIS patients and controls prior to their identification as a cluster. FOS and PLBD1 have both shown that they have statistical significance in mRNA expression when considered alone, (p values of 0.043 and 0.0034 respectively) (See Table 2) and when considered as part of a cluster (cluster 1, p value of 1.01×10⁻⁹) (See Table 4). Furthermore, VCAN has also shown statistical significance with FOS and PLBD1 as part of cluster 1 (p value of 1.01×10⁻⁹) and with other genes (cluster 10, p value of 0.002) (See Table 4). MMP9 (cluster 2, p value of 1.50×⁻⁶) and CA4 (cluster 3, p value of 1.52×10⁻⁵ and cluster 9, p value of 8.2×10⁻⁸) has shown statistical significance as part of a gene cluster. (See table 4) In certain embodiments, other gene clusters may be used. Clusters of interest include for example those clusters identified in Table 4, Table 11, and Table 12.

TABLE 11
Gene expression clusters significantly characteristic for IS identified in
hierarchical cluster analyses in 4 leukocyte subsets

| | P value, of | | |
|---|---|---|---|
| | cluster, stroke | Adjusted | Adjusted p |
| Transcripts | versus control | p value* | value** |
| CD15-Cluster 1 | | | |
| IQGAP1, SLC16A6, NPL, CD93, PYGL, PLBD1 | 9.4e−6 | 8.84e−5 | 4.41e−4 |
| CD15-Cluster 2 | | | |
| ADM, CKAP4, FOS, BST1 | 2.94e−5 | 1.73e−4 | 1.38e−3 |
| CD15-Cluster 3 | | | |
| ENTPD1, IL13RA1, LTA4H, S100P | 9.7e−5 | 3.80e−4 | 4.56e−3 |
| CD15-Cluster 5 | | | |
| DUST1, HIST2H2AA3, BCL6, PILRA, FCGR1A, TLR2 | 7.70e−5 | 3.29e−4 | 3.62e−3 |
| CD15-Cluster 7 | | | |
| LY96, S100A9, FPR1, S100A12, RNASE2, CCR7 | 0.0012 | 3.01e−3 | 5.73e−3 |
| CD15-Cluster 8 | | | |
| CA4, MMP9, NAIP | 6.14e−7 | 9.62e−6 | 2.88e−5 |
| CD14-Cluster 4 | | | |
| PLBD1, BST1, LTA4H, CYBB, SCL16, BCL6, | 0.00019 | 6.38e−4 | 8.93e−3 |
| VCAN, FCGR1A | | | |
| CD4-Cluster 3 | | | |
| IQGAP1, NPL, FOS, PLBD1, BST1, VCAN | 0.000146 | 5.28e−4 | 6.86e−3 |
| CD8-Cluster 1 | | | |
| IL13, APLP2, ENTPD1, ETS2, PYGL, DUSP1, | 3.64e−7 | 8.55e−6 | 1.71e−5 |
| KIAA, ADM, S100P, CD36, | | | |
| CD8-Cluster 3 | | | |
| CYBB, BST1, CD93, NPL, IQGAP1 | 0.00021 | 6.58e−4 | 9.87e−3 |
| CD8-Cluster 4 | | | |
| FOS, VCAN, PLBD1, MMP9, CA4 | 1.42e−5 | 1.11e−4 | 6.67e−4 |
| CD8-Cluster 5 | | | |
| BCL6, SLC16, LTA4H, CKAP4, FPR1, FCGR1A | 2.58e−5 | 1.73e−4 | 1.21e−3 |
| γδT-Cluster 1 | | | |
| IQGAP1, NPL, FOS, DUSP1, CD93, CKAP4, | 7.52e−6 | 8.84e−5 | 3.53e−4 |
| PLBD1, BST1, VCAN | | | |
| γδT-Cluster 4 | | | |
| ETS2, IL13, ENTPD1, PYGL, ADM, KIAA, | 5.19e−5 | 2.44e−4 | 2.44e−3 |
| APLP2, MMP9, CA4 | | | |

Wilcoxon rank sum tests used for analyses,
*-FDR,
**-Bonferroni;
CD15-granulocytes;
CD14-monocytes;
CD4, CD8, γδT-respectively CD4⁺, CD8⁺, γδTCR T lymphocytes

TABLE 12
Patterns of Altered Gene Expression

| Pattern | Transcripts |
|---|---|
| Elevated in CD15 cells | NAIP |
| | MMP9 |
| | ADM |
| | LTA4H |
| | PYGL |
| | FCGR1A |
| | IL13RA1 |
| Elevated in both CD15 and | FOS |
| CD8 cells | LY96 |
| | S100A9 |
| | S100A12 |
| | CA4 |
| | ETS2 |
| Elevated in CD15 cells | S100P |
| and/or >1 T cell subset (CD8 | DUSP1 |
| and γδT) | |
| Elevated in CD8 cells | ENTPD1 |
| | PILRA |
| | PLBD1 |
| | F5 |
| | NPL |
| | FPR1 |
| Elevated in CD8 and CD4 | VCAN |
| (decreased in CD15) cells | |

This gene panel evaluated in CD8(+) T-cells has shown statistically significant differences (p=1.42×10⁻⁵) in mRNA expression between AIS patients and controls⁷. We used ddPCR (see Table 13 for primer sequences that were designed to be close to the 3′ end of the mRNA) to provide absolute quantification of cDNA from the target mRNA.

TABLE 13
Primer Sequences used in gene expression analysis

| | Primer F | Primer R | Amplicon | Span from |
|---|---|---|---|---|
| Gene | 5′-3′ (Tm/° C.) | 5′-3′ (Tm/° C.) | size (bp) | polyA (nt) |
| vFOS | TGCCAGGAACACAGT | TTCAGAGAGCTGGTA | 188 | 301 |
| cDNA clone | AG (51.4) | GTTAG (50.7) | | |
| MGC: 11074 | (SEQ ID NO: 81) | (SEQ ID NO: 86) | | |
| IMAGE: 3688670 | | | | |
| VCAN | TCTCAAAGAAACAGA | AGAGCCACAGAGCAT | 156 | 390 |
| cDNA clone | GTGATA (49.9) | TT (51.1) | | |
| IMAGE: 5218077 | (SEQ ID NO: 82) | (SEQ ID NO: 87) | | |
| PLBD1 | GTACTGAGATGCTAG | CAAGGGAAAGTGACT | 189 | 470 |
| NM_024829.5 | GTAGATA (50.2) | GATAC (50.4) | | |
| | (SEQ ID NO: 83) | (SEQ ID NO: 88) | | |
| MMP-9 | GGGATTTACATGGCA | ACCGAGAGAAAGCCT | 162 | 370 |
| NM_004994.2 | CTG (50.8) | ATT (50.2) | | |
| | (SEQ ID NO: 84) | (SEQ ID NO: 89) | | |
| CA4 | GAAGCCTGGAACTTG | AGCGCACGGTGATAA | 164 | 240 |
| cDNA clone | GA (51.7) | A (51.4) | | |
| MGC: 71638 | (SEQ ID NO: 85) | (SEQ ID NO: 90) | | |
| IMAGE: 30331755 | | | | |

cDNA copies were normalized to ng TRNA quantified by gel electrophoresis (Table 14 for typical ddPCR results).

TABLE 14
Representative data from droplet digital PCR (Clinical sample and MOLT-3 cell line)

| | | | | | Sample data from MOLT-3 cell line | | | |
|---|---|---|---|---|---|---|---|---|
| | Sample data from a clinical sample | | | | sample | | | |
| | Copies | | Negative | | Copies | | Negative | |
| | per | Accepted | Copies | Accepted | per | Accepted | Copies | Accepted |
| Gene | 20 μL | Droplets | per 20 μL | Droplets | 20 μL | Droplets | per 20 μL | Droplets |
| PLBD1 | 36 | 14725 | 7.6 | 12297 | 94 | 15637 | 0 | 15717 |
| vFOS | 130 | 14612 | 4 | 12043 | 272 | 16272 | 4.4 | 15716 |
| MMP9 | 112 | 15310 | 9 | 12949 | 168 | 17055 | 0 | 16976 |
| CA4 | 104 | 14774 | 4 | 12043 | 248 | 15866 | 1.4 | 15992 |
| VCAN | 340 | 14877 | 13 | 12707 | 456 | 14291 | 5.8 | 16114 |

Among the five genes profiled, CD8(+) EVs harvested from stimulated cells showed two genes (PLBD1 and FOS) that were upregulated upon LPS stimulation (FIG. 8C). In CD8(+) MOLT-3 cells, three genes (PLBD1, FOS, and VCAN) were upregulated compared to the non-LPS stimulated cells. The mRNA copy numbers in cells and EVs isolated from the medium with LPS were on average twice as abundant as unstimulated cells (slopes of 1.7 and 1.9 for MOLT-3 cells and EVs, respectively, FIG. 8E). We observed a 1:1 transcript ratio for PLBD1, FOS, MMP9, and CA4 in CD8(+) EVs and CD8(+) MOLT-3 cells in both stimulated and unstimulated conditions (0.84-1.02 slopes, FIG. 8F). VCAN appeared to be 2.5× more abundant in CD8(+) EVs than in CD8(+) MOLT-3 cells in both stimulated and unstimulated conditions (FIG. 8F). Although we could differentiate stimulated and unstimulated cells using three genes based on these model experiments, it is important to note that in clinical studies, a five-gene panel will be used, as gene expression data for leukocyte subpopulations from clinical samples have identified the five-gene panel providing the highest clinical specificity and sensitivity for AIS⁷.

Gene Expression of CD8(+) T-Cells and CD8(+) EVs Isolated from Healthy Donors' Plasma.

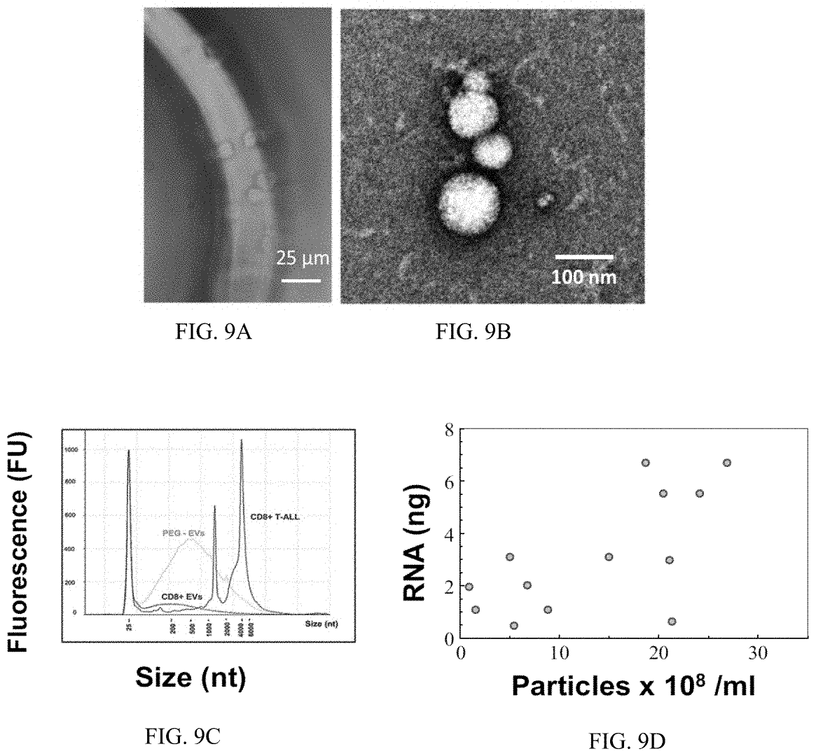

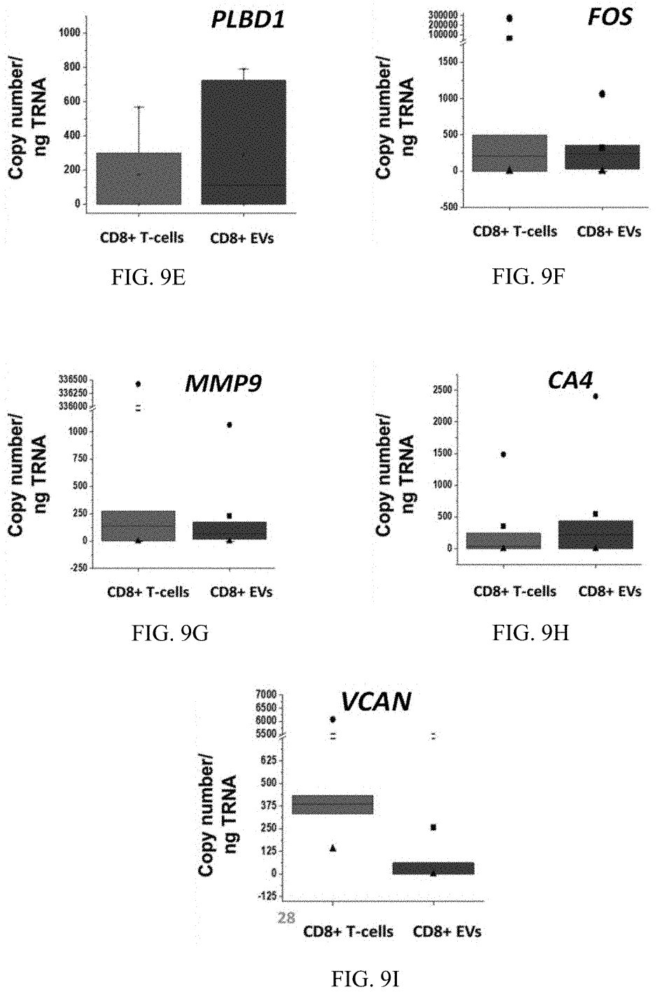

We isolated CD8(+) T-cells from healthy donor blood samples using a sinusoidal microfluidic device (FIGS. 8B and 9A)², and enriched CD8(+) EVs from plasma from the same blood samples. Following affinity enrichment and subsequent release from the capture surface, T-cells were enumerated (˜24,000±3000 cells/mL of blood) and immunostained against CD45 and CD8α antigens (FIG. 7) showing a purity of the T-cell fraction of 81.3±11.5%. EVs following release were visualized and characterized using TEM (FIG. 9B).

We isolated 8.8 ng TRNA from CD8(+) T-cells enriched from 1 mL of blood as determined by TRNA gel electrophoresis. Cells' TRNA profiles were typical for eukaryotic cells (rRNA with a 28S band twice as intense as the 18S band, FIG. 9C). EVs were isolated from 500 μL of plasma using the 3-bed EV-MAP and PEG precipitation²⁹. We extracted 3.3 ng and 18.1 ng EV-TRNA following affinity isolation and PEG precipitation, respectively. Gel electrophoresis analysis of the isolated TRNA using EV-MAP and PEG indicated the presence of short RNA fragments and a lack of 28S/18S rRNA bands in the EV-enriched fractions. The RNA size ranged between 50 and 2000 nt with the highest abundance for 200 nt long fragments (FIG. 9C) indicating the presence of truncated mRNA and rRNA fragments, miRNA, and long non-coding RNAs^{30,31}. We observed a positive correlation between the amount of TRNA extracted and the number of nanoparticles detected via NTA (Pearson coefficient=0.671) and ˜2×10-18 g TRNA/EV particle (FIG. 9D).

Analysis of the ddPCR data for six healthy donors indicated that for all genes in our AIS panel, there was no statistical difference in expression between T-cells and their generated EVs (FIG. 9E-9I). mRNA abundance for paired T-cells and their generated EVs in healthy donors show no correlation (P=−0.1892) unlike in the LPS-stimulated MOLT-3 cell line and its EVs (P=0.8244, FIG. 8F), suggesting high similarities in mRNA transcript expression between EVs and their parental cells when inflammatory conditions were applied, as is the case with LPS-stimulated cells³².

mRNA Transcripts Analysis in CD8(+) EVs Isolated from AIS Patients and Normal Controls' Plasma.

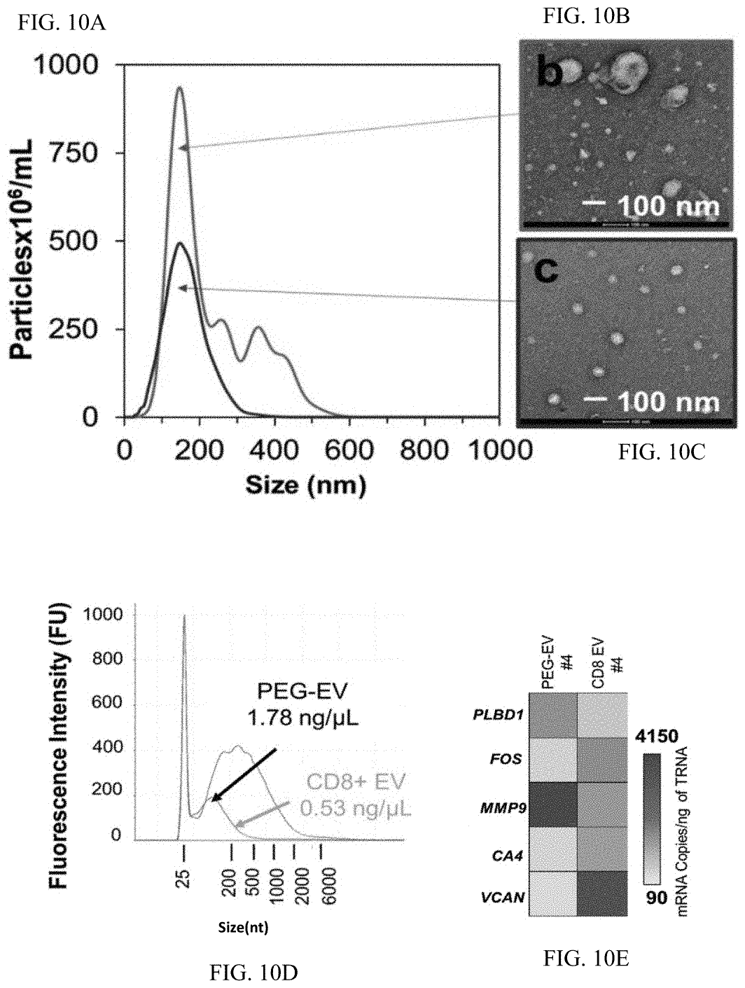

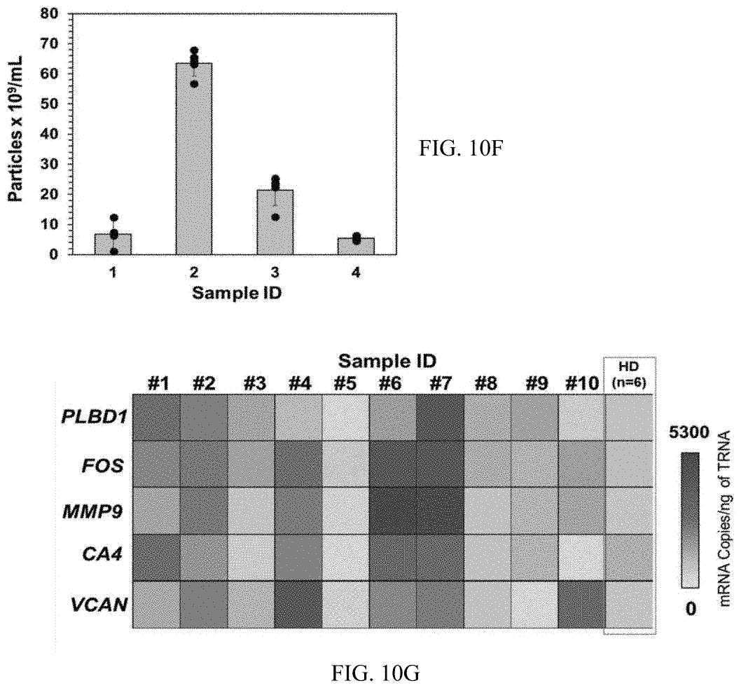

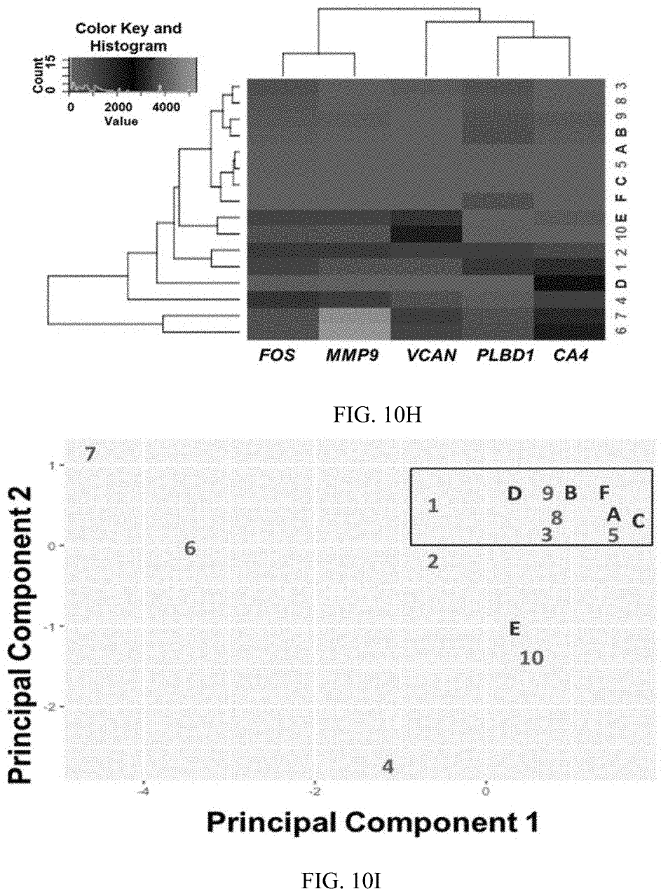

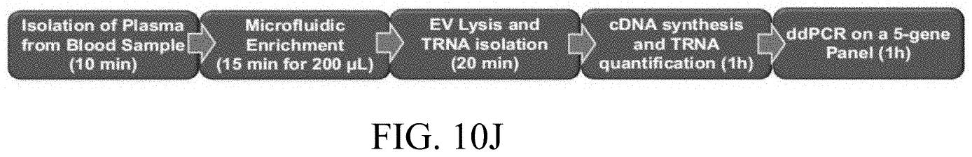

EVs from clinical samples were enriched using the 7-bed EV-MAP. For randomly selected samples, we compared EV-MAP isolation to PEG EV precipitation. NTA results showed a much narrower size distribution of particles for EV-MAP (158±10 nm) compared to PEG precipitation (230±110 nm), which agreed with EV sizes and morphology from TEM (FIG. 10B, 10C). ddPCR results for clinical samples are shown in Table 14. The yields of the TRNA isolated from clinical samples are listed in Table 15. While PEG precipitation yielded more TRNA (15.1 ng) than anti-CD8α mAb EV-MAP (4.4 ng), mRNA profiling of the isolates for our five-gene panel differed (FIG. 10E) owing to the fact that the PEG method cannot differentiate EVs by parental cell type origination because it isolates the entire EV population found in plasma.

TABLE 15
TRNA yields isolated from affinity selected EVs
from clinical samples

| Single blinded | RNA | Mass (ng) of RNA |
|---|---|---|
| patient code # | isolated (ng) | per RT (+/−) reaction |
| 1 | 1.79 | 0.48 |
| 2 | 2.16 | 0.50 |
| 3 | 1.38 | 0.35 |
| 4 | 4.44 | 1.07 |
| 5 | 2.22 | 0.50 |
| 6 | 0.54 | 0.15 |
| 7 | 0.60 | 0.14 |
| 8 | 0.44 | 0.11 |
| 9 | 0.43 | 0.11 |
| 10 | 0.64 | 0.15 |

Gene expression analysis using the five-gene AIS panel was performed for ten clinical samples and provides a proof-of-concept study to determine if mRNA sourced from EVs could be used to detect AIS. The cohort of six healthy samples analyzed previously served as a training set to differentiate between controls and AIS samples. Gene copy numbers normalized to ng TRNA (FIG. 10G) were analyzed by principal component analysis (PCA) and heat map compilation for cluster determination. In PCA, sample grouping with the training set were categorized as healthy controls (see the box in FIG. 10I), while all others were classified as AIS patients. Clustering was confirmed with heat maps (FIG. 10H, 10I). When compared with the “key”, we found 80% success in correctly identifying patient status. For both cohorts, there was no statistical difference in TRNA concentration (healthy—1.6 ng/mL average, range of 0.5-2.2 ng/mL; AIS patients—2.05 ng/mL average, range of 0.6-5.5 ng/mL). There was also no correlation between EV particle concentration and clinical condition (see FIG. 10F). The entire assay required 3.7 h from sample-to-answer and is within the therapeutic time window mandated by rt-PA treatment for AIS (4.5 h; FIG. 10J).

### Discussion

New blood-based biomarkers and the development of tests that utilize those biomarkers are needed to improve outcomes for AIS patients. mRNA transcripts packaged into peripheral white blood cells⁷ are attractive because they can reflect the physiological perturbation imposed by AIS faster (<3 h after stroke onset) than proteins (>6 h) and thus provide indications of disease within the time constraints imposed by effective rt-PA treatment.

We have shown that expression of certain genes specific to AIS in CD8(+) T-cells provided 66%, 87%, and 100% clinical sensitivity for 2.4 h, 5 h, and 24 h following stroke onset³³, suggesting that mRNA expression differences indicative of AIS have improved clinical sensitivity with time as more leukocytes respond to an inflammatory insult. We hypothesized that gene expression changes occurring in CD8(+) T-cells responding to AIS manifest themselves into similar expression changes in CD8 (+) EVs' molecular cargo. Clinical sensitivity and specificity in assays that use blood-based biomarkers are determined by the appearance of dysregulated mRNA transcripts that allow discrimination between patients and healthy controls. The molecular processing time associated with the diagnostic is another critical factor. In its current rendition, the assay we report using EVs required 3.7 h of processing time and is within the therapeutic time window of rt-PA (FIG. 10J).

In certain embodiments, the assay is performed prior to administration of rt-PA. In certain embodiments of the invention, the assay requires between about 1-3.7 hours. For example, in certain embodiments, the assay requires 1, 1.25, 1.5, 1.75, 2, 2.25, 2.5, 2.75, 3, 3.1, 3.2, 3.3, 3.4, 3.5, 3.6, or 3.7. In certain embodiments the assay requires between about 2.75 to about 3.7 hours.

The EV-MAP platform used for enriching the EVs provided high throughput (i.e., short processing time) and sufficient EV recovery to meet the limit-of-detection requirements of ddPCR. Although the EV particle number is typically high, >109 particles per 100 μL of sample, EV mRNA expression profiling is challenged by the fact that the mass of TRNA is low per EV particle (˜2 ag per particle, FIG. 9D), and mRNA represents ˜2% of the TRNA EV cargo³¹. To add to the challenges of EV mRNA expression profiling, full length transcripts in EVs with an exosomal origin are rarely found³⁴. This was evident from the fact that the TRNA found in the EV-MAP CD8α enriched fraction was predominately <200 nt in length.

In an in vitro inflammation model that can induce potential mRNA changes during AIS, we determined high concordance in gene expression between cells and EVs (FIG. 7E) under inflammation induced with LPS (FIG. 7F)³². In clinical samples, 4.25±2.1×10⁹ CD8 expressing particles/100 μL were isolated from AIS plasma with an average particle size of 158±10 nm suggesting the presence of exosomes and/or microvesicles. The particle number did not vary considerably between healthy and AIS patients indicating EV NTA results alone are not sufficient for AIS diagnostics. The EV-mRNA assay provided 80% test positivity, similar to test positivity using MRI (83%)³⁵ and vastly better than test positivity using CT (26%). To improve the clinical sensitivity, larger gene panels (such as those in Table 4) can be used⁷ along with enrichment of both CD8(+) and CD15(+) EVs; CD15 (+) neutrophils have been identified as a source of mRNA markers for AIS as well⁷.

While PEG precipitation of EVs provided a slightly higher mass yield of TRNA compared to EV-MAP (FIG. 9C), the challenge is that the EV-enriched fraction originates from all EV subpopulations and not only from the CD8+ fraction. As a consequence, mRNA expression profiles specific to the disease are obscured by mRNA from “non-diseased” EVs; FIG. 10E showed different gene expression profiles for sample #4 when using EVs enriched from the EV-MAP versus PEG. Only when EVs were secured using affinity isolation by EV-MAP was this sample correctly identified as an AIS patient.

We successfully developed a microfluidic with the ability to affinity-enrich and release EVs for NTA, TEM, and ddPCR analyses. The 7-bed EV-MAP, compared to the 3-bed device, increased the dynamic range for mRNA expression analysis and avoided bed saturation that may bias mRNA expression results. Owing to the beds' parallel configuration compared to the serial arrangement associated with the 3-bed device, input volumes of sample can be processed in a shorter time. For example, EV recovery of 70% was achieved at 20 μL/min volumetric flow using the 7-bed EV-MAP (FIG. 5G), while the 3-bed device's recovery at this volumetric flow rate was considerably poorer.

An advantage of using EVs compared to biological cells for mRNA profiling may be their higher abundance, supplying larger amounts of mRNA responding to an inflammatory event indicative of AIS in a shorter period of time. This has been shown in early stage cancer diagnosis³⁶. To verify the time-dependent evolution of mRNA profiles following AIS onset in EVs, an in vitro AIS animal model can be used³⁷.

Several processing steps can be altered in the reported assay to reduce the processing time as well as increase the level of assay automation. The EV release process can be shortened using a coumarin-based photocleavable linker, enabling high efficiency release of EVs within 2 min without damaging the mRNA cargo³⁹; 60 min was required for USER@ enzyme cleavage of the uracil-containing oligonucleotide bifunctional linker²⁴. The TRNA normalization strategy, which utilized gel electrophoresis (see FIG. 10D) for ddPCR, can be converted to a “per particle” normalization by utilizing an on-line chip-based nanopore counting strategy of EVs. We are also designing an integrated fluidic processor consisting of the EV-MAP, an in-plane nano-pore sensor, EV lysis with TRNA solid-phase extraction unit, a continuous flow reverse transcription reactor, and a nanosensor for the label-free quantification of specific mRNAs using a solid-phase ligase detection reaction. This will eliminate the need for specialized operators to carry out the molecular test through assay automation to enable mRNA expression profiling even at the point-of-care, which will provide more timely results to allow more AIS patients to receive rt-PA treatment.

This assay and the associated hardware is intended to operate as a point-of-care test that can be performed by first responders and the results of which could be secured even before a potential patient reaches the hospital. Ideally, if the patient tested positive for AIS, he/she would be transported to a stroke center that offers the critical elements for more timely decisions on the administration of rt-PA therapy.

### REFERENCES FOR EXAMPLE 3

1. Dreyer, R. et al. Most important outcomes research papers on stroke and transient ischemic attack. Circ. Cardiovasc. Qual. Outcomes 7, 191-204 (2014).
2. Jauch, E. C. et al. Guidelines for the early management of patients with acute ischemic stroke: a guideline for healthcare professionals from the American Heart Association/American Stroke Association. Stroke 44, 870-947 (2013).
3. Hill, M. D. What Kind of Stroke Is It? Clinical Chemistry 54, 1943-1944 (2008). https://doi.org/10.1373/clinchem.2008.117382.
4. Fonarow, G. C. et al. Door-to-needle times for tissue plasminogen activator administration and clinical outcomes in acute ischemic stroke before and after a quality improvement initiative. JAMA 311, 1632-1640 (2014).
5. Kalafut, M. A., Schriger, D. L., Saver, J. L. & Starkman, S. Detection of early CT signs of >1/3 middle cerebral artery infarctions: interrater reliability and sensitivity of CT interpretation by physicians involved in acute stroke care. Stroke 31, 1667-1671 (2000).
6. Yilmaz, G., Arumugam, T. V., Stokes, K. Y. & Granger, D. N. Role of T lymphocytes and interferon-in ischemic stroke. Circulation 113, 2105-2112 (2006).
7. Adamski, M. G. et al. CD15+ granulocyte and CD8+T lymphocyte based gene expression clusters for ischemic stroke detection. Med. Res. Arch. 5, 1-13 (2017).
8. Yoon, C. et al. Premorbid warfarin use and lower D-dimer levels are associated with a spontaneous early improvement in an atrial fibrillation-related stroke. J. Thromb. Haemost. 10, 2394-2396 (2012).
9. Rothermundt, M., Peters, M., Prehn, J. H. & Arolt, V. S100B in brain damage and neurodegeneration. Microsc. Res. Tech. 60, 614-632 (2003).
10. Ji, Q. et al. Increased brain-specific MiR-9 and MiR-124 in the serum exosomes of acute ischemic stroke patients. PLoS ONE 11, e0163645 (2016).
11. Yoon, Y. J., Kim, O. Y. & Gho, Y. S. Extracellular vesicles as emerging intercellular communicasomes. BMB Rep. 47, 531 (2014).
12. Chen, C. C. et al. Elucidation of exosome migration across the blood-brain barrier model in vitro. Cell. Mol. Bioeng. 9, 509-529 (2016).
13. van Kralingen, J. C. et al. Altered extracellular vesicle microRNA expression in ischemic stroke and small vessel disease. Transl. Stroke Res. 10, 495-508 (2019).
14. Moore, D. F. et al. Using peripheral blood mononuclear cells to determine a gene expression profile of acute ischemic stroke. Circulation 111, 212-221 (2005).
15. Théry, C., Amigorena, S., Raposo, G. & Clayton, A. Isolation and characterization of exosomes from cell culture supernatants and biological fluids. Curr. Protoc. Cell Biol. 30, 3.22.21-23.22.29 (2006).
16. Van Deun, J. et al. EV-TRACK: transparent reporting and centralizing knowledge in extracellular vesicle research. Nat. Methods 14, 228 (2017).
17. Contreras-Naranjo, J. C., Wu, H.-J. & Ugaz, V. M. Microfluidics for exosome isolation and analysis: enabling liquid biopsy for personalized medicine. Lab Chip 17, 3558-3577 (2017).
18. Reategui, E. et al. Engineered nanointerfaces for microfluidic isolation and molecular profiling of tumor-specific extracellular vesicles. Nat. Commun. 9, 175 (2018).
19. Fang, S. et al. Clinical application of a microfluidic chip for immunocapture and quantification of circulating exosomes to assist breast cancer diagnosis and molecular classification. PLoS ONE 12, e0175050 (2017).
20. Zhang, P. et al. Ultrasensitive detection of circulating exosomes with a 3D-nanopatterned microfluidic chip. Nat. Biomed. Eng. 3, 438-451 (2019).
21. Witek, M. A., Llopis, S. D., Wheatley, A., McCarley, R. L. & Soper, S. A. Purification and preconcentration of genomic DNA from whole cell lysates using photoactivated polycarbonate (PPC) microfluidic chips. Nucleic Acids Res. 34, e74 (2006).
22. Jackson, J. M. et al. UV activation of polymeric high aspect ratio microstructures: ramifications in antibody surface loading for circulating tumor cell selection. Lab Chip 14, 106-117 (2014).
23. Chang, K.-C. & Hammer, D. A. The forward rate of binding of surface-tethered reactants: effect of relative motion between two surfaces. Biophys. J. 76, 1280-1292 (1999).
24. Nair, S. V. et al. Enzymatic cleavage of uracil-containing single-stranded DNA linkers for the efficient release of affinity-selected circulating tumor cells. Chem. Commun. 51, 3266-3269 (2015).
25. Greenberg, J. M. et al. Immunophenotypic and cytogenetic analysis of Molt-3 and Molt-4: human T-lymphoid cell lines with rearrangement of chromosome 7. Blood 72, 1755-1760 (1988).
26. Selvaraj, U. M. & Stowe, A. M. Long-term T cell responses in the brain after an ischemic stroke. Discov. Med. 24, 323-333 (2017).
27. Tough, D. F., Sun, S. & Sprent, J. T cell stimulation in vivo by lipopolysaccharide (LPS). J. Exp. Med. 185, 2089-2094 (1997).
28. Hsu, H.-Y. & Wen, M.-H. Lipopolysaccharide-mediated reactive oxygen species and signal transduction in the regulation of interleukin-1 gene expression. J. Biol. Chem. 277, 22131-22139 (2002).
29. Rider, M. A., Hurwitz, S. N. & Meckes, D. G. Jr. ExtraPEG: a polyethylene glycol-based method for enrichment of extracellular vesicles. Sci. Rep. 6, 23978 (2016).
30. Yang, J., Li, C., Zhang, L. & Wang, X. Extracellular vesicles as carriers of non-coding RNAs in liver diseases. Front. Pharmacol. 9, 415 (2018).
31. Wei, Z. et al. Coding and noncoding landscape of extracellular RNA released by human glioma stem cells. Nat. Commun. 8, 1145 (2017).
32. Geis-Asteggiante, L. et al. Differential content of proteins, mRNAs, and miRNAs suggests that MDSC and their exosomes may mediate distinct immune suppressive functions. J. Proteome Res. 17, 486-498 (2017).
33. Tang, Y. et al. Gene expression in blood changes rapidly in neutrophils and monocytes after ischemic stroke in humans: a microarray study. J. Cereb. Blood Flow Metab. 26, 1089-1102 (2006).
34. Huang, X. et al. Characterization of human plasma-derived exosomal RNAs by deep sequencing. BMC Genomics 14, 319 (2013).
35. Chalela, J. A. et al. Magnetic resonance imaging and computed tomography in emergency assessment of patients with suspected acute stroke: a prospective comparison. Lancet 369, 293-298 (2007).
36. Huang, T. & Deng, C.-X. Current progresses of exosomes as cancer diagnostic and prognostic biomarkers. Int. J. Biol. Sci. 15, 1 (2019).
37. Labat-Gest, V. & Tomasi, S. Photothrombotic ischemia: a minimally invasive and reproducible photochemical cortical lesion model for mouse stroke studies. J. Vis. Exp. e50370 (2013).
38. Gale, B. et al. A review of current methods in microfluidic device fabrication and future commercialization prospects. Inventions 3, 60 (2018).
39. Pahattuge, T. et al. Visible-photorelease of liquid biopsy markers following microfluidic affinity-enrichment. Chem. Commun. 56, 4098-4101 (2020).
40. Soper, S. A. et al. Polymeric microelectromechanical systems. Anal. Chem. 72, 42A-651A (2000).
41. Wijerathne, H. et al. Affinity enrichment of extracellular vesicles from plasma reveals mRNA changes associated with a cute ischemic stroke. Commun. Biol. 3, 613 (2020).
42. Battle, K. N. et al. Solid-phase extraction and purification of membrane proteins using a UV-modified PMMA microfluidic bioaffinity microSPE device. The Analyst 139, 1355-1363, doi:10.1039/c3an02400h (2014).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1.-28. (canceled)

29. A method for identifying patients having an increased risk for acute ischemic stroke, comprising:
a) obtaining a biological sample of Extracellular Vesicles (EVs) from said patient
b) determining the expression levels of a cluster of at least three genes from Table 4 and Table 11, wherein upregulation of said markers relative to predetermined control levels observed in non-afflicted controls, are indicative of an increased risk for the development of acute ischemic stroke.

30. The method of claim 29 wherein said genes are PLBD1, FOS, MMP9, CA 4, and VCAN.

31. The method of claim 30, further comprising determining the expression levels of at least one other gene from Table 2.

32. The method of claim 29, wherein the predetermined levels are mean expression levels across the patient cohort.

33. The method of claim 29, wherein said determining step comprises contacting said sample with an agent having affinity for said ischemic stroke-associated markers, said agent forming a specific binding pair with said markers and further comprising a detectable label, measuring said detectable label, thereby determining expression level of said marker in said sample.

34. The method of claim 29, wherein said expression levels are determined using an input quality method.

35. The method of claim 29, wherein said marker comprise nucleic acids or fragments thereof, said agent is complementary nucleic acids which hybridizes to said marker and said marker is detected by in situ hybridization assay, hybridization assay, gel electrophoresis, RT-PCR, real time PCR, and microarray analysis.

36. The method of claim 29, further comprising administering an agent useful for the amelioration of stroke symptoms to said patient.

37. The method of claim 36, wherein said agent is recombinant tissue plasminogen activator (rt-PA), Tenecteplase, or mechanical thrombectomy.

38. The method of claim 36, wherein said agent is administered within about 4.5 hours after the onset of stroke symptoms.

39. The method of claim 36, wherein said agent is administered within about 9 hours after the onset of stroke symptoms.

40. The method of claim 29, wherein detection of the cluster comprises reverse transcribing RNA transcripts extracted from said sample into cDNA and amplifying said cDNA using primer pairs and performance of quantitative polymerase reaction (qPCR) thereby detecting and quantifying nucleic acids encoding said genes in said sample.

41. The method of claim 29, further comprising isolating said EVs from a biological sample from said patient.

42. The method of claim 41, wherein said EVs are isolated using EV microfluidic affinity purification (EV-MAP).

43. The method of claim 29, wherein said method is completed in about 1-9 hours.

44. The method of claim 43, wherein said method is completed within about 4.5 hours.

45. The method of claim 43, wherein said method is completed within about 3.7 hours.

46. The method of claim 43, wherein said method is completed within about 2.75 hours.


SEQUENCE LISTING (90 sequences)

SEQ ID NO: 1 - DNA, 21 bp, Artificial Sequence
  primer
aaatcccatgagtttcacgcc

SEQ ID NO: 2 - DNA, 24 bp, Artificial Sequence
  primer
ctcggctttgacagagtgcaagac

SEQ ID NO: 3 - DNA, 22 bp, Artificial Sequence
  primer
gccacaacaggtcgctcatccc

SEQ ID NO: 4 - DNA, 21 bp, Artificial Sequence
  primer
gctgctcggcgttctctcagg

SEQ ID NO: 5 - DNA, 24 bp, Artificial Sequence
  primer
gcatgcggttgctcaggatggaaa

SEQ ID NO: 6 - DNA, 25 bp, Artificial Sequence
  primer
aaacgacctgatcgctgcaaaatga

SEQ ID NO: 7 - DNA, 23 bp, Artificial Sequence
  primer
ccccttggtgccaacagatgagg

SEQ ID NO: 8 - DNA, 23 bp, Artificial Sequence
  primer
ggtgccaacagatgaggttcaca

SEQ ID NO: 9 - DNA, 22 bp, Artificial Sequence
  primer
cttagcagggtctgcgcttcgc

SEQ ID NO: 10 - DNA, 21 bp, Artificial Sequence
  primer
tacgatcagggtggcccggtg

SEQ ID NO: 11 - DNA, 22 bp, Artificial Sequence
  primer
tccagtgcgtgctgctcaacaa

SEQ ID NO: 12 - DNA, 21 bp, Artificial Sequence
  primer
gggcacctcttccattggggc

SEQ ID NO: 13 - DNA, 25 bp, Artificial Sequence
  primer
tcagcaaatgcaaagaagggagacc

SEQ ID NO: 14 - DNA, 20 bp, Artificial Sequence
  primer
actggccccgggaatcagct

SEQ ID NO: 15 - DNA, 21 bp, Artificial Sequence
  primer
ggccggctacatcgaggctct

SEQ ID NO: 16 - DNA, 20 bp, Artificial Sequence
  primer
cccctccgctggggcttact

SEQ ID NO: 17 - DNA, 23 bp, Artificial Sequence
  primer
gcgcctacggaaactcagccacc

SEQ ID NO: 18 - DNA, 22 bp, Artificial Sequence
  primer
tgagtcccgagcagcggaacaa

SEQ ID NO: 19 - DNA, 20 bp, Artificial Sequence
  primer
tggcaggctgggtccctctc

SEQ ID NO: 20 - DNA, 24 bp, Artificial Sequence
  primer
aaactctccatcacccagggtcag

SEQ ID NO: 21 - DNA, 23 bp, Artificial Sequence
  primer
gcaagtggacaccacaaaggcag

SEQ ID NO: 22 - DNA, 21 bp, Artificial Sequence
  primer
ctggccggcttcacaggaagt

SEQ ID NO: 23 - DNA, 21 bp, Artificial Sequence
  primer
ccgcgctcggggctctaagag

SEQ ID NO: 24 - DNA, 24 bp, Artificial Sequence
  primer
gggttacctcactgccccacacat

SEQ ID NO: 25 - DNA, 25 bp, Artificial Sequence
  primer
cgagcagaaggtgcagtctttgcaa

SEQ ID NO: 26 - DNA, 20 bp, Artificial Sequence
  primer
gggccgctcctactctgcct

SEQ ID NO: 27 - DNA, 21 bp, Artificial Sequence
  primer
cgatattcgggcagcgagggc

SEQ ID NO: 28 - DNA, 20 bp, Artificial Sequence
  primer
cctgaacctggccgtggctg

SEQ ID NO: 29 - DNA, 25 bp, Artificial Sequence
  primer
ttgaggggttaacattaggctggga

SEQ ID NO: 30 - DNA, 23 bp, Artificial Sequence
  primer
gcccctgaaccccagaacaacca

SEQ ID NO: 31 - DNA, 23 bp, Artificial Sequence
  primer
ggcggagaccacagtttggcaat

SEQ ID NO: 32 - DNA, 21 bp, Artificial Sequence
  primer
tcggccagtgcagagtcacac

SEQ ID NO: 33 - DNA, 24 bp, Artificial Sequence
  primer
gagctctgaagggagagactgtga

SEQ ID NO: 34 - DNA, 20 bp, Artificial Sequence
  primer
ggaccgccccttgcaggttt

SEQ ID NO: 35 - DNA, 20 bp, Artificial Sequence
  primer
ttcccgatcgccaggcagga

SEQ ID NO: 36 - DNA, 20 bp, Artificial Sequence
  primer
gaagtgcacgtcctgcggct

SEQ ID NO: 37 - DNA, 22 bp, Artificial Sequence
  primer
ctacgacaagtgccccaagctt

SEQ ID NO: 38 - DNA, 21 bp, Artificial Sequence
  primer
tccccagacaggggtagtgcg

SEQ ID NO: 39 - DNA, 20 bp, Artificial Sequence
  primer
cgcgcctccaaggtttcacg

SEQ ID NO: 40 - DNA, 23 bp, Artificial Sequence
  primer
ccagatacgtgggaggccaagag

SEQ ID NO: 41 - DNA, 22 bp, Artificial Sequence
  primer
cagacccctaactggtgctgtt

SEQ ID NO: 42 - DNA, 21 bp, Artificial Sequence
  primer
tccccgaggcctggcttatgg

SEQ ID NO: 43 - DNA, 24 bp, Artificial Sequence
  primer
ggctcagaatggcctccttttcca

SEQ ID NO: 44 - DNA, 24 bp, Artificial Sequence
  primer
tgtccagtgcttcaacccacaact

SEQ ID NO: 45 - DNA, 21 bp, Artificial Sequence
  primer
ggctcccccaaggtccaaagc

SEQ ID NO: 46 - DNA, 21 bp, Artificial Sequence
  primer
ggccgcaagcgactgttcctt

SEQ ID NO: 47 - DNA, 24 bp, Artificial Sequence
  primer
cggctgcctcttatatcccagaga

SEQ ID NO: 48 - DNA, 23 bp, Artificial Sequence
  primer
agccagcccccttcctttcctta

SEQ ID NO: 49 - DNA, 20 bp, Artificial Sequence
  primer
cgagcggtgtcagcgcctag

SEQ ID NO: 50 - DNA, 20 bp, Artificial Sequence
  primer
aggtgcctcggtcgagcaca

SEQ ID NO: 51 - DNA, 20 bp, Artificial Sequence
  primer
tctgcggtctgcccacgtac

SEQ ID NO: 52 - DNA, 21 bp, Artificial Sequence
  primer
gcagagccgcagccatctgaa

SEQ ID NO: 53 - DNA, 23 bp, Artificial Sequence
  primer
gaggatgacaggaatgcagggcc

SEQ ID NO: 54 - DNA, 22 bp, Artificial Sequence
  primer
tcaccctgtgccatttctggca

SEQ ID NO: 55 - DNA, 20 bp, Artificial Sequence
  primer
ccgtgtgccagtgctggtga

SEQ ID NO: 56 - DNA, 22 bp, Artificial Sequence
  primer
ggtctgtctccgcttggagtgt

SEQ ID NO: 57 - DNA, 22 bp, Artificial Sequence
  primer
tggtgctacactgggaccccac

SEQ ID NO: 58 - DNA, 20 bp, Artificial Sequence
  primer
gctgccttccccgcaggatt

SEQ ID NO: 59 - DNA, 20 bp, Artificial Sequence
  primer
tccccatggccctggcttgt

SEQ ID NO: 60 - DNA, 22 bp, Artificial Sequence
  primer
agctggagagggcaagggaagc

SEQ ID NO: 61 - DNA, 22 bp, Artificial Sequence
  primer
accaggcctctgcaagagcaac

SEQ ID NO: 62 - DNA, 20 bp, Artificial Sequence
  primer
tggtcccggcgacctcagtc

SEQ ID NO: 63 - DNA, 25 bp, Artificial Sequence
  primer
ggtggtttcttgcttgggtctaggg

SEQ ID NO: 64 - DNA, 22 bp, Artificial Sequence
  primer
gcggagcaatgtcccatgtccc

SEQ ID NO: 65 - DNA, 20 bp, Artificial Sequence
  primer
agccgctcctccaccgtgtt

SEQ ID NO: 66 - DNA, 20 bp, Artificial Sequence
  primer
accgtcgagtcagctcgggt

SEQ ID NO: 67 - DNA, 24 bp, Artificial Sequence
  primer
cttttccactctgcaggaagcctg

SEQ ID NO: 68 - DNA, 20 bp, Artificial Sequence
  primer
cggtgcggtggttctgggtc

SEQ ID NO: 69 - DNA, 20 bp, Artificial Sequence
  primer
gcagccttcagcgcaatggc

SEQ ID NO: 70 - DNA, 23 bp, Artificial Sequence
  primer
accatgtttcccagtctccgcgc

SEQ ID NO: 71 - DNA, 21 bp, Artificial Sequence
  primer
gacctcccacgactggtgtgc

SEQ ID NO: 72 - DNA, 20 bp, Artificial Sequence
  primer
gggggactggcggtccttct

SEQ ID NO: 73 - DNA, 24 bp, Artificial Sequence
  primer
aagagcatttcttctgggctccca

SEQ ID NO: 74 - DNA, 20 bp, Artificial Sequence
  primer
caccagggcgaggcacacag

SEQ ID NO: 75 - DNA, 20 bp, Artificial Sequence
  primer
ttgcctttgcgcagcaagcg

SEQ ID NO: 76 - DNA, 24 bp, Artificial Sequence
  primer
gcaacgatagggtttctcaccaca

SEQ ID NO: 77 - DNA, 21 bp, Artificial Sequence
  primer
gctggatggccacctgatccg

SEQ ID NO: 78 - DNA, 25 bp, Artificial Sequence
  primer
cattggtttccccaggtccatgacg

SEQ ID NO: 79 - DNA, 23 bp, Artificial Sequence
  primer
tccaggacagagccatagtgcgg

SEQ ID NO: 80 - DNA, 24 bp, Artificial Sequence
  primer
gcccccagttcttctcatcgttca

SEQ ID NO: 81 - DNA, 17 bp, Artificial Sequence
  primer
tgccaggaacacagtag

SEQ ID NO: 82 - DNA, 21 bp, Artificial Sequence
  primer
tctcaaagaaacagagtgata

SEQ ID NO: 83 - DNA, 22 bp, Artificial Sequence
  primer
gtactgagatgctaggtagata

SEQ ID NO: 84 - DNA, 18 bp, Artificial Sequence
  primer
gggatttacatggcactg

SEQ ID NO: 85 - DNA, 17 bp, Artificial Sequence
  primer
gaagcctggaacttgga

SEQ ID NO: 86 - DNA, 19 bp, Artificial Sequence
  primer
ttcagagagctggtagtta

SEQ ID NO: 87 - DNA, 17 bp, Artificial Sequence
  primer
agagccacagagcattt

SEQ ID NO: 88 - DNA, 20 bp, Artificial Sequence
  primer
caagggaaagtgactgatac

SEQ ID NO: 89 - DNA, 18 bp, Artificial Sequence
  primer
accgagagaaagcctatt

SEQ ID NO: 90 - DNA, 16 bp, Artificial Sequence
  primer
agcgcacggtgataaa